SEQUENCE LISTING (6 sequences)

SEQ ID NO: 1 - skipped

SEQ ID NO: 2 - DNA, 15 bp, synthetic construct
  source 1..15: mol_type = other DNA
cctattacgataaca

SEQ ID NO: 3 - DNA, 11 bp, synthetic construct
  source 1..11: mol_type = other DNA
  modified_base 7: mod_base = OTHER; note = locked nucleic acid
accacggtcac

SEQ ID NO: 4-5 - skipped (2 entries)

SEQ ID NO: 6 - DNA, 11 bp, synthetic construct
  source 1..11: mol_type = other DNA
  modified_base 4: mod_base = OTHER; note = locked nucleic acid
cgtctcctcag

(19) United States
(12) Patent Application Publication
(10) Pub. No.: US 2023/0340592 A1
(43) Pub. Date: Oct. 26, 2023

(54) TARGETED SEQUENCING

(71) Applicant: Singular Genomics Systems, Inc., San Diego, CA (US)

(72) Inventor: Eli N. GLEZER, Del Mar (CA)

(21) Appl. No.: 18/303,468

(22) Filed: Apr. 19, 2023

Related U.S. Application Data
(60) Provisional application No. 63/334,068, filed on Apr. 22, 2022.

(51) Int. Cl. C12Q 1/6874

(52) U.S. Cl. CPC C12Q 1/6874

(57) ABSTRACT

Disclosed herein, inter alia, are methods of making, amplifying, and sequencing compositions, and kits useful in obtaining sequencing data.

### CROSS-REFERENCES TO RELATED APPLICATIONS

This application claims the benefit of U.S. Provisional Application No. 63/334,068, filed Apr. 22, 2022, which is incorporated herein by reference in its entirety and for all purposes.

### SEQUENCE LISTING

The Sequence Listing written in file 051385-573001US_SEQUENCE_LISTING_ST26.xml, created Apr. 4, 2023, 1,839,712 bytes, machine format IBM-PC, MS Windows operating system, is hereby incorporated by reference.

### BACKGROUND

A number of next-generation sequencing (NGS) platforms are available for the high-throughput, massively parallel sequencing of nucleic acids. Certain NGS sequencing methodologies make use of simultaneously sequencing millions of fragments of nucleic acids, resulting in a 50,000-fold drop in the costs associated with sequencing since its inception. Due to the read lengths of current NGS platforms, ranging in length from about 35 to about 150 base pairs, nucleic acid sequencing technologies may struggle with accurately mapping sequences having large structural variations, e.g., inversions and translocations, tandem repeat regions, distinguishing clinically relevant genes from pseudogenes, and haplotype reconstructions. Disclosed herein, inter alia, are solutions to these and other problems in the art.

### BRIEF SUMMARY

In an aspect is provided a method of sequencing two or more regions (e.g., three regions) of a template nucleic acid, the method including: (a) contacting a first primer annealed to a first region of the template nucleic acid with a sequencing solution including a plurality of nucleotides and incorporating with a polymerase one or more nucleotides into the first primer to create a first extension strand, and detecting the one or more incorporated nucleotides so as to identify each incorporated nucleotide in the first extension strand; (b) contacting the template nucleic acid with a blocking element thereby terminating extension of the first extension strand thereby forming a blocked first extension strand; (c) contacting a second primer annealed to a second region of the template nucleic acid with a sequencing solution including a plurality of nucleotides and incorporating with a polymerase one or more nucleotides into the second primer to create a second extension strand, and detecting the one or more incorporated nucleotides so as to identify each incorporated nucleotide in the second extension strand; (d) contacting the template nucleic acid with a blocking element thereby terminating extension of the second extension strand and creating a blocked second extension strand; and (e) contacting a third primer annealed to a third region of the template nucleic acid with a sequencing solution including a plurality of nucleotides and incorporating with a polymerase one or more nucleotides into the third primer to create a third extension strand, and detecting the one or more incorporated nucleotides so as to identify each incorporated nucleotide in the third extension strand.

In an aspect is provided a method of forming an integrated strand complement of a template nucleic acid including a plurality of oligonucleotide probes, the method including: a. hybridizing one or more interposing oligonucleotide probes to the template nucleic acid, hybridizing a 5′ terminal oligonucleotide probe downstream of the one or more interposing oligonucleotide probes, and hybridizing a 3′ terminal oligonucleotide probe upstream of the one or more interposing oligonucleotide probes, wherein each of the interposing oligonucleotide probes includes from 5′ to 3′: i. a first hybridization sequence complementary to a first sequence of the template nucleic acid; ii. a loop region including a primer binding sequence; and iii. a second hybridization sequence complementary to a second sequence of the template nucleic acid; wherein the 5′ terminal oligonucleotide probe includes from 5′ to 3′: i. a hybridization sequence complementary to a third sequence of the template nucleic acid; and ii. a primer binding sequence; and wherein the 3′ terminal oligonucleotide probe includes from 3′ to 5′: i. a hybridization sequence complementary to a fourth sequence the template nucleic acid; and ii. a primer binding sequence; b. extending the 3′ end of each second hybridization sequence of the interposing oligonucleotide probes and the 3′ end of the hybridization sequence of the 3′ terminal oligonucleotide probe with one or more polymerases thereby forming an extension product of each of the oligonucleotide probes; c. ligating the 3′ end of each of the extension products to the 5′ end of the adjacent extension products, and ligating the 5′ end of the 5′ terminal oligonucleotide probe to the 3′ end of the adjacent extension product, each hybridized to the same template nucleic acid thereby making an integrated strand including a complement of the template nucleic acid including a plurality of the oligonucleotide probes; and d. amplifying the integrated strand by an amplification reaction to produce a complement of the integrated strand thereby forming an integrated strand complement of the template nucleic acid including oligonucleotide probes, wherein the complement of the integrated strand includes a complement of the plurality of oligonucleotide probes.

### BRIEF DESCRIPTION OF THE DRAWINGS

FIGS. 1A-1D show a schematic of the adapter sequences used in some embodiments. FIG. 1A shows examples of the adapter sequences, referred to as P1 and P2 adapters, respectively. The P1 adapter contains a first platform primer 1 (pp1) sequence, which is a sequence complementary to a first surface-immobilized primer, an optional index sequence (index), and a sequence complementary to a first sequencing primer (SP1). The P2 adapter contains a second platform primer 2 (pp2) sequence, which is a sequence complementary to a second surface-immobilized primer, an optional index sequence (index), and a region complementary to a second sequencing primer (SP2). The dashed lines are indicative of regions within the adapter and are included merely to aid the eye in the different arrangement of the sequences and are not indicative of the overall size/length (i.e., the index sequence may not be the same length as the sequencing primer despite the illustration showing the index sequence and sequencing primer as being the same size). FIG. 1B shows various adapter configurations used in some embodiments. The top panel illustrates an embodiment of a Y adapter including (i) a first strand having a 5′-arm and a 3′-portion, and (ii) a second strand having a 5′-portion and a 3′-arm, wherein the 3′-portion of the first strand is substantially complementary to the 5′-portion of the second strand, and the 5′-arm of the first strand is not substantially complementary to the 3′-arm of the second strand. In this embodiment, the complementary portions (i.e., duplex regions) of the Y adapter include a melting temperature (Tm) of about 40-45° C. and a length of about 10 to 15 nucleotides. In embodiments, the complementary portions (i.e., duplex regions) of the Y adapter include a Tm (melting temperature) of about 35-45° C. or 30-45° C. and a length of about 12 bases. The middle panel shows an embodiment of a hairpin adapter including a 5′-end, a 5′ portion, a loop, a 3′ portion and a 3′-end. In this embodiment, a duplex region of the hairpin adapter includes a Tm (melting temperature) of about 40-45° C. and a length of about 10-16 bases. In embodiments, the duplex region of the adapter includes a Tm (melting temperature) of about 35-45° C. or 30-45° C. and a length of about 12 bases. The bottom panel illustrates an embodiment of a hairpin adapter, which includes a double stranded (stem) region and a loop region. Within the loop region is a priming site (P3) and optionally an index, alternatively referred to as a unique molecular identifier (UMI). FIG. 1C illustrates the adapters may include different duplex ends. For example, the double-stranded region of a Y adapter (alternatively referred to as a forked adapter) may be blunt-ended (top), have a 3′ overhang (middle), or a 5′ overhang (bottom). On the right are embodiments of hairpin adapters, each including a 5′-end and a 3′-end. In some embodiments, a hairpin adapter includes a double stranded portion (a double-stranded “stem” region) and a loop, where 5′P refers to a phosphorylated 5′ end. A double-stranded stem region of a hairpin adapter may be blunt-ended (top), it may have a 5′ overhang (middle), or a 3′ overhang (bottom). An overhang may include a single nucleotide or more than one nucleotide. FIG. 1D illustrates a double-stranded template nucleic acid prepared according to standard library prep methods (e.g., fragmenting, polishing, A-tailing, etc.). Adapters P1 and P2′, or alternatively P1′ and P2 are ligated to each of the ends of the template. For clarity, the ssDNA is depicted showing an embodiment of the P1 and P2′ adapters, wherein each adapter includes a platform priming sequence, referred to as pp1 or pp2′, and a sequencing primer sequence, referred to as SP1 or SP2′, for P1 and P2′, respectively.

FIG. 2 illustrates an embodiment of sequencing a plurality of regions on the same single polynucleotide strand. For example, to genomic DNA with known regions, two or more different primers are annealed to the known regions and sequenced in an iterative manner. The first sequencing primer (e.g., SP1) is hybridized to an immobilized template polynucleotide including primer binding sequences on each end (e.g., a P2 primer binding sequence on the 5′ end and a P1′ primer binding sequence on the 3′ end), wherein the template polynucleotide is immobilized to a solid support (illustrated as a dark rectangle on the right), followed by sequencing a first region by extending the primer with a polymerase to incorporate and detect labeled nucleotides (depicted as a dashed line and star). Following sequencing of the first region, extension is terminated (e.g., by incorporating a blocking element, such as a dideoxynucleotide triphosphate (ddNTP) illustrated as an octagon), or by removing the sequenced strand). This process is repeated to obtain multiple sequencing reads on a single polynucleotide. Next, a second sequencing primer (e.g., SP2) is hybridized to a second region of the insert and the second region is then sequenced. Sequencing of the second region is terminated, and then a third sequencing primer (e.g., SP3) is hybridized to a third region of the insert and sequencing of the third region is performed. Once sequencing of the third region is complete, extension is terminated once again. Although only three regions of the template polynucleotide as shown as being targeted by sequencing primers and sequenced, it will be understood that additional primers may be designed to target additional regions for sequencing.

FIGS. 3A-3D illustrate interposing probes (IPP) as described herein. FIG. 3A is an overview of a non-limiting example of an interposing probe. In embodiments, the interposing probe consists of the hybridization sequences and loop (e.g., an IPP without the stem regions).

FIG. 3B shows an interposing probe subjected to denaturing conditions (i.e., the stem regions are no longer hybridized together). FIG. 3C shows a 3′ flanking adapter, alternatively referred to herein as a 3′ terminal oligonucleotide probe, including a P1 primer binding sequence on the 5′ end and a hybridization sequence on the 3′ end. FIG. 3D shows a 5′ flanking adapter, alternatively referred to herein as a 5′ terminal oligonucleotide probe, including a P2 primer binding sequence on the 3′ end and a hybridization sequence on the 5′ end.

FIG. 4 illustrates an amplification process integrating the interposing probes (IPPs) as described herein to form an integrated strand. Depicted in the top panel is a single-stranded template DNA molecule, to which a plurality of IPPs and two flanking adapters are hybridized (e.g., IPPs and flanking adapters as described in FIGS. 3A-3D). Next, a polymerase (depicted as a cloud-like object) extends from the 3′ end of each hybridized IPP and 3′ terminal adapter and halts extension at or around the next IPP or flanking adapter. A ligase (not shown) then ligates the extended strands, IPPs, and flanking adapters together to produce a long, continuous DNA strand which contains integrated probes and complements of the original template DNA molecule. When the hairpin stems are not hybridized together, the resultant single-stranded polynucleotide is shown in the bottom panel. Note, the shading/coloring used in the figures is not indicative of an identical sequence. For example, although the loops depicted in the top panel of FIG. 4 are rendered in the same color/shading, this does not imply the sequences of the loops are identical. In embodiments, the only sequences that are common are the stems of the interposing probes.

FIGS. 5A-5C illustrate an embodiment of a sequencing process of an interposing probe-containing single-stranded polynucleotide immobilized on a substrate. FIG. 5A shows hybridization of a first sequencing primer (e.g., SP1) to the flanking adapter on the 3′ end of the DNA strand and extension in the presence of a polymerase and detectable nucleotides (shown as a star) the first region of the polynucleotide. Next, sequencing is terminated through the incorporation of a blocking element, for example, a ddNTP (shown here as an octagon). A second sequencing primer (e.g., SP2) is then hybridized to a sequence of the first interposing probe (e.g., the loop portion), and a second region of the polynucleotide is sequenced in the presence of a polymerase and detectable nucleotides, followed by termination of extension through incorporation of another blocking element (e.g., a ddNTP). As shown in FIG. 5B, a third sequencing primer (e.g., SP3) is then hybridized to a sequence of the second interposing probe (e.g., the loop portion), and a third region of the polynucleotide is sequenced in the presence of a polymerase and detectable nucleotides. Sequencing of the third region is then terminated through incorporation of a third blocking element (e.g., a ddNTP). A fourth sequencing primer is then hybridized to a sequence of the third interposing probe (e.g., the loop portion), and a fourth region (e.g., the 5′ end) of the polynucleotide is sequenced in the presence of a polymerase and detectable nucleotides. In embodiments, the sequencing primer hybridizes to the loop region, the stem region, or the hybridization sequence region. In embodiments, the sequencing primer hybridizes to the loop region. In embodiments, the sequencing primer hybridizes to the stem region (e.g., one of the two complementary stem regions of the IPP). In embodiments, the sequencing primer hybridizes to the hybridization sequence (e.g., one of the two hybridization sequence regions of the IPP). Sequencing of the fourth region is then terminated through incorporation of a fourth blocking element (e.g., ddNTP). While each sequencing primer and corresponding sequenced region of the template polynucleotide are illustrated as being spaced in regular intervals, it is understood that each sequenced region may be of varying lengths, and sequencing primers may be targeted to non-adjacent portions of the template polynucleotide. FIG. 5C illustrates the process of bioinformatically reconstructing and aligning variable-length sequencing reads based on known features, e.g., sequencing primer binding sequences. In this example, reads with matching features are surrounded by a dashed box.

FIG. 6 illustrates an alternate embodiment for integrating a probe insert (i.e., a probe oligonucleotide complementary to a sequencing primer) into a target nucleic acid (e.g., DNA) strand utilizing a protein integration complex (PIC). The top panel illustrates the target DNA (e.g., a single-stranded polynucleotide), and the components of the PIC, including a probe insert, a Cas or Argonaute protein (e.g., a Cas protein (e.g., Cas9), a Argonaute (e.g., Natronobacterium gregorgi Argonaute, or NgAgo), or a variant thereof), and a guide nucleic acid (e.g., a guide RNA or guide 5′ phosphorylated single-stranded nucleic acid), wherein the guide nucleic acid includes a target-specific nucleotide region complementary or substantially complementary to a region of the target nucleic acid. The middle panel illustrates targeting of the probe insert by the Cas9 or NgAgo protein complex to the guide nucleic acid-specific target region. Target nucleic acid cleavage and probe insert integration then occur, for example, with additional factors such as a polymerase (not shown). The bottom panel illustrates the resulting template nucleic acid containing the integrated probe inserts, which may then be amplified and sequenced, for example, using a sequencing process as illustrated in FIGS. 5A-5B with sequencing primers complementary to the probe insert sequences.

### DETAILED DESCRIPTION

Described herein are compositions and methods for mapping long (e.g., 1 kB-10 kB) sequences, which are especially useful for sequences having large structural variations, e.g., inversions and translocations, tandem repeat regions, distinguishing clinically relevant genes from pseudogenes, and haplotype reconstructions.

### I. Definitions

All patents, patent applications, articles and publications mentioned herein, both supra and infra, are hereby expressly incorporated herein by reference in their entireties. The practice of the technology described herein will employ, unless indicated specifically to the contrary, conventional methods of chemistry, biochemistry, organic chemistry, molecular biology, bioinformatics, microbiology, recombinant DNA techniques, genetics, immunology, and cell biology that are within the skill of the art, many of which are described below for the purpose of illustration. Examples of such techniques are available in the literature. See, e.g., Singleton et al., DICTIONARY OF MICROBIOLOGY AND MOLECULAR BIOLOGY 2nd ed., J. Wiley & Sons (New York, NY 1994); and Sambrook and Green, Molecular Cloning: A Laboratory Manual, 4th Edition (2012). Methods, devices and materials similar or equivalent to those described herein can be used in the practice of this invention.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

As used herein, the singular terms “a”, “an”, and “the” include the plural reference unless the context clearly indicates otherwise. Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present.

As used herein, the term “control” or “control experiment” is used in accordance with its plain and ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

As used herein, the term “associated” or “associated with” can mean that two or more species are identifiable as being co-located at a point in time. An association can mean that two or more species are or were within a similar container. An association can be an informatics association, where for example digital information regarding two or more species is stored and can be used to determine that one or more of the species were co-located at a point in time. An association can also be a physical association.

As used herein, the term “complementary” or “substantially complementary” refers to the hybridization, base pairing, or the formation of a duplex between nucleotides or nucleic acids. For example, complementarity exists between the two strands of a double-stranded DNA molecule or between an oligonucleotide primer and a primer binding site on a single-stranded nucleic acid when a nucleotide (e.g., RNA or DNA) or a sequence of nucleotides is capable of base pairing with a respective cognate nucleotide or cognate sequence of nucleotides. When referring to a double-stranded polynucleotide including a first strand hybridized to a second strand, it is to be understood that each of the terms “first strand” and “second strand” refer to single-stranded polynucleotides. As described herein and commonly known in the art the complementary (matching) nucleotide of adenosine (A) is thymidine (T) and the complementary (matching) nucleotide of guanosine (G) is cytosine (C). Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. “Duplex” means at least two oligonucleotides and/or polynucleotides that are fully or partially complementary undergo Watson-Crick type base pairing among all or most of their nucleotides so that a stable complex is formed. Complementary single stranded nucleic acids and/or substantially complementary single stranded nucleic acids can hybridize to each other under hybridization conditions, thereby forming a nucleic acid that is partially or fully double stranded. When referring to a double-stranded polynucleotide including a first strand hybridized to a second strand, it is understood that each of the first strand and the second strand are independently single-stranded polynucleotides. All or a portion of a nucleic acid sequence may be substantially complementary to another nucleic acid sequence, in some embodiments. As referred to herein, “substantially complementary” refers to nucleotide sequences that can hybridize with each other under suitable hybridization conditions. Hybridization conditions can be altered to tolerate varying amounts of sequence mismatch within complementary nucleic acids that are substantially complementary. Substantially complementary portions of nucleic acids that can hybridize to each other can be 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other. In some embodiments substantially complementary portions of nucleic acids that can hybridize to each other are 100% complementary. Nucleic acids, or portions thereof, that are configured to hybridize to each other often include nucleic acid sequences that are substantially complementary to each other.

As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that complement one another (e.g., about 60%, preferably 65%, 70%, 75%, 80%, 85%, 90%, 91%, 92%, 93%, 94%, 95%, 96%, 97%, 98%, 99%, or higher complementarity over a specified region). In embodiments, two sequences are complementary when they are completely complementary (e.g., having 100% complementarity). In embodiments, sequences in a pair of complementary sequences form portions of a single polynucleotide with non-base-pairing nucleotides (e.g., as in a hairpin or loop structure, with or without an overhang) or portions of separate polynucleotides. In embodiments, one or both sequences in a pair of complementary sequences form portions of longer polynucleotides, which may or may not include additional regions of complementarity.

As used herein, the term “contacting” is used in accordance with its plain ordinary meaning and refers to the process of allowing at least two distinct species (e.g., chemical compounds including biomolecules or cells) to become sufficiently proximal to react, interact or physically touch. However, the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be a compound, nucleic acid, a protein, or enzyme (e.g., a DNA polymerase).

As used herein, the terms “nucleic acid,” “nucleic acid molecule,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may include natural nucleic acid sequences and variants thereof, artificial nucleic acid sequences, or a combination of such sequences. As may be used herein, the terms “nucleic acid oligomer” and “oligonucleotide” are used interchangeably and are intended to include, but are not limited to, nucleic acids having a length of 200 nucleotides or less. In some embodiments, an oligonucleotide is a nucleic acid having a length of 2 to 200 nucleotides, 2 to 150 nucleotides, 5 to 150 nucleotides or 5 to 100 nucleotides. The terms “polynucleotide,” “oligonucleotide,” “oligo” or the like refer, in the usual and customary sense, to a linear sequence of nucleotides. Oligonucleotides are typically from about 5, 6, 7, 8, 9, 10, 12, 15, 25, 30, 40, 50 or more nucleotides in length, up to about 100 nucleotides in length. In some embodiments, an oligonucleotide is a primer configured for extension by a polymerase when the primer is annealed completely or partially to a complementary nucleic acid template. A primer is often a single stranded nucleic acid. In certain embodiments, a primer, or portion thereof, is substantially complementary to a portion of an adapter. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. In some embodiments, an oligonucleotide may be immobilized to a solid support.

As used herein, the terms “polynucleotide primer” and “primer” refers to any polynucleotide molecule that may hybridize to a polynucleotide template, be bound by a polymerase, and be extended in a template-directed process for nucleic acid synthesis. The primer may be a separate polynucleotide from the polynucleotide template, or both may be portions of the same polynucleotide (e.g., as in a hairpin structure having a 3′ end that is extended along another portion of the polynucleotide to extend a double-stranded portion of the hairpin). Primers (e.g., forward or reverse primers) may be attached to a solid support (e.g., a polymer coated solid support). In embodiments, forward primers anneal to the antisense strand of the double-stranded DNA, which runs from the 3′ to 5′ direction. Forward primers, for example, initiate the synthesis of a gene in the 5′ to 3′ direction. In embodiments, reverse primers anneal to the sense strand of the double-stranded DNA, which runs from the 5′ to 3′ direction. Reverse primers, for example, initiate the synthesis of a gene in the 3′ to 5′ direction. A primer can be of any length depending on the particular technique it will be used for. For example, PCR primers are generally between 10 and 40 nucleotides in length. The length and complexity of the nucleic acid fixed onto the nucleic acid template may vary. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. One of skill can adjust these factors to provide optimum hybridization and signal production for a given hybridization procedure. The primer permits the addition of a nucleotide residue thereto, or oligonucleotide or polynucleotide synthesis therefrom, under suitable conditions. In an embodiment the primer is a DNA primer, i.e., a primer consisting of, or largely consisting of, deoxyribonucleotide residues. The primers are designed to have a sequence that is the complement of a region of template/target DNA to which the primer hybridizes. The addition of a nucleotide residue to the 3′ end of a primer by formation of a phosphodiester bond results in a DNA extension product. The addition of a nucleotide residue to the 3′ end of the DNA extension product by formation of a phosphodiester bond results in a further DNA extension product. In another embodiment the primer is an RNA primer. In embodiments, a primer is hybridized to a target polynucleotide. A “primer” is complementary to a polynucleotide template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

A “blocking element” refers to an agent (e.g., polynucleotide, protein, nucleotide) that reduces and/or inhibits nucleotide incorporation (i.e., extension of a primer) relative to the absence of the blocking element. In embodiments, the blocking element is a nucleotide containing a chain-terminating nucleotide, where the chain-terminating nucleotide is a 3′ dideoxynucleotide, reversibly-terminated deoxynucleotide, or a modified nucleotide triphosphate that lacks a 3′-OH. In embodiments, the blocking element including incorporating a ddNTP into a 3′ end of the extended strand. In embodiments, the blocking element is a non-extendable oligomer (e.g., a 3′-blocked oligo). A blocking element on a nucleotide can be reversible, whereby the blocking moiety can be removed or modified to allow the 3′ hydroxyl to form a covalent bond with the 5′ phosphate of another nucleotide. For example, a reversible terminator may refer to a blocking moiety located, for example, at the 3′ position of the nucleotide and may be a chemically cleavable moiety such as an allyl group, an azidomethyl group or a methoxymethyl group. In embodiments the blocking moiety is not reversible (e.g., the blocking element including a blocking moiety irreversibly prevents extension). In embodiments, the blocking element includes an oligo having a 3′ dideoxynucleotide or similar modification to prevent extension by a polymerase and is used in conjunction with a non-strand displacing polymerase. In another example implementation, the blocking element includes one or more modified nucleotides including a cleavable linker (e.g., linked to the 5′, 3′, or the nucleobase) containing PEG, thereby blocking the extension. In another example implementation, the blocking element includes one or more modified nucleotides linked to biotin, to which a protein (e.g., streptavidin) can be bound, thereby blocking polymerase extension. In another example implementation, the blocking element includes a modified nucleotide, such as iso dGTP or iso dCTP, which are complementary to each other. In a reaction of polymerization lacking the appropriate complementary modified nucleotides, the extension of a primer is halted. In another example implementation, the blocking element includes one or more sequences which is recognized and bound by one or more single-stranded DNA-binding proteins, thereby blocking polymerase extension at the bound site. In another example implementation, the blocking element includes one or more sequences which are recognized and bound by one or more short RNA or PNA oligos, thereby blocking the extension by a DNA polymerase that cannot strand displace RNA or PNA. In embodiments, the blocking element includes locked nucleic acids (LNAs), Bis-locked nucleic acids (bisLNAs), twisted intercalating nucleic acids (TINAs), bridged nucleic acids (BNAs), 2′-O-methyl RNA:DNA chimeric nucleic acids, minor groove binder (MGB) nucleic acids, morpholino nucleic acids, C5-modified pyrimidine nucleic acids, peptide nucleic acids (PNAs), phosphorothioate nucleic acids, or combinations thereof. In embodiments, the blocking element includes phosphorothioate nucleic acids. In embodiments, the blocking element includes one or more locked nucleic acids (LNAs), 2-amino-deoxyadenosine (2-amino-dA), trimethoxystilbene-functionalized oligonucleotides (TFOs), Pyrene-functionalized oligonucleotides (PFOs), peptide nucleic acids (PNAs), or aminoethyl-phenoxazine-dC (AP-dC) nucleic acids. In embodiments, the blocking element includes 10 to 15 locked nucleic acids (LNAs). In embodiments, the blocking element includes one or more phosphorothioates at the 5′ end. In embodiments, the blocking element includes one or more LNAs at the 5′ end. In embodiments, the blocking element includes two or more consecutive LNAs at the 3′ end. In embodiments, the blocking element includes two or more consecutive LNAs at the 5′ end. In embodiments, the blocking element includes a plurality (e.g., 2 to 10) of synthetic nucleotides (e.g., LNAs) and a plurality (e.g., 2 to 10) canonical or native nucleotides (e.g., dNTPs). In embodiments, the blocking element includes one or more (e.g., 2 to 5) deoxyuracil nucleobases (dU). In embodiments, the one or more dU nucleobases are at or near the 3′ end of the blocking element (e.g., within 5 nucleotides of the 3′ end). In embodiments, the one or more dU nucleobases are distributed through the blocking element. In embodiments, the blocking element includes from 5′ to 3′ a plurality (e.g., 2 to 5) of phosphorothioate nucleic acids, followed by a plurality of synthetic nucleotides (e.g., LNAs), and a plurality (e.g., 2 to 10) of canonical bases. In embodiments, the blocking element is about 10 to 100 nucleotides in length. In embodiments, the blocking element is about 15 to about 40 nucleotides in length. In embodiments, the calculated or predicted melting temperature (Tm) of the blocking element is about 70° C. to about 95° C. In embodiments, the calculated or predicted melting temperature (Tm) of the blocking element is about 75° C. to about 85° C. In embodiments, the calculated or predicted melting temperature (Tm) of the blocking element is 75° C. to 85° C.

As used herein, the terms “hybridization sequence” or “hybridization pad” refers to one or both of two regions (e.g., “first hybridization sequence” and “second hybridization sequence”) on either end of an interposing oligonucleotide probe that are capable of hybridizing to single-stranded template nucleic acids. In embodiments, hybridization sequences are a complement to the original target nucleic acid. In embodiments, each hybridization sequence is composed of about 3 to about 40 nucleotides. In embodiments, each hybridization sequence is composed of about 3 to about 5 nucleotides. In embodiments, the first hybridization sequence includes about 3 to about 5 nucleotides and the second hybridization sequence includes about 3 to 25 nucleotides. In embodiments, the first hybridization sequence includes about 5 to about 15 nucleotides and the second hybridization sequence includes about 5 to 15 nucleotides. In embodiments, the first hybridization sequence includes about 10 to about 15 nucleotides and the second hybridization sequence includes about 10 to 15 nucleotides. In embodiments, the hybridization sequence includes a targeted primer sequence, or a portion thereof. A “targeted primer sequence” refers to a nucleic acid sequence that is complementary to a known nucleic acid region (e.g., complementary to a universally conserved region, or complementary sequences to target specific genes or mutations that have relevancy to a particular cancer phenotype). The hybridization sequences may include sequences designed through computational software, e.g., Primer BLAST, LaserGene (DNAStar), Oligo (National Biosciences, Inc.), MacVector (Kodak/IBI) or the GCG suite of programs to optimize desired properties. In embodiments, the hybridization sequence includes a limited-diversity sequence. A “limited-diversity sequence” refers to a nucleic acid sequence that includes random nucleotide regions and fixed nucleotide regions (e.g., NNANN, ANNTN, TNCNA, etc., wherein N represents random nucleotides and A, T, C, G represent fixed nucleotides). In embodiments, each hybridization sequence is composed of 3 random nucleotides and 1 to 2 non-random nucleotides. In embodiments, each hybridization sequence is composed of 4 random nucleotides and 1 to 2 non-random nucleotides.

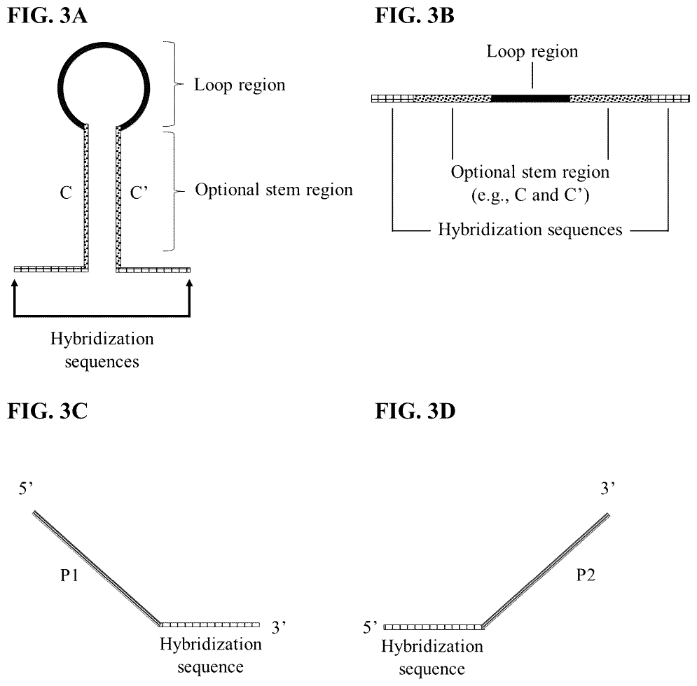

As used herein, the term “stem region” or “stem” refers to a region of an interposing oligonucleotide probe that includes two known sequences capable of hybridizing to each other. In embodiments, the stem includes about 5 to about 10 nucleotides, and is stable (i.e., capable to remaining hybridized together) at approximately 37° C., and unhybridizes (i.e., denatures) at temperatures greater than 50° C. As the stem is of known or pre-determined sequence (i.e., non-random sequence), the stem sequences allow for location identification of interposing oligonucleotide probes. In embodiments, the stem region includes two regions of the same strand that are complementary separated by a loop region; see for example FIG. 3A.

As used herein, the term “loop region” or “loop” refers to a region of an interposing oligonucleotide that is between sequences of the stem region, and remains single-stranded when sequences of the stem region are hybridized to one another. In embodiments, the loop includes about 10 to about 20 random nucleotides. In embodiments, the loop includes a modified nucleotide (e.g., a nucleotide linked to an affinity tag). In embodiments, the loop includes a biotinylated nucleotide (e.g., biotin-11-cytidine-5′-triphosphate). In embodiments, the loop region includes a barcode sequence. See, for example, FIG. 3A. In embodiments, the loop includes a limited-diversity sequence. For example, in embodiments, the loop includes a TT-[UMI]-TT sequence, such as TT-[NNNNNNNNNNNN]-TT (SEQ ID NO:1) sequence, wherein N represents random nucleotides and A, T, C, G represent fixed nucleotides). In embodiments, the loop includes a primer binding sequence. In embodiments, the loop includes a sequencing primer binding sequence.

As used herein, the terms “interposing oligonucleotide probes”, “IPP”, or “probe insert” refer to hairpin oligonucleotides with complementary sequences to target regions on a specific polynucleotide, where multiple oligonucleotide probes are capable of selectively hybridizing with different regions of a single polynucleotide and therefore the oligonucleotide probes are regarded to be “interposing”. Interposing oligonucleotide probes, as described herein, contain, from 5′ to 3′, a first hybridization sequence complementary to a first sequence of the template nucleic acid, a loop region including a primer binding sequence, and a second hybridization sequence complementary to a second sequence of the template nucleic acid.

As used herein, the terms “terminal oligonucleotide probes” or “flanking adapters” refer to oligonucleotides capable of selectively hybridizing with the sequence at the 5′ or 3′ terminus of a target nucleic acid. Terminal oligonucleotide probes are referred to as 5′ terminal oligonucleotide probes or 3′ terminal oligonucleotide probes. In embodiments, 5′ terminal oligonucleotide probes contain a hybridization sequence to the target nucleic acid at the 5′ end and primer binding sites (e.g., platform binding sequence and sequencing primer sequence) at the 3′ end, and 3′ terminal oligonucleotide probes contain a hybridization sequence to the target nucleic acid at the 3′ end and the primer binding sites (e.g., platform binding sequence and sequencing primer sequence) at the 5′ end. In embodiments, the 5′ and 3′ terminal oligonucleotide probes contain a first hybridization sequence to the template nucleic acid, a loop region containing a primer binding sequence, and a second hybridization sequence to a second region on the template nucleic acid.

As used herein, the term “integrated strand” refers to the long, continuous single-stranded DNA strand that contains the sequence of the 5′ terminal oligonucleotide probe, the sequence of the interposing oligonucleotide probes, the complement of the original template DNA molecule, and the sequence of the 3′ terminal oligonucleotide probe. The integrated stand as described herein is a result of hybridizing the terminal oligonucleotide probes and interposing oligonucleotide probes to the target template DNA molecule, extending the 3′ end of each hybridized terminal and interposing oligonucleotide probes by a polymerase, and ligation of adjacent sequences by a ligase. In embodiments, the integrated strand contains the complete sequences of the 5′ terminal oligonucleotide probes, the interposing oligonucleotide probes, the 3′ terminal oligonucleotide probes, and the complement sequence of a single template nucleic acid.

As used herein, the term “random” in the context of a nucleic acid sequence or barcode sequence refers to a sequence where one or more nucleotides has an equal probability of being present. In embodiments, one or more nucleotides is selected at random from a set of two or more different nucleotides at one or more positions, with each of the different nucleotides selected at one or more positions represented in a pool of oligonucleotides including the random sequence. For example, a random sequence may be represented by a sequence composed of N's, where N can be any nucleotide (e.g., A, T, C, or G). For example, a four base random sequence may have the sequence NNNN, where the Ns can independently be any nucleotide (e.g., AATC). Interposing oligonucleotide probes that contain a random sequence, collectively, have sequences composed of Ns within the hybridization sequences, stem region, or loop region.

As used herein, the terms “solid support” and “substrate” and “solid surface” are used interchangeably and refers to discrete solid or semi-solid surfaces to which a plurality of nucleic acid (e.g., primers) may be attached. A solid support may encompass any type of solid, porous, or hollow sphere, ball, cylinder, or other similar configuration composed of plastic, ceramic, metal, or polymeric material (e.g., hydrogel) onto which a nucleic acid may be immobilized (e.g., covalently or non-covalently). A solid support may include a discrete particle that may be spherical (e.g., microspheres) or have a non-spherical or irregular shape, such as cubic, cuboid, pyramidal, cylindrical, conical, oblong, or disc-shaped, and the like. Solid supports may be in the form of discrete particles, which alone does not imply or require any particular shape. The term “particle” means a small body made of a rigid or semi-rigid material. The body can have a shape characterized, for example, as a sphere, oval, microsphere, or other recognized particle shape whether having regular or irregular dimensions. As used herein, the term “discrete particles” refers to physically distinct particles having discernible boundaries. The term “particle” does not indicate any particular shape. The shapes and sizes of a collection of particles may be different or about the same (e.g., within a desired range of dimensions, or having a desired average or minimum dimension). A particle may be substantially spherical (e.g., microspheres) or have a non-spherical or irregular shape, such as cubic, cuboid, pyramidal, cylindrical, conical, oblong, or disc-shaped, and the like. In embodiments, the particle has the shape of a sphere, cylinder, spherocylinder, or ellipsoid. Discrete particles collected in a container and contacting one another will define a bulk volume containing the particles, and will typically leave some internal fraction of that bulk volume unoccupied by the particles, even when packed closely together. In embodiments, cores and/or core-shell particles are approximately spherical. As used herein the term “spherical” refers to structures which appear substantially or generally of spherical shape to the human eye, and does not require a sphere to a mathematical standard. In other words, “spherical” cores or particles are generally spheroidal in the sense of resembling or approximating to a sphere. In embodiments, the diameter of a spherical core or particle is substantially uniform, e.g., about the same at any point, but may contain imperfections, such as deviations of up to 1, 2, 3, 4, 5 or up to 10%. Because cores or particles may deviate from a perfect sphere, the term “diameter” refers to the longest dimension of a given core or particle. Likewise, polymer shells are not necessarily of perfect uniform thickness all around a given core. Thus, the term “thickness” in relation to a polymer structure (e.g., a shell polymer of a core-shell particle) refers to the average thickness of the polymer layer.

A solid support may further include a polymer or hydrogel on the surface to which the primers are attached (e.g., the primers are covalently attached to the polymer, wherein the polymer is in direct contact with the solid support). Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefin copolymers, polyimides etc.), nylon, ceramics, resins, Zeonor, silica or silica-based materials including silicon and modified silicon, carbon, metals, inorganic glasses, optical fiber bundles, photopatternable dry film resists, UV-cured adhesives and polymers. The solid supports for some embodiments have at least one surface located within a flow cell. The solid support, or regions thereof, can be substantially flat. The solid support can have surface features such as wells, pits, channels, ridges, raised regions, pegs, posts or the like. The term solid support is encompassing of a substrate (e.g., a flow cell) having a surface including a polymer coating covalently attached thereto. In embodiments, the solid support is a flow cell. The term “flow cell” as used herein refers to a chamber including a solid surface across which one or more fluid reagents can be flowed. Examples of flow cells and related fluidic systems and detection platforms that can be readily used in the methods of the present disclosure are described, for example, in Bentley et al., Nature 456:53-59 (2008). In certain embodiments a substrate includes a surface (e.g., a surface of a flow cell, a surface of a tube, a surface of a chip, surface of a particle), for example a metal surface (e.g., steel, gold, silver, aluminum, silicon and copper). In some embodiments a substrate (e.g., a substrate surface) is coated and/or includes functional groups and/or inert materials. In certain embodiments a substrate includes a bead, a chip, a capillary, a plate, a membrane, a wafer (e.g., silicon wafers), a comb, or a pin for example. In some embodiments a substrate includes a bead and/or a nanoparticle. A substrate can be made of a suitable material, non-limiting examples of which include a plastic or a suitable polymer (e.g., polycarbonate, poly(vinyl alcohol), poly(divinylbenzene), polystyrene, polyamide, polyester, polyvinylidene difluoride (PVDF), polyethylene, polyurethane, polypropylene, and the like), borosilicate, silica, nylon, Wang resin, Merrifield resin, metal (e.g., iron, a metal alloy, sepharose, agarose, polyacrylamide, dextran, cellulose and the like or combinations thereof. In some embodiments a substrate includes a magnetic material (e.g., iron, nickel, cobalt, platinum, aluminum, and the like). In certain embodiments a substrate includes a magnetic bead (e.g., DYNABEADS®, hematite, AMPure XP). Magnets can be used to purify and/or capture nucleic acids bound to certain substrates (e.g., substrates including a metal or magnetic material).

As used herein, the term “polymer” refers to macromolecules having one or more structurally unique repeating units. The repeating units are referred to as “monomers,” which are polymerized for the polymer. Typically, a polymer is formed by monomers linked in a chain-like structure. A polymer formed entirely from a single type of monomer is referred to as a “homopolymer.” A polymer formed from two or more unique repeating structural units may be referred to as a “copolymer.” A polymer may be linear or branched, and may be random, block, polymer brush, hyperbranched polymer, bottlebrush polymer, dendritic polymer, or polymer micelles. The term “polymer” includes homopolymers, copolymers, tripolymers, tetra polymers and other polymeric molecules made from monomeric subunits. Copolymers include alternating copolymers, periodic copolymers, statistical copolymers, random copolymers, block copolymers, linear copolymers and branched copolymers. The term “polymerizable monomer” is used in accordance with its meaning in the art of polymer chemistry and refers to a compound that may covalently bind chemically to other monomer molecules (such as other polymerizable monomers that are the same or different) to form a polymer. Polymers can be hydrophilic, hydrophobic, or amphiphilic, as known in the art. Thus, “hydrophilic polymers” are substantially miscible with water and include, but are not limited to, polyethylene glycol and the like. “Hydrophobic polymers” are substantially immiscible with water and include, but are not limited to, polyethylene, polypropylene, polybutadiene, polystyrene, polymers disclosed herein, and the like. “Amphiphilic polymers” have both hydrophilic and hydrophobic properties and are typically copolymers having hydrophilic segment(s) and hydrophobic segment(s). Polymers include homopolymers, random copolymers, and block copolymers, as known in the art. The term “homopolymer” refers, in the usual and customary sense, to a polymer having a single monomeric unit. The term “copolymer” refers to a polymer derived from two or more monomeric species. The term “random copolymer” refers to a polymer derived from two or more monomeric species with no preferred ordering of the monomeric species. The term “block copolymer” refers to polymers having two or homopolymer subunits linked by covalent bond. Thus, the term “hydrophobic homopolymer” refers to a homopolymer which is hydrophobic. The term “hydrophobic block copolymer” refers to two or more homopolymer subunits linked by covalent bonds and which is hydrophobic.

As used herein, the term “hydrogel” refers to a three-dimensional polymeric structure that is substantially insoluble in water, but which is capable of absorbing and retaining large quantities of water to form a substantially stable, often soft and pliable, structure. In embodiments, water can penetrate in between polymer chains of a polymer network, subsequently causing swelling and the formation of a hydrogel. In embodiments, hydrogels are super-absorbent (e.g., containing more than about 90% water) and can be included of natural or synthetic polymers.

The term “surface” is intended to mean an external part or external layer of a substrate. The surface can be in contact with another material such as a gas, liquid, gel, polymer, organic polymer, second surface of a similar or different material, metal, or coating. The surface, or regions thereof, can be substantially flat. The substrate and/or the surface can have surface features such as wells, pits, channels, ridges, raised regions, pegs, posts or the like.

As used herein, the terms “cluster” and “colony” are used interchangeably to refer to a discrete site on a solid support that includes a plurality of immobilized polynucleotides and a plurality of immobilized complementary polynucleotides. The term “clustered array” refers to an array formed from such clusters or colonies. In this context the term “array” is not to be understood as requiring an ordered arrangement of clusters. The term “array” is used in accordance with its ordinary meaning in the art, and refers to a population of different molecules that are attached to one or more solid-phase substrates such that the different molecules can be differentiated from each other according to their relative location. An array can include different molecules that are each located at different addressable features on a solid-phase substrate. The molecules of the array can be nucleic acid primers, nucleic acid probes, nucleic acid templates or nucleic acid enzymes such as polymerases or ligases. Arrays useful in the invention can have densities that ranges from about 2 different features to many millions, billions or higher. The density of an array can be from 2 to as many as a billion or more different features per square cm. For example an array can have at least about 100 features/cm², at least about 1,000 features/cm², at least about 10,000 features/cm², at least about 100,000 features/cm², at least about 10,000,000 features/cm², at least about 100,000,000 features/cm², at least about 1,000,000,000 features/cm², at least about 2,000,000,000 features/cm² or higher. In embodiments, the arrays have features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

As used herein, the term “template polynucleotide” or “template nucleic acid” refers to any polynucleotide molecule that may be bound by a polymerase and utilized as a template for nucleic acid synthesis. A template polynucleotide may be a target polynucleotide. In general, the term “target polynucleotide” refers to a nucleic acid molecule or polynucleotide in a starting population of nucleic acid molecules having a target sequence whose presence, amount, and/or nucleotide sequence, or changes in one or more of these, are desired to be determined. In general, the term “target sequence” refers to a nucleic acid sequence on a single strand of nucleic acid. The terms “single strand” and “ssDNA” are used in accordance with its plain and ordinary meaning and refer to a single-stranded polynucleotide. The target sequence may be a portion of a gene, a regulatory sequence, genomic DNA, cDNA, RNA including mRNA, miRNA, rRNA, or others. The target sequence may be a target sequence from a sample or a secondary target such as a product of an amplification reaction. A target polynucleotide is not necessarily any single molecule or sequence. For example, a target polynucleotide may be any one of a plurality of target polynucleotides in a reaction, or all polynucleotides in a given reaction, depending on the reaction conditions. For example, in a nucleic acid amplification reaction with random primers, all polynucleotides in a reaction may be amplified. As a further example, a collection of targets may be simultaneously assayed using polynucleotide primers directed to a plurality of targets in a single reaction. As yet another example, all or a subset of polynucleotides in a sample may be modified by the addition of a primer-binding sequence (such as by the ligation of adapters containing the primer binding sequence), rendering each modified polynucleotide a target polynucleotide in a reaction with the corresponding primer polynucleotide(s). In the context of selective sequencing, “target polynucleotide(s)” refers to the subset of polynucleotide(s) to be sequenced from within a starting population of polynucleotides.

In embodiments, a target polynucleotide is a cell-free polynucleotide. In general, the terms “cell-free,” “circulating,” and “extracellular” as applied to polynucleotides (e.g. “cell-free DNA” (cfDNA) and “cell-free RNA” (cfRNA)) are used interchangeably to refer to polynucleotides present in a sample from a subject or portion thereof that can be isolated or otherwise manipulated without applying a lysis step to the sample as originally collected (e.g., as in extraction from cells or viruses). Cell-free polynucleotides are thus unencapsulated or “free” from the cells or viruses from which they originate, even before a sample of the subject is collected. Cell-free polynucleotides may be produced as a byproduct of cell death (e.g. apoptosis or necrosis) or cell shedding, releasing polynucleotides into surrounding body fluids or into circulation. Accordingly, cell-free polynucleotides may be isolated from a non-cellular fraction of blood (e.g. serum or plasma), from other bodily fluids (e.g. urine), or from non-cellular fractions of other types of samples.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

As used herein, the terms “analogue” and “analog”, in reference to a chemical compound, refers to compound having a structure similar to that of another one, but differing from it in respect of one or more different atoms, functional groups, or substructures that are replaced with one or more other atoms, functional groups, or substructures. In the context of a nucleotide, a nucleotide analog refers to a compound that, like the nucleotide of which it is an analog, can be incorporated into a nucleic acid molecule (e.g., an extension product) by a suitable polymerase, for example, a DNA polymerase in the context of a nucleotide analogue. The terms also encompass nucleic acids containing known nucleotide analogs or modified backbone residues or linkages, which are synthetic, naturally occurring, or non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see, e.g., see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

As used herein, a “native” nucleotide is used in accordance with its plain and ordinary meaning and refers to a naturally occurring nucleotide that does not include an exogenous label (e.g., a fluorescent dye, or other label) or chemical modification such as may characterize a nucleotide analog (e.g., a reversible terminating moiety). Examples of native nucleotides useful for carrying out procedures described herein include: dATP (2′-deoxyadenosine-5′-triphosphate); dGTP (2′-deoxyguanosine-5′-triphosphate); dCTP (2′-deoxycytidine-5′-triphosphate); dTTP (2′-deoxythymidine-5′-triphosphate); and dUTP (2′-deoxyuridine-5′-triphosphate). A “canonical” nucleotide is an unmodified nucleotide.

As used herein, the term “modified nucleotide” refers to nucleotide modified in some manner. Typically, a nucleotide contains a single 5-carbon sugar moiety, a single nitrogenous base moiety and 1 to three phosphate moieties. In embodiments, a nucleotide can include a blocking moiety (alternatively referred to herein as a reversible terminator moiety) and/or a label moiety. A blocking moiety on a nucleotide prevents formation of a covalent bond between the 3′ hydroxyl moiety of the nucleotide and the 5′ phosphate of another nucleotide. A blocking moiety on a nucleotide can be reversible, whereby the blocking moiety can be removed or modified to allow the 3′ hydroxyl to form a covalent bond with the 5′ phosphate of another nucleotide. A blocking moiety can be effectively irreversible under particular conditions used in a method set forth herein. In embodiments, the blocking moiety is attached to the 3′ oxygen of the nucleotide and is independently —NH₂, —CN, —CH₃, C₂-C₆ allyl (e.g., —CH₂—CH═CH₂), methoxyalkyl (e.g., —CH₂—O—CH₃), or —CH₂N₃. In embodiments, the blocking moiety is attached to the 3′ oxygen of the nucleotide and is independently

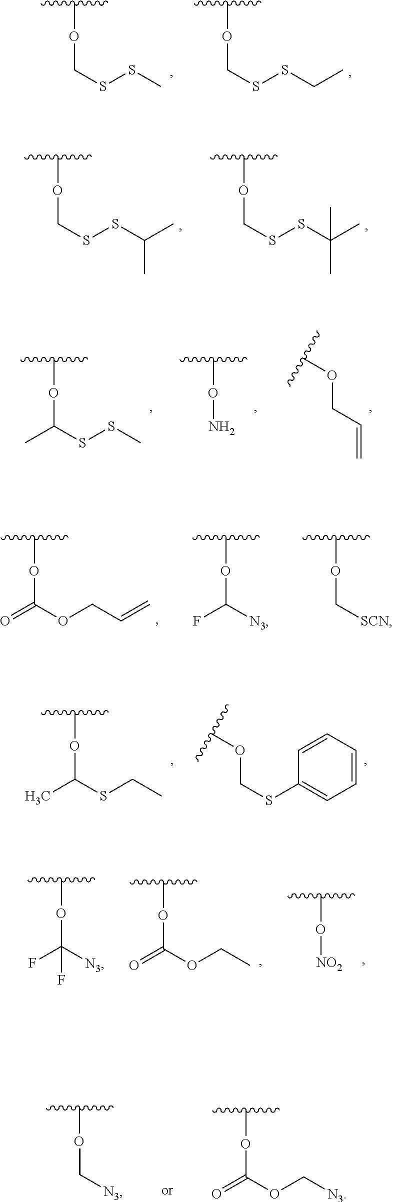

A label moiety of a nucleotide can be any moiety that allows the nucleotide to be detected, for example, using a spectroscopic method. Exemplary label moieties are fluorescent labels, mass labels, chemiluminescent labels, electrochemical labels, detectable labels and the like. One or more of the above moieties can be absent from a nucleotide used in the methods and compositions set forth herein. For example, a nucleotide can lack a label moiety or a blocking moiety or both. Examples of nucleotide analogues include, without limitation, 7-deaza-adenine, 7-deaza-guanine, the analogues of deoxynucleotides shown herein, analogues in which a label is attached through a cleavable linker to the 5-position of cytosine or thymine or to the 7-position of deaza-adenine or deaza-guanine, and analogues in which a small chemical moiety is used to cap the OH group at the 3′-position of deoxyribose. Nucleotide analogues and DNA polymerase-based DNA sequencing are also described in U.S. Pat. No. 6,664,079, which is incorporated herein by reference in its entirety for all purposes.

The term “cleavable linker” or “cleavable moiety” as used herein refers to a divalent or monovalent, respectively, moiety which is capable of being separated (e.g., detached, split, disconnected, hydrolyzed, a stable bond within the moiety is broken) into distinct entities. A cleavable linker is cleavable (e.g., specifically cleavable) in response to external stimuli (e.g., enzymes, nucleophilic/basic reagents, reducing agents, photo-irradiation, electrophilic/acidic reagents, organometallic and metal reagents, or oxidizing reagents). A chemically cleavable linker refers to a linker which is capable of being split in response to the presence of a chemical (e.g., acid, base, oxidizing agent, reducing agent, Pd(0), tris-(2-carboxyethyl)phosphine, dilute nitrous acid, fluoride, tris(3-hydroxypropyl)phosphine), sodium dithionite (Na₂S₂O₄), or hydrazine (N₂H₄)). A chemically cleavable linker is non-enzymatically cleavable. In embodiments, the cleavable linker is cleaved by contacting the cleavable linker with a cleaving agent. In embodiments, the cleaving agent is a phosphine containing reagent (e.g., TCEP or THPP), sodium dithionite (Na₂S₂O₄), weak acid, hydrazine (N₂H₄), Pd(0), or light-irradiation (e.g., ultraviolet radiation). In embodiments, cleaving includes removing. A “cleavable site” or “scissile linkage” in the context of a polynucleotide is a site which allows controlled cleavage of the polynucleotide strand (e.g., the linker, the primer, or the polynucleotide) by chemical, enzymatic, or photochemical means known in the art and described herein. A scissile site may refer to the linkage of a nucleotide between two other nucleotides in a nucleotide strand (i.e., an internucleosidic linkage). In embodiments, the scissile linkage can be located at any position within the one or more nucleic acid molecules, including at or near a terminal end (e.g., the 3′ end of an oligonucleotide) or in an interior portion of the one or more nucleic acid molecules. In embodiments, conditions suitable for separating a scissile linkage include a modulating the pH and/or the temperature. In embodiments, a scissile site can include at least one acid-labile linkage. For example, an acid-labile linkage may include a phosphoramidate linkage. In embodiments, a phosphoramidate linkage can be hydrolysable under acidic conditions, including mild acidic conditions such as trifluoroacetic acid and a suitable temperature (e.g., 30° C.), or other conditions known in the art, for example Matthias Mag, et al. Tetrahedron Letters, Volume 33, Issue 48, 1992, 7319-7322. In embodiments, the scissile site can include at least one photolabile internucleosidic linkage (e.g., o-nitrobenzyl linkages, as described in Walker et al., J. Am. Chem. Soc. 1988, 110, 21, 7170-7177), such as o-nitrobenzyloxymethyl or p-nitrobenzyloxymethyl group(s). In embodiments, the scissile site includes at least one uracil nucleobase. In embodiments, a uracil nucleobase can be cleaved with a uracil DNA glycosylase (UDG) or Formamidopyrimidine DNA Glycosylase Fpg. In embodiments, the scissile linkage site includes a sequence-specific nicking site having a nucleotide sequence that is recognized and nicked by a nicking endonuclease enzyme or a uracil DNA glycosylase.

As used herein, the term “loop” is used in accordance with its plain ordinary meaning and refers to the single-stranded region of a hairpin adapter that is located between the duplexed “stem” region of the hairpin adapter. In embodiments, the hairpin loop region is between about 4 nucleotides to 150 nucleotides in length. In embodiments, the hairpin loop is at least 4, 5, 6, 7, 8, 9, 10, 15, 20, 25, 30, 35, 40, 45, 50, or more nucleotides in length. In embodiments, the hairpin loop includes 1, 2, 3, 4, 5, 6, 7, 8, 9, 10, or more T nucleotides. In embodiments, the hairpin loop may include one or more of a primer binding sequence, a barcode, a UMI sequence, or a cleavable site. In some embodiments, a hairpin adapter includes a nucleic acid having a 5′-end, a 5′-portion, a loop, a 3′-portion and a 3′-end (e.g., arranged in a 5′ to 3′ orientation). In some embodiments, the 5′ portion of a hairpin adapter is annealed and/or hybridized to the 3′ portion of the hairpin adapter, thereby forming a stem portion of the hairpin adapter. In some embodiments, the 5′ portion of a hairpin adapter is substantially complementary to the 3′ portion of the hairpin adapter. In certain embodiments, a hairpin adapter includes a stem portion (i.e., stem) and a loop, wherein the stem portion is substantially double stranded thereby forming a duplex. In some embodiments, the loop of a hairpin adapter includes a nucleic acid strand that is not complementary (e.g., not substantially complementary) to itself or to any other portion of the hairpin adapter.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site blast.ncbi.nlm.nih.gov/Blast.cgi or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

As used herein, the term “removable” group, e.g., a label or a blocking group or protecting group, is used in accordance with its plain and ordinary meaning and refers to a chemical group that can be removed from a nucleotide analogue such that a DNA polymerase can extend the nucleic acid (e.g., a primer or extension product) by the incorporation of at least one additional nucleotide. Removal may be by any suitable method, including enzymatic, chemical, or photolytic cleavage. Removal of a removable group, e.g., a blocking group, does not require that the entire removable group be removed, only that a sufficient portion of it be removed such that a DNA polymerase can extend a nucleic acid by incorporation of at least one additional nucleotide using a nucleotide or nucleotide analogue. In general, the conditions under which a removable group is removed are compatible with a process employing the removable group (e.g., an amplification process or sequencing process).

As used herein, the terms “reversible blocking groups” and “reversible terminators” are used in accordance with their plain and ordinary meanings and refer to a blocking moiety located, for example, at the 3′ position of the nucleotide and may be a chemically cleavable moiety such as an allyl group, an azidomethyl group or a methoxymethyl group, or may be an enzymatically cleavable group such as a phosphate ester. Non-limiting examples of nucleotide blocking moieties are described in applications WO 2004/018497, U.S. Pat. Nos. 7,057,026, 7,541,444, WO 96/07669, U.S. Pat. Nos. 5,763,594, 5,808,045, 5,872,244 and 6,232,465 the contents of which are incorporated herein by reference in their entirety. Additional examples of a reversible terminator may be found in U.S. Pat. Nos. 6,664,079; 6,214,987; 5,872,244; Ju J. et al. (2006) Proc Natl Acad Sci USA 103(52):19635-19640; Ruparel H. et al. (2005) Proc Natl Acad Sci USA 102(17):5932-5937; Wu J. et al. (2007) Proc Natl Acad Sci USA 104(104):16462-16467; Guo J. et al. (2008) Proc Natl Acad Sci USA 105(27): 9145-9150 Bentley D. R. et al. (2008) Nature 456(7218):53-59; or Hutter D. et al. (2010) Nucleosides Nucleotides & Nucleic Acids 29:879-895, which are incorporated herein by reference in their entirety for all purposes. The nucleotides may be labelled or unlabeled. They may be modified with reversible terminators useful in methods provided herein and may be 3′-O-blocked reversible or 3′-unblocked reversible terminators. In nucleotides with 3′-O-blocked reversible terminators, the blocking group —OR [reversible terminating (capping) group] is linked to the oxygen atom of the 3′-OH of the pentose, while the label is linked to the base, which acts as a reporter and can be cleaved. The 3′-O-blocked reversible terminators are known in the art, and may be, for instance, a 3′-ONH₂ reversible terminator, a 3′-O-allyl reversible terminator, or a 3′-O-azidomethyl reversible terminator. In embodiments, the reversible terminator moiety is

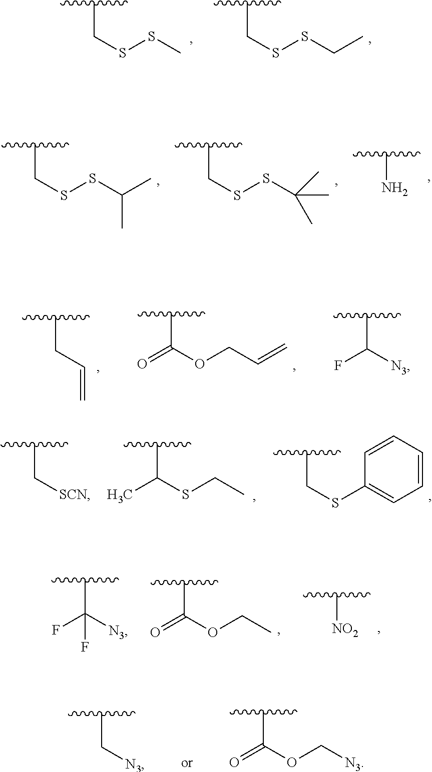

The term “allyl” as described herein refers to an unsubstituted methylene attached to a vinyl group (i.e., —CH═CH₂), having the formula

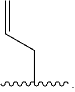 [SMILES: *CC=C]

In embodiments, the reversible terminator moiety is

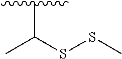 [SMILES: *C(C)SSC]

as described in U.S. Pat. No. 10,738,072, which is incorporated herein by reference for all purposes. For example, a nucleotide including a reversible terminator moiety may be represented by the formula:

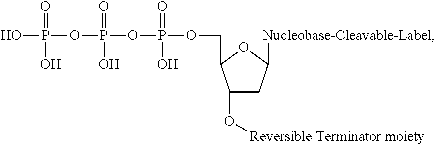

where the nucleobase is adenine or adenine analogue, thymine or thymine analogue, guanine or guanine analogue, or cytosine or cytosine analogue. In embodiments, the reversible terminator includes a hydrocarbyl. In embodiments, the reversible terminator includes an ester (O—C(O)R₁′ wherein R₁′ is any alkyl or aryl group which can include a formate, benzoyl formate, acetate, substituted acetate, propionate, and other esters as described in Green, T. W. (Protective Groups in Organic Chemistry, Wiley & Sons, New York, 1981)). In embodiments, the reversible terminator includes an ether (O—R₂′ wherein R₂′ can be substituted or unsubstituted alkyl such as methyl, substituted methyl, ethyl, substituted ethyl, allyl, substituted benzyl, silyl, or any other ether used to transiently protect hydroxyls and similar groups). In embodiments, the reversible terminator includes an O—CH₂(OC₂H₅)_{N′}CH₃ wherein N′ is an integer from 1-10. In embodiments, the reversible terminator includes a phosphate, phosphoramidate, phosphoramide, toluic acid ester, benzoic ester, acetic acid ester, or ethoxyethyl ether.

In some embodiments, a nucleic acid includes a molecular identifier or a molecular barcode. As used herein, the term “molecular barcode” (which may be referred to as a “tag”, a “barcode”, a “molecular identifier”, an “identifier sequence”, an “index”, or a “unique molecular identifier” (UMI)) refers to any material (e.g., a nucleotide sequence, a nucleic acid molecule feature) that is capable of distinguishing an individual molecule in a large heterogeneous population of molecules. In embodiments, a barcode is unique in a pool of barcodes that differ from one another in sequence, or is uniquely associated with a particular sample polynucleotide in a pool of sample polynucleotides. In embodiments, every barcode in a pool of adapters is unique, such that sequencing reads including the barcode can be identified as originating from a single sample polynucleotide molecule on the basis of the barcode alone. In other embodiments, individual barcode sequences may be used more than once, but adapters including the duplicate barcodes are associated with different sequences and/or in different combinations of barcoded adapters, such that sequence reads may still be uniquely distinguished as originating from a single sample polynucleotide molecule on the basis of a barcode and adjacent sequence information (e.g., sample polynucleotide sequence, and/or one or more adjacent barcodes). In embodiments, barcodes are about or at least about 5, 6, 7, 8, 9, 10, 15, 20, 25, 30, 40, 50, 75 or more nucleotides in length. In embodiments, barcodes are shorter than 20, 15, 10, 9, 8, 7, 6, or 5 nucleotides in length. In embodiments, barcodes are about 10 to about 50 nucleotides in length, such as about 15 to about 40 or about 20 to about 30 nucleotides in length. In a pool of different barcodes, barcodes may have the same or different lengths. In general, barcodes are of sufficient length and include sequences that are sufficiently different to allow the identification of sequencing reads that originate from the same sample polynucleotide molecule. In embodiments, each barcode in a plurality of barcodes differs from every other barcode in the plurality by at least three nucleotide positions, such as at least 3, 4, 5, 6, 7, 8, 9, 10, or more nucleotide positions. In some embodiments, substantially degenerate barcodes may be known as random.

As used herein, the terms “denaturant” or plural “denaturants” are used in accordance with their plain and ordinary meanings and refer to an additive or condition that disrupts the base pairing between nucleotides within opposing strands of a double-stranded polynucleotide molecule. The term “denature” and its variants, when used in reference to any double-stranded polynucleotide molecule, or double-stranded polynucleotide sequence, includes any process whereby the base pairing between nucleotides within opposing strands of the double-stranded molecule, or double-stranded sequence, is disrupted. Typically, denaturation includes rendering at least some portion or region of two strands of the double-stranded polynucleotide molecule or sequence single-stranded or partially single-stranded. In some embodiments, denaturation includes separation of at least some portion or region of two strands of the double-stranded polynucleotide molecule or sequence from each other. Typically, the denatured region or portion is then capable of hybridizing to another polynucleotide molecule or sequence. Optionally, there can be “complete” or “total” denaturation of a double-stranded polynucleotide molecule or sequence. Complete denaturation conditions are, for example, conditions that would result in complete separation of a significant fraction (e.g., more than 10%, 20%, 30%, 40% or 50%) of a large plurality of strands from their extended and/or full-length complements. Typically, complete or total denaturation disrupts all of the base pairing between the nucleotides of the two strands with each other. Similarly, a nucleic acid sample is optionally considered fully denatured when more than 80% or 90% of individual molecules of the sample lack any double-strandedness (or lack any hybridization to a complementary strand).

Optionally, a nucleic acid sample can be considered to be partially denatured when a substantial fraction of individual nucleic acid molecules of the sample (e.g., above 20%, 30%, 50%, or 70%) are in a partially denatured state. Optionally less than a substantial amount of individual nucleic acid molecules in the sample are fully denatured, e.g., not more than 5%, 10%, 20%, 30% or 50% of the nucleic acid molecules in the sample. Under exemplary conditions at least 50% of the nucleic acid molecules of the sample are partly denatured, but less than 20% or 10% are fully denatured. In other situations, at least 30% of the nucleic acid molecules of the sample are partly denatured, but less than 10% or 5% are fully denatured. Similarly, a nucleic acid sample can be non-denatured when a minority of individual nucleic acid molecules in the sample are partially or completely denatured.

In an embodiment, partially denaturing conditions are achieved by maintaining the duplexes as a suitable temperature range. For example, the nucleic acid is maintained at temperature sufficiently elevated to achieve some heat-denaturation (e.g., above 45° C., 50° C., 55° C., 60° C., 65° C., or 70° C.) but not high enough to achieve complete heat-denaturation (e.g., below 95° C. or 90° C. or 85° C. or 80° C. or 75° C.). In an embodiment the nucleic acid is partially denatured using substantially isothermal conditions. Alternatively, chemical denaturation can be accomplished by contacting the double-stranded polynucleotide to be denatured with appropriate chemical denaturants, such as strong alkalis, strong acids, chaotropic agents, and the like and can include, for example, NaOH, urea, or guanidine-containing compounds. In some embodiments, partial or complete denaturation is achieved by exposure to chemical denaturants such as urea or formamide, with concentrations suitably adjusted, or using high or low pH (e.g., pH between 4-6 or 8-9). In embodiments, the denaturant is a buffered solution including betaine, dimethyl sulfoxide (DMSO), ethylene glycol, formamide, glycerol, guanidine thiocyanate, 4-methylmorpholine 4-oxide (NMO), or a mixture thereof. In embodiments, the first denaturant is a buffered solution including about 0% to about 50% dimethyl sulfoxide (DMSO); about 0% to about 50% ethylene glycol; about 0% to about 20% formamide; or about 0 to about 3M betaine, or a mixture thereof. In an embodiment herein, partial denaturation and/or amplification, including any one or more steps or methods described herein, can be achieved using a recombinase and/or single-stranded binding protein.

In some embodiments, complete or partial denaturation of a double-stranded polynucleotide sequence is accomplished by contacting the double-stranded polynucleotide sequence using appropriate denaturing agents. For example, the double-stranded polynucleotide can be subjected to heat-denaturation (also referred to interchangeably as thermal denaturation) by raising the temperature to a point where the desired level of denaturation is accomplished. In some embodiments, thermal denaturation of a double-stranded polynucleotide, includes adjusting the temperature to achieve complete separation of the two strands of the polynucleotide, such that 90% or greater of the strands are in single-stranded form across their entire length. A completely denatured double-stranded polynucleotide results in a separated first strand and a second strand, each of which is a single-stranded polynucleotide. In some embodiments, complete thermal denaturation of a polynucleotide molecule (or polynucleotide sequence) is accomplished by exposing the polynucleotide molecule (or sequence) to a temperature that is at least 5° C., 10° C., 15° C., 20° C., 25° C., 30° C., 50° C., or 100° C., above the calculated or predict melting temperature (Tm) of the polynucleotide molecule or sequence.

In some embodiments, complete or partial denaturation is accomplished by treating the double-stranded polynucleotide sequence to be denatured using a denaturant mixture including an SSB protein (e.g., T4 gp32 protein, T7 gene 2.5 SSB protein, or phi29 SSB protein, Thermococcus kodakarensis (KOD) SSB, Thermus thermophilus (TTH) SSB, Sulfolobus solfataricus (SSO) SSB, or Extreme Thermostable Single-Stranded DNA Binding Protein (ET-SSB)), a strand-displacing polymerase (e.g., Bst large fragment (Bst LF) polymerase, Bst 3.0 polymerase, Bst 2.0 polymerase, Bsu polymerase, SD polymerase, Vent exo-polymerase, Phi29 polymerase, or a mutant thereof), and one or more crowding agents (poly(ethylene glycol) (PEG), polyvinylpyrrolidone (PVP), bovine serum albumin (BSA), dextran, Ficoll (e.g., Ficoll 70 or Ficoll 400), glycerol, or a combination thereof). In embodiments, the crowding agent is poly(ethylene glycol) (e.g., PEG 200, PEG 600, PEG 800, PEG 2,050, PEG 4,600, PEG 6,000, PEG 8,000, PEG 10,000, PEG 20,000, or PEG 35,000), dextran sulfate, bovine pancreatic trypsin inhibitor (BPTI), ribonuclease A, lysozyme, β-lactoglobulin, hemoglobin, bovine serum albumin (BSA), or poly(sodium 4-styrene sulfonate) (PSS). In embodiments, the denaturant mixture including an SSB, a strand-displacing polymerase, and one or more crowding agents does not include a chemical denaturant (e.g., betaine, DMSO, ethylene glycol, formamide, guanidine thiocyanate, NMO, TMAC, or a mixture thereof).

In some embodiments, a nucleic acid includes a label. As used herein, the term “label” or “labels” are used in accordance with their plain and ordinary meanings and refer to molecules that can directly or indirectly produce or result in a detectable signal either by themselves or upon interaction with another molecule. Non-limiting examples of detectable labels include fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the label is a dye. In embodiments, the dye is a fluorescent dye. Non-limiting examples of dyes, some of which are commercially available, include CF dyes (Biotium, Inc.), Alexa Fluor dyes (Thermo Fisher), DyLight dyes (Thermo Fisher), Cy dyes (GE Healthscience), IRDyes (Li-Cor Biosciences, Inc.), and HiLyte dyes (Anaspec, Inc.). In embodiments, a particular nucleotide type is associated with a particular label, such that identifying the label identifies the nucleotide with which it is associated. In embodiments, the label is luciferin that reacts with luciferase to produce a detectable signal in response to one or more bases being incorporated into an elongated complementary strand, such as in pyrosequencing. In embodiment, a nucleotide includes a label (such as a dye). In embodiments, the label is not associated with any particular nucleotide, but detection of the label identifies whether one or more nucleotides having a known identity were added during an extension step (such as in the case of pyrosequencing).

In embodiments, the detectable label is a fluorescent dye. In embodiments, the detectable label is a fluorescent dye capable of exchanging energy with another fluorescent dye (e.g., fluorescence resonance energy transfer (FRET) chromophores). Examples of detectable agents include imaging agents, including fluorescent and luminescent substances, including, but not limited to, a variety of organic or inorganic small molecules commonly referred to as “dyes,” “labels,” or “indicators.” Examples include fluorescein, rhodamine, acridine dyes, Alexa dyes, and cyanine dyes. In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent molecule (e.g., acridine dye, cyanine, dye, fluorine dye, oxazine dye, phenanthridine dye, or rhodamine dye). In embodiments, the detectable moiety is a moiety of a derivative of one of the detectable moieties described immediately above, wherein the derivative differs from one of the detectable moieties immediately above by a modification resulting from the conjugation of the detectable moiety to a compound described herein.

The term “cyanine” or “cyanine moiety” as described herein refers to a detectable moiety containing two nitrogen groups separated by a polymethine chain. In embodiments, the cyanine moiety has 3 methine structures (i.e., cyanine 3 or Cy3). In embodiments, the cyanine moiety has 5 methine structures (i.e., cyanine 5 or Cy5). In embodiments, the cyanine moiety has 7 methine structures (i.e., cyanine 7 or Cy7).

As used herein, the term “DNA polymerase” and “nucleic acid polymerase” are used in accordance with their plain ordinary meanings and refer to enzymes capable of synthesizing nucleic acid molecules from nucleotides (e.g., deoxyribonucleotides). Typically, a DNA polymerase adds nucleotides to the 3′-end of a DNA strand, one nucleotide at a time. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ι DNA polymerase, Pol κ DNA polymerase, Pol ζ DNA polymerase, Pol γ DNA polymerase, Pol θ DNA polymerase, Pol ν DNA polymerase, or a thermophilic nucleic acid polymerase (e.g. Therminator γ, 9° N polymerase (exo-), Therminator II, Therminator III, or Therminator IX). In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044). In embodiments, the polymerase is an enzyme described in US 2021/0139884. For example, a polymerase catalyzes the addition of a next correct nucleotide to the 3′-OH group of the primer via a phosphodiester bond, thereby chemically incorporating the nucleotide into the primer. Optionally, the polymerase used in the provided methods is a processive polymerase. Optionally, the polymerase used in the provided methods is a distributive polymerase.

As used herein, the term “exonuclease activity” is used in accordance with its ordinary meaning in the art, and refers to the removal of a nucleotide from a nucleic acid by a DNA polymerase. For example, during polymerization, nucleotides are added to the 3′ end of the primer strand. Occasionally a DNA polymerase incorporates an incorrect nucleotide to the 3′-OH terminus of the primer strand, wherein the incorrect nucleotide cannot form a hydrogen bond to the corresponding base in the template strand. Such a nucleotide, added in error, is removed from the primer as a result of the 3′ to 5′ exonuclease activity of the DNA polymerase. In embodiments, exonuclease activity may be referred to as “proofreading.” When referring to 3′-5′ exonuclease activity, it is understood that the DNA polymerase facilitates a hydrolyzing reaction that breaks phosphodiester bonds at the 3′ end of a polynucleotide chain to excise the nucleotide. In embodiments, 3′-5′ exonuclease activity refers to the successive removal of nucleotides in single-stranded DNA in a 3′→5′ direction, releasing deoxyribonucleoside 5′-monophosphates one after another. Methods for quantifying exonuclease activity are known in the art, see for example Southworth et al., PNAS Vol 93, 8281-8285 (1996). In embodiments, 5′-3′ exonuclease activity refers to the successive removal of nucleotides in double-stranded DNA in a 5′→3′ direction. In embodiments, the 5′-3′ exonuclease is lambda exonuclease. For example, lambda exonuclease catalyzes the removal of 5′ mononucleotides from duplex DNA, with a preference for 5′ phosphorylated double-stranded DNA. In other embodiments, the 5′-3′ exonuclease is E. coli DNA Polymerase I.

As used herein, the term “incorporating” or “chemically incorporating,” when used in reference to a primer and a nucleotide, refers to the process of joining the nucleotide to the primer or extension product thereof by formation of a phosphodiester bond.

As used herein, the term “selective” or “selectivity” or the like of a compound refers to the compound's ability to discriminate between molecular targets. When used in the context of sequencing, such as in “selectively sequencing,” this term refers to sequencing one or more target polynucleotides from an original starting population of polynucleotides, and not sequencing non-target polynucleotides from the starting population. Typically, selectively sequencing one or more target polynucleotides involves differentially manipulating the target polynucleotides based on known sequence. For example, target polynucleotides may be hybridized to a probe oligonucleotide that may be labeled (such as with a member of a binding pair) or bound to a surface. In embodiments, hybridizing a target polynucleotide to a probe oligonucleotide includes the step of displacing one strand of a double-stranded nucleic acid. Probe-hybridized target polynucleotides may then be separated from non-hybridized polynucleotides, such as by removing probe-bound polynucleotides from the starting population or by washing away polynucleotides that are not bound to a probe. The result is a selected subset of the starting population of polynucleotides, which is then subjected to sequencing, thereby selectively sequencing the one or more target polynucleotides.

As used herein, the terms “specific”, “specifically”, “specificity”, or the like of a compound refers to the agent's ability to cause a particular action, such as binding, to a particular molecular target with minimal or no action to other proteins in the cell.

As used herein, the terms “bind” and “bound” are used in accordance with their plain and ordinary meanings and refer to an association between atoms or molecules. The association can be direct or indirect. For example, bound atoms or molecules may be directly bound to one another, e.g., by a covalent bond or non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like)). As a further example, two molecules may be bound indirectly to one another by way of direct binding to one or more intermediate molecules, thereby forming a complex.

As used herein, the term “rolling circle amplification (RCA)” refers to a nucleic acid amplification reaction that amplifies a circular nucleic acid template (e.g., single-stranded DNA circles) via a rolling circle mechanism. Rolling circle amplification reaction is initiated by the hybridization of a primer to a circular, often single-stranded, nucleic acid template. The nucleic acid polymerase then extends the primer that is hybridized to the circular nucleic acid template by continuously progressing around the circular nucleic acid template to replicate the sequence of the nucleic acid template over and over again (rolling circle mechanism). The rolling circle amplification typically produces concatemers including tandem repeat units of the circular nucleic acid template sequence. The rolling circle amplification may be a linear RCA (LRCA), exhibiting linear amplification kinetics (e.g., RCA using a single specific primer), or may be an exponential RCA (ERCA) exhibiting exponential amplification kinetics. Rolling circle amplification may also be performed using multiple primers (multiply primed rolling circle amplification or MPRCA) leading to hyper-branched concatemers. For example, in a double-primed RCA, one primer may be complementary, as in the linear RCA, to the circular nucleic acid template, whereas the other may be complementary to the tandem repeat unit nucleic acid sequences of the RCA product. Consequently, the double-primed RCA may proceed as a chain reaction with exponential (geometric) amplification kinetics featuring a ramifying cascade of multiple-hybridization, primer-extension, and strand-displacement events involving both the primers. This often generates a discrete set of concatemeric, double-stranded nucleic acid amplification products. The rolling circle amplification may be performed in-vitro under isothermal conditions using a suitable nucleic acid polymerase such as Phi29 DNA polymerase. RCA may be performed by using any of the DNA polymerases that are known in the art (e.g., a Phi29 DNA polymerase, a Bst DNA polymerase, or SD polymerase).

As used herein, the terms “sequencing”, “sequence determination”, “determining a nucleotide sequence”, and the like include determination of a partial or complete sequence information, including the identification, ordering, or locations of the nucleotides that include the polynucleotide being sequenced, and inclusive of the physical processes for generating such sequence information. That is, the term includes sequence comparisons, consensus sequence determination, contig assembly, fingerprinting, and like levels of information about a target polynucleotide, as well as the express identification and ordering of nucleotides in a target polynucleotide. The term also includes the determination of the identification, ordering, and locations of one, two, or three of the four types of nucleotides within a target polynucleotide. In some embodiments, a sequencing process described herein includes contacting a template and an annealed primer with a suitable polymerase under conditions suitable for polymerase extension and/or sequencing. The sequencing methods are preferably carried out with the target polynucleotide arrayed on a solid substrate. Multiple target polynucleotides can be immobilized on the solid support through linker molecules, or can be attached to particles, e.g., microspheres, which can also be attached to a solid substrate. In embodiments, the solid substrate is in the form of a chip, a bead, a well, a capillary tube, a slide, a wafer, a filter, a fiber, a porous media, or a column. In embodiments, the solid substrate is gold, quartz, silica, plastic, glass, diamond, silver, metal, or polypropylene. In embodiments, the solid substrate is porous.

As used herein, the term “sequencing cycle” is used in accordance with its plain and ordinary meaning and refers to incorporating one or more nucleotides (e.g., nucleotide analogues) to the 3′ end of a polynucleotide with a polymerase, and detecting the one or more nucleotides incorporated. In embodiments, one nucleotide (e.g., a modified nucleotide) is incorporated per sequencing cycle. The sequencing may be accomplished by, for example, sequencing by synthesis, pyrosequencing, and the like. In embodiments, a sequencing cycle includes extending a complementary polynucleotide by incorporating a first nucleotide using a polymerase, wherein the polynucleotide is hybridized to a template nucleic acid, detecting the first nucleotide, and identifying the first nucleotide. An “extension strand” is formed as the one or more nucleotides are incorporated into a complementary polynucleotide hybridized to a template nucleic acid. The extension strand is complementary to the template nucleic acid. In embodiments, to begin a sequencing cycle, one or more differently labeled nucleotides and a DNA polymerase can be introduced. Following nucleotide addition, signals produced (e.g., via excitation and emission of a detectable label) can be detected to determine the identity of the incorporated nucleotide (based on the labels on the nucleotides). Reagents can then be added to remove the 3′ reversible terminator and to remove labels from each incorporated base. Reagents, enzymes, and other substances can be removed between steps by washing. Cycles may include repeating these steps, and the sequence of each cluster is read over the multiple repetitions.

As used herein, the term “sequencing reaction mixture” is used in accordance with its plain and ordinary meaning and refers to an aqueous mixture that contains the reagents sufficient to allow a dNTP or dNTP analogue to add a nucleotide to a DNA strand by a DNA polymerase. In embodiments, the sequencing reaction mixture includes a buffer. In embodiments, the buffer includes an acetate buffer, 3-(N-morpholino) propanesulfonic acid (MOPS) buffer, N-(2-Acetamido)-2-aminoethanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(Cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer. In embodiments, the sequencing reaction mixture includes nucleotides, wherein the nucleotides include a reversible terminating moiety and a label covalently linked to the nucleotide via a cleavable linker. In embodiments, the sequencing reaction mixture includes a buffer, DNA polymerase, detergent (e.g., Triton X), a chelator (e.g., EDTA), or salts (e.g., ammonium sulfate, magnesium chloride, sodium chloride, or potassium chloride).

As used herein, the term “extension” or “elongation” is used in accordance with their plain and ordinary meanings and refer to synthesis by a polymerase of a new polynucleotide strand (i.e., an “extension strand”) complementary to a template strand by adding free nucleotides (e.g., dNTPs) from a reaction mixture that are complementary to the template in a 5′-to-3′ direction, including condensing a 5′-phosphate group of a dNTPs with a 3′-hydroxy group at the end of the nascent (elongating) DNA strand.

As used herein, the term “sequencing read” is used in accordance with its plain and ordinary meaning and refers to an inferred sequence of nucleotide bases (or nucleotide base probabilities) corresponding to all or part of a single polynucleotide fragment. A sequencing read may include 10, 20, 30, 40, 50, 60, 70, 80, 90, 100, 150, 200, 250, or more nucleotide bases. In embodiments, a sequencing read includes reading a barcode and a template nucleotide sequence. In embodiments, a sequencing read includes reading a template nucleotide sequence. In embodiments, a sequencing read includes reading a barcode and not a template nucleotide sequence. In embodiments, a sequencing read includes a computationally derived string corresponding to the detected label. The sequence reads are optionally stored in an appropriate data structure for further evaluation. In embodiments, a first sequencing reaction can generate a first sequencing read. The first sequencing read can provide the sequence of a first region of the polynucleotide fragment. In embodiments, a second sequencing primer can initiate sequencing at a second location on the nucleic acid template. The second location can be distinct from the first location. In some cases, a 3′ terminal nucleotide of the second primer can hybridize to a location that is more than 5 nucleotides away from a binding site of a 3′ terminal nucleotide of the first primer. The second sequencing reaction can generate a second sequencing read. The second sequencing read can provide the sequence of a second region of the nucleic acid template which is distinct from the first region of the nucleic acid template. In some embodiments, the nucleic acid template is optionally subjected to one or more additional rounds of sequencing using additional sequencing primers, thereby generating additional sequencing reads. In embodiments, a sequencing read is about 25 nucleotide bases. In embodiments, a sequencing read is about 35 nucleotide bases. In embodiments, a sequencing read is about 45 nucleotide bases. In embodiments, a sequencing read is about 55 nucleotide bases. In embodiments, a sequencing read is about 65 nucleotide bases. In embodiments, a sequencing read is about 75 nucleotide bases. In embodiments, a sequencing read is about 85 nucleotide bases. In embodiments, a sequencing read is a string of characters representing the sequence of nucleotides. In embodiments, the length of a sequencing read corresponds to the length of the target sequence. In embodiments, the length of a sequencing read corresponds to the number of sequencing cycles. A sequencing read may be subjected to initial processing (often termed “pre-processing”) prior to annotation. Pre-processing includes filtering out low-quality sequences, sequence trimming to remove continuous low-quality nucleotides, merging paired-end sequences, or identifying and filtering out PCR repeats using known techniques in the art. The sequenced reads may then be assembled and aligned using bioinformatic algorithms known in the art. A sequencing read may be aligned to a reference sequence. In embodiments, a sequencing read includes a sequence corresponding to an interposing oligonucleotide probe sequence (e.g., a sequencing primer binding sequence of the interposing oligonucleotide probe). In embodiments, a sequencing read includes a computationally derived string corresponding to the detected complementary nucleotide (e.g., a labeled nucleotide). The sequence reads are optionally stored in an appropriate data structure for further evaluation. In embodiments, a first sequencing reaction can generate a first sequencing read. The first sequencing read can provide the sequence of a first region of the polynucleotide fragment. In some embodiments, the nucleic acid template is optionally subjected to one or more additional rounds of sequencing using additional sequencing primers, thereby generating additional sequencing reads.

The term “multiplexing” as used herein refers to an analytical method in which the presence and/or amount of multiple targets, e.g., multiple nucleic acid target sequences, can be assayed simultaneously by using the methods and devices as described herein, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic.

As used herein, the term “hybridize” or “specifically hybridize” refers to a process where two complementary nucleic acid strands anneal to each other under appropriately stringent conditions. Hybridizations are typically and preferably conducted with oligonucleotides. The terms “annealing” and “hybridization” are used interchangeably to mean the formation of a stable duplex. In some embodiments, one portion of a nucleic acid hybridizes to itself, such as in the formation of a hairpin structure. The propensity for hybridization between nucleic acids depends on the temperature and ionic strength of their milieu, the length of the nucleic acids and the degree of complementarity. The effect of these parameters on hybridization is described in, for example, Sambrook J., Fritsch E. F., Maniatis T., Molecular cloning: a laboratory manual, Cold Spring Harbor Laboratory Press, New York (1989). As used herein, hybridization of a primer, or of a DNA extension product, respectively, is extendable by creation of a phosphodiester bond with an available nucleotide or nucleotide analogue capable of forming a phosphodiester bond, therewith. For example, hybridization can be performed at a temperature ranging from 15° C. to 95° C. In some embodiments, the hybridization is performed at a temperature of about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C. In other embodiments, the stringency of the hybridization can be further altered by the addition or removal of components of the buffered solution. Those skilled in the art understand how to estimate and adjust the stringency of hybridization conditions such that sequences having at least a desired level of complementarity will stably hybridize, while those having lower complementarity will not. As used herein, the term “stringent condition” refers to condition(s) under which a polynucleotide probe or primer will hybridize preferentially to its target sequence, and to a lesser extent to, or not at all to, other sequences. In some embodiments nucleic acids, or portions thereof, that are configured to specifically hybridize are often about 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more or 100% complementary to each other over a contiguous portion of nucleic acid sequence. A specific hybridization discriminates over non-specific hybridization interactions (e.g., two nucleic acids that a not configured to specifically hybridize, e.g., two nucleic acids that are 80% or less, 70% or less, 60% or less or 50% or less complementary) by about 2-fold or more, often about 10-fold or more, and sometimes about 100-fold or more, 1000-fold or more, 10,000-fold or more, 100,000-fold or more, or 1,000,000-fold or more. Two nucleic acid strands (e.g., two single-stranded polynucleotides) that are hybridized to each other can form a duplex which includes a double-stranded portion of nucleic acid.

A nucleic acid can be amplified by a suitable method. The term “amplification,” “amplified” or “amplifying” as used herein refers to subjecting a target nucleic acid in a sample to a process that linearly or exponentially generates amplicon nucleic acids having the same or substantially the same (e.g., substantially identical) nucleotide sequence as the target nucleic acid, or segment thereof, and/or a complement thereof. In some embodiments an amplification reaction includes a suitable thermal stable polymerase. Thermal stable polymerases are known in the art and are stable for prolonged periods of time, at temperature greater than 80° C. when compared to common polymerases found in most mammals. In certain embodiments the term “amplified” refers to a method that includes a polymerase chain reaction (PCR). Conditions conducive to amplification (i.e., amplification conditions) are known and often include at least a suitable polymerase, a suitable template, a suitable primer or set of primers, suitable nucleotides (e.g., dNTPs), a suitable buffer, and application of suitable annealing, hybridization and/or extension times and temperatures. In certain embodiments an amplified product (e.g., an amplicon) can contain one or more additional and/or different nucleotides than the template sequence, or portion thereof, from which the amplicon was generated (e.g., a primer can contain “extra” nucleotides (such as a 5′ portion that does not hybridize to the template), or one or more mismatched bases within a hybridizing portion of the primer).

As used herein, bridge-PCR (bPCR) amplification is a method for solid-phase amplification as exemplified by the disclosures of U.S. Pat. Nos. 5,641,658; 7,115,400; and U.S. Patent Publ. No. 2008/0009420, each of which is incorporated herein by reference in its entirety. Bridge-PCR involves repeated polymerase chain reaction cycles, cycling between denaturation, annealing, and extension conditions and enables controlled, spatially-localized, amplification, to generate amplification products (e.g., amplicons) immobilized on a solid support in order to form arrays comprised of colonies (or “clusters”) of immobilized nucleic acid molecule.

Amplification according to the present teachings encompasses any means by which at least a part of at least one target nucleic acid is reproduced, typically in a template-dependent manner, including without limitation, a broad range of techniques for amplifying nucleic acid sequences, either linearly or exponentially. Illustrative means for performing an amplifying step include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), and the like, including multiplex versions and combinations thereof, for example but not limited to, OLA (oligonucleotide ligation assay)/PCR, PCR/OLA, LDR/PCR, PCR/PCR/LDR, PCR/LDR, LCR/PCR, PCR/LCR (also known as combined chain reaction—CCR), and the like. Descriptions of such techniques can be found in, among other sources, Ausbel et al.; PCR Primer: A Laboratory Manual, Diffenbach, Ed., Cold Spring Harbor Press (1995); The Electronic Protocol Book, Chang Bioscience (2002); Msuih et al., J. Clin. Micro. 34:501-07 (1996); The Nucleic Acid Protocols Handbook, R. Rapley, ed., Humana Press, Totowa, N.J. (2002); Abramson et al., Curr Opin Biotechnol. 1993 February; 4(1):41-7, U.S. Pat. Nos. 6,027,998; 6,605,451, Barany et al., PCT Publication No. WO 97/31256; Wenz et al., PCT Publication No. WO 01/92579; Day et al., Genomics, 29(1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin, Development of a Multiplex Ligation Detection Reaction DNA Typing Assay, Sixth International Symposium on Human Identification, 1995 (available on the world wide web at: promega.com/geneticidproc/ussymp6proc/blegrad.html-); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27:e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18-(2002); Lage et al., Genome Res. 2003 February; 13(2):294-307, and Landegren et al., Science 241:1077-80 (1988), Demidov, V., Expert Rev Mol Diagn. 2002 November; 2(6):542-8., Cook et al., J Microbiol Methods. 2003 May; 53(2):165-74, Schweitzer et al., Curr Opin Biotechnol. 2001 February; 12(1):21-7, U.S. Pat. Nos. 5,830,711, 6,027,889, 5,686,243, PCT Publication No. WO0056927A3, and PCT Publication No. WO9803673A1.

A nucleic acid can be amplified by a thermocycling method or by an isothermal amplification method. In some embodiments, a rolling circle amplification method is used. In some embodiments, amplification takes place on a solid support (e.g., within a flow cell) where a nucleic acid, nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support.

In some embodiments solid phase amplification includes a nucleic acid amplification reaction including only one species of oligonucleotide primer immobilized to a surface or substrate. In certain embodiments solid phase amplification includes a plurality of different immobilized oligonucleotide primer species. In some embodiments solid phase amplification may include a nucleic acid amplification reaction including one species of oligonucleotide primer immobilized on a solid surface and a second different oligonucleotide primer species in solution. Multiple different species of immobilized or solution-based primers can be used. Non-limiting examples of solid phase nucleic acid amplification reactions include interfacial amplification, bridge amplification, emulsion PCR, WildFire amplification (e.g., US patent publication US 2013/0012399), the like or combinations thereof.

Provided herein are methods and compositions for analyzing a sample (e.g., sequencing nucleic acids within a sample). A sample (e.g., a sample including nucleic acid) can be obtained from a suitable subject. A sample can be isolated or obtained directly from a subject or part thereof. In some embodiments, a sample is obtained indirectly from an individual or medical professional. A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, ear, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. A fluid or tissue sample from which nucleic acid is extracted may be acellular (e.g., cell-free). Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, ear, nails, the like, parts thereof or combinations thereof. A sample may include cells or tissues that are normal, healthy, diseased (e.g., infected), and/or cancerous (e.g., cancer cells). A sample obtained from a subject may include cells or cellular material (e.g., nucleic acids) of multiple organisms (e.g., virus nucleic acid, fetal nucleic acid, bacterial nucleic acid, parasite nucleic acid).

In some embodiments, a sample includes nucleic acid, or fragments thereof. A sample can include nucleic acids obtained from one or more subjects. In some embodiments a sample includes nucleic acid obtained from a single subject. In some embodiments, a sample includes a mixture of nucleic acids. A mixture of nucleic acids can include two or more nucleic acid species having different nucleotide sequences, different fragment lengths, different origins (e.g., genomic origins, cell or tissue origins, subject origins, the like or combinations thereof), or combinations thereof. A sample may include synthetic nucleic acid.

A subject can be any living or non-living organism, including but not limited to a human, non-human animal, plant, bacterium, fungus, virus or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a human subject. A subject can be a patient (e.g., a human patient). In some embodiments a subject is suspected of having a genetic variation or a disease or condition associated with a genetic variation.

The methods and kits of the present disclosure may be applied, mutatis mutandis, to the sequencing of RNA, or to determining the identity of a ribonucleotide.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., packaging, buffers, written instructions for performing a method, etc.) from one location to another. For example, kits include one or more enclosures (e.g., boxes) containing the relevant reaction reagents and/or supporting materials. As used herein, the term “fragmented kit” refers to a delivery system including two or more separate containers that each contain a subportion of the total kit components. The containers may be delivered to the intended recipient together or separately. For example, a first container may contain an enzyme for use in an assay, while a second container contains oligonucleotides. In contrast, a “combined kit” refers to a delivery system containing all of the components of a reaction assay in a single container (e.g., in a single box housing each of the desired components). The term “kit” includes both fragmented and combined kits.

The terms “bioconjugate group,” “bioconjugate reactive moiety,” and “bioconjugate reactive group” refer to a chemical moiety which participates in a reaction to form a bioconjugate linker (e.g., covalent linker). Non-limiting examples of bioconjugate reactive groups and the resulting bioconjugate reactive linkers may be found in the Bioconjugate Table below:

| Bioconjugate reactive group 1 | Bioconjugate reactive group 2 | Resulting |
|---|---|---|
| (e.g., electrophilic bioconjugate | (e.g., nucleophilic bioconjugate | Bioconjugate |
| reactive moiety) | reactive moiety) | reactive linker |
| activated esters | amines/anilines | carboxamides |
| acrylamides | thiols | thioethers |
| acyl azides | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides |
| aryl halides | thiols | thiophenols |
| aryl halides | amines | aryl amines |
| aziridines | thiols | thioethers |
| boronates | glycols | boronate esters |
| carbodiimides | carboxylic acids | N-acylureas or anhydrides |
| diazoalkanes | carboxylic acids | esters |
| epoxides | thiols | thioethers |
| haloacetamides | thiols | thioethers |
| haloplatinate | amino | platinum complex |
| haloplatinate | heterocycle | platinum complex |
| haloplatinate | thiol | platinum complex |
| halotriazines | amines/anilines | aminotriazines |
| halotriazines | alcohols/phenols | triazinyl ethers |
| halotriazines | thiols | triazinyl thioethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonate esters | thiols | thioethers |
| sulfonate esters | carboxylic acids | esters |
| sulfonate esters | alcohols | ethers |
| sulfonyl halides | amines/anilines | sulfonamides |
| sulfonyl halides | phenols/alcohols | sulfonate esters |

As used herein, the term “bioconjugate reactive moiety” and “bioconjugate reactive group” refers to a moiety or group capable of forming a bioconjugate (e.g., covalent linker) as a result of the association between atoms or molecules of bioconjugate reactive groups. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like)). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine). Useful bioconjugate reactive groups used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; (o) biotin conjugate can react with avidin or streptavidin to form a avidin-biotin complex or streptavidin-biotin complex.

The term “covalent linker” is used in accordance with its ordinary meaning and refers to a divalent moiety which connects at least two moieties to form a molecule. The term “non-covalent linker” is used in accordance with its ordinary meaning and refers to a divalent moiety which includes at least two molecules that are not covalently linked to each other but are capable of interacting with each other via a non-covalent bond (e.g., electrostatic interactions (e.g., ionic bond, hydrogen bond, halogen bond) or van der Waals interactions (e.g., dipole-dipole, dipole-induced dipole, London dispersion)). In embodiments, the non-covalent linker is the result of two molecules that are not covalently linked to each other that interact with each other via a non-covalent bond.

The term “adapter” as used herein refers to any linear oligonucleotide that can be ligated to a nucleic acid molecule, thereby generating nucleic acid products that can be sequenced on a sequencing platform (e.g., an Illumina or Singular Genomics G4™ sequencing platform). In embodiments, adapters include two reverse complementary oligonucleotides forming a double-stranded structure. In embodiments, an adapter includes two oligonucleotides that are complementary at one portion and mismatched at another portion, forming a Y-shaped or fork-shaped adapter that is double stranded at the complementary portion and has two overhangs at the mismatched portion. Since Y-shaped adapters have a complementary, double-stranded region, they can be considered a special form of double-stranded adapters. When this disclosure contrasts Y-shaped adapters and double stranded adapters, the term “double-stranded adapter” or “blunt-ended” is used to refer to an adapter having two strands that are fully complementary, substantially (e.g., more than 90% or 95%) complementary, or partially complementary. In embodiments, adapters include sequences that bind to sequencing primers. In embodiments, adapters include sequences that bind to immobilized oligonucleotides (e.g., P7 and P5 sequences) or reverse complements thereof. In embodiments, the adapter is substantially non-complementary to the 3′ end or the 5′ end of any target polynucleotide present in the sample. In embodiments, the adapter can include a sequence that is substantially identical, or substantially complementary, to at least a portion of a primer, for example a universal primer. In embodiments, the adapter can include an index sequence (also referred to as barcode or tag) to assist with downstream error correction, identification or sequencing.

Where a range of values is provided, it is understood that each intervening value, to the tenth of the unit of the lower limit unless the context clearly indicates otherwise, between the upper and lower limit of that range, and any other stated or unstated intervening value in, or smaller range of values within, that stated range is encompassed within the invention. The upper and lower limits of any such smaller range (within a more broadly recited range) may independently be included in the smaller ranges, or as particular values themselves, and are also encompassed within the invention, subject to any specifically excluded limit in the stated range. Where the stated range includes one or both of the limits, ranges excluding either or both of those included limits are also included in the invention.

The term “nucleic acid sequencing device” and the like means an integrated system of one or more chambers, ports, and channels that are interconnected and in fluid communication and designed for carrying out an analytical reaction or process, either alone or in cooperation with an appliance or instrument that provides support functions, such as sample introduction, fluid and/or reagent driving means, temperature control, detection systems, data collection and/or integration systems, for the purpose of determining the nucleic acid sequence of a template polynucleotide. Nucleic acid sequencing devices may further include valves, pumps, and specialized functional coatings on interior walls. Nucleic acid sequencing devices may include a receiving unit, or platen, that orients the flow cell such that a maximal surface area of the flow cell is available to be exposed to an optical lens. Other nucleic acid sequencing devices include those provided by Singular Genomics™ (e.g., the G4™ system), Illumina™ (e.g., HiSeq™ MiSeq™, NextSeq™, or NovaSeq™ systems), Life Technologies™ (e.g., ABI PRISM™, or SOLiD™ systems), Pacific Biosciences (e.g., systems using SMRT™ Technology such as the Sequel™ or RS II™ systems), or Qiagen (e.g., Genereader™ system). Nucleic acid sequencing devices may further include fluidic reservoirs (e.g., bottles), valves, pressure sources, pumps, sensors, control systems, valves, pumps, and specialized functional coatings on interior walls. In embodiments, the device includes a plurality of a sequencing reagent reservoirs and a plurality of clustering reagent reservoirs. In embodiments, the clustering reagent reservoir includes amplification reagents (e.g., an aqueous buffer containing enzymes, salts, and nucleotides, denaturants, crowding agents, etc.) In embodiments, the reservoirs include sequencing reagents (such as an aqueous buffer containing enzymes, salts, and nucleotides); a wash solution (an aqueous buffer); a cleave solution (an aqueous buffer containing a cleaving agent, such as a reducing agent); or a cleaning solution (a dilute bleach solution, dilute NaOH solution, dilute HCl solution, dilute antibacterial solution, or water). The fluid of each of the reservoirs can vary. The fluid can be, for example, an aqueous solution which may contain buffers (e.g., saline-sodium citrate (SSC), ascorbic acid, tris(hydroxymethyl)aminomethane or “Tris”), aqueous salts (e.g., KCl or (NH₄)₂SO₄)), nucleotides, polymerases, cleaving agent (e.g., tri-n-butyl-phosphine, triphenyl phosphine and its sulfonated versions (i.e., tris(3-sulfophenyl)-phosphine, TPPTS), and tri(carboxyethyl)phosphine (TCEP) and its salts, cleaving agent scavenger compounds (e.g., 2′-Dithiobisethanamine or 11-Azido-3,6,9-trioxaundecane-1-amine), chelating agents (e.g., EDTA), detergents, surfactants, crowding agents, or stabilizers (e.g., PEG, Tween, BSA). Non-limited examples of reservoirs include cartridges, pouches, vials, containers, and eppendorf tubes. In embodiments, the device is configured to perform fluorescent imaging. In embodiments, the device includes one or more light sources (e.g., one or more lasers). In embodiments, the illuminator or light source is a radiation source (i.e., an origin or generator of propagated electromagnetic energy) providing incident light to the sample. A radiation source can include an illumination source producing electromagnetic radiation in the ultraviolet (UV) range (about 200 to 390 nm), visible (VIS) range (about 390 to 770 nm), or infrared (IR) range (about 0.77 to 25 microns), or other range of the electromagnetic spectrum. In embodiments, the illuminator or light source is a lamp such as an arc lamp or quartz halogen lamp. In embodiments, the illuminator or light source is a coherent light source. In embodiments, the light source is a laser, LED (light emitting diode), a mercury or tungsten lamp, or a super-continuous diode. In embodiments, the light source provides excitation beams having a wavelength between 200 nm to 1500 nm. In embodiments, the laser provides excitation beams having a wavelength of 405 nm, 470 nm, 488 nm, 514 nm, 520 nm, 532 nm, 561 nm, 633 nm, 639 nm, 640 nm, 800 nm, 808 nm, 912 nm, 1024 nm, or 1500 nm. In embodiments, the illuminator or light source is a light-emitting diode (LED). The LED can be, for example, an Organic Light Emitting Diode (OLED), a Thin Film Electroluminescent Device (TFELD), or a Quantum dot based inorganic organic LED. The LED can include a phosphorescent OLED (PHOLED). In embodiments, the nucleic acid sequencing device includes an imaging system (e.g., an imaging system as described herein). The imaging system capable of exciting one or more of the identifiable labels (e.g., a fluorescent label) linked to a nucleotide and thereafter obtain image data for the identifiable labels. The image data (e.g., detection data) may be analyzed by another component within the device. The imaging system may include a system described herein and may include a fluorescence spectrophotometer including an objective lens and/or a solid-state imaging device. The solid-state imaging device may include a charge coupled device (CCD) and/or a complementary metal oxide semiconductor (CMOS). The system may also include circuitry and processors, including systems using microcontrollers, reduced instruction set computers (RISC), application specific integrated circuits (ASICs), field programmable gate array (FPGAs), logic circuits, and any other circuit or processor capable of executing functions described herein. The set of instructions may be in the form of a software program. As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. In embodiments, the device includes a thermal control assembly useful to control the temperature of the reagents.

As used herein, the term “upstream” refers to a region in the nucleic acid sequence that is towards the 5′ end of a particular reference point, and the term “downstream” refers to a region in the nucleic acid sequence that is toward the 3′ end of the reference point.

As used herein, the term “hairpin adapter” refers to a polynucleotide including a double-stranded stem portion and a single-stranded hairpin loop portion. In some embodiments, an adapter is a hairpin adapter (also referred to herein as a “hairpin”). In some embodiments, a hairpin adapter includes a single nucleic acid strand including a stem-loop structure. In some embodiments, a hairpin adapter includes a nucleic acid having a 5′-end, a 5′-portion, a loop, a 3′-portion and a 3′-end (e.g., arranged in a 5′ to 3′ orientation). In some embodiments, the 5′ portion of a hairpin adapter is annealed and/or hybridized to the 3′ portion of the hairpin adapter, thereby forming a stem portion of the hairpin adapter. In some embodiments, the 5′ portion of a hairpin adapter is substantially complementary to the 3′ portion of the hairpin adapter. In certain embodiments, a hairpin adapter includes a stem portion (i.e., stem) and a loop, wherein the stem portion is substantially double stranded thereby forming a duplex. In some embodiments, the loop of a hairpin adapter includes a nucleic acid strand that is not complementary (e.g., not substantially complementary) to itself or to any other portion of the hairpin adapter. In some embodiments, a method herein includes ligating a first adapter to a first end of a double stranded nucleic acid, and ligating a second adapter to a second end of a double stranded nucleic acid. In some embodiments, the first adapter and the second adapter are different. For example, in certain embodiments, the first adapter and the second adapter may include different nucleic acid sequences or different structures. In some embodiments, the first adapter is a Y-adapter and the second adapter is a hairpin adapter. In some embodiments, the first adapter is a hairpin adapter and a second adapter is a hairpin adapter. In certain embodiments, the first adapter and the second adapter may include different primer binding sites, different structures, and/or different capture sequences (e.g., a sequence complementary to a capture nucleic acid). In some embodiments, some, all or substantially all of the nucleic acid sequence of a first adapter and a second adapter are the same. In some embodiments, some, all or substantially all of the nucleic acid sequence of a first adapter and a second adapter are substantially different.

As used herein, “capable of hybridizing” is used in accordance with its ordinary meaning in the art and refers to two oligonucleotides that, under suitable conditions, can form a duplex (e.g., Watson-Crick pairing) which includes a double-stranded portion of nucleic acid. Such conditions, known in the art and described herein, depend upon, for example, the nature of the nucleotide sequence, temperature, and buffer conditions. The stringency of hybridization can be influenced by various parameters, including degree of identity and/or complementarity between the polynucleotides (or any target sequences within the polynucleotides) to be hybridized; melting point of the polynucleotides and/or target sequences to be hybridized, referred to as “Tm”; parameters such as salts, buffers, pH, temperature, GC % content of the polynucleotide and primers, and/or time. Typically, hybridization is favored in lower temperatures and/or increased salt concentrations, as well as reduced concentrations of organic solvents. Some exemplary conditions suitable for hybridization include incubation of the polynucleotides to be hybridized in solutions having sodium salts, such as NaCl, sodium citrate and/or sodium phosphate. In some embodiments, hybridization or wash solutions can include about 10-75% formamide and/or about 0.01-0.7% sodium dodecyl sulfate (SDS). In some embodiments, a hybridization solution can be a stringent hybridization solution which can include any combination of 50% formamide, 5×SSC (0.75 M NaCl, 0.075 M sodium citrate), 50 mM sodium phosphate (pH 6.8), 0.1% sodium pyrophosphate, 5×Denhardt's solution, 0.1% SDS, and/or 10% dextran sulfate. In some embodiments, the hybridization or washing solution can include BSA (bovine serum albumin). In some embodiments, hybridization or washing can be conducted at a temperature range of about 20-25° C., or about 25-30° C., or about 30-35° C., or about 35-40° C., or about 40-45° C., or about 45-50° C., or about 50-55° C., or higher. In some embodiments, hybridization or washing can be conducted for a time range of about 1-10 minutes, or about 10-20 minutes, or about 20-30 minutes, or about 30-40 minutes, or about 40-50 minutes, or about 50-60 minutes, or longer. In some embodiments, hybridization or wash conditions can be conducted at a pH range of about 5-10, or about pH 6-9, or about pH 6.5-8, or about pH 6.5-7.

As used herein, “hybridization complex” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

As used herein, the term “adjacent,” refers to two nucleotide sequences in a nucleic acid, can refer to nucleotide sequences separated by 0 to about 20 nucleotides, more specifically, in a range of about 1 to about 10 nucleotides, or to sequences that directly abut one another. As those of skill in the art appreciate, two nucleotide sequences that that are to ligated together will generally directly abut one another.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### II. Methods

In an aspect is provided a method of sequencing two or more different regions (e.g., three, four, five, six, seven, eight, nine, or 10 different regions) of a template nucleic acid. In embodiments, the method includes (a) contacting a first primer annealed to a first region of the template nucleic acid with a sequencing solution including a plurality of nucleotides and incorporating with a polymerase one or more nucleotides into the first primer to create a first extension strand, and detecting the one or more incorporated nucleotides so as to identify each incorporated nucleotide in the first extension strand; (b) contacting the template nucleic acid with a blocking element thereby terminating extension of the first extension strand thereby forming a blocked first extension strand; (c) contacting a second primer annealed to a second region of the template nucleic acid with a sequencing solution including a plurality of nucleotides and incorporating with a polymerase one or more nucleotides into the second primer to create a second extension strand, and detecting the one or more incorporated nucleotides so as to identify each incorporated nucleotide in the second extension strand; (d) contacting the template nucleic acid with a blocking element thereby terminating extension of the second extension strand and creating a blocked second extension strand; and (e) contacting a third primer annealed to a third region of the template nucleic acid with a sequencing solution including a plurality of nucleotides and incorporating with a polymerase one or more nucleotides into the third primer to create a third extension strand, and detecting the one or more incorporated nucleotides so as to identify each incorporated nucleotide in the third extension strand.

In another aspect is provided a method of sequencing a single-strand polynucleotide, the method including: (a) hybridizing a first sequencing primer to a first primer binding sequence of the single-strand polynucleotide and incorporating with a polymerase one or more nucleotides into the sequencing primer to create a first extension strand, and detecting the one or more incorporated nucleotides so as to identify each incorporated nucleotide in the first extension strand; and (b) contacting the first extension strand with a first blocking element thereby terminating extension of the first extension strand thereby forming a first blocked extension strand; (c) repeating steps (a) and (b), wherein each repetition of steps (a) and (b) includes hybridizing a different sequencing primer to a different primer binding sequence of the single-strand polynucleotide (e.g., a downstream sequence); and (d) hybridizing a terminal sequencing primer to a terminal primer binding sequence of the single-strand polynucleotide and incorporating with a polymerase one or more nucleotides into the terminal sequencing primer to create a terminal extension strand, and detecting the one or more incorporated nucleotides so as to identify each incorporated nucleotide in the extension strand. In embodiments, step (d) does not include contacting the terminal extension strand with a blocking element. In embodiments, step (d) occurs after step (c).

In embodiments, the single-stranded polynucleotide is 1 kb or greater. In embodiments, the single-stranded polynucleotide is 2 kb or greater. In embodiments, the single-stranded polynucleotide is 3 kb or greater. In embodiments, the single-stranded polynucleotide is 4 kb or greater. In embodiments, the single-stranded polynucleotide is 5 kb or greater. In embodiments, the single-stranded polynucleotide is 6 kb or greater. In embodiments, the single-stranded polynucleotide is 7 kb or greater. In embodiments, the single-stranded polynucleotide is 8 kb or greater. In embodiments, the single-stranded polynucleotide is 9 kb or greater. In embodiments, the single-stranded polynucleotide is 10 kb or greater. In embodiments, the single-stranded polynucleotide is about 1 kb. In embodiments, the single-stranded polynucleotide is about 2 kb. In embodiments, the single-stranded polynucleotide is about 3 kb. In embodiments, the single-stranded polynucleotide is about 4 kb. In embodiments, the single-stranded polynucleotide is about 5 kb. In embodiments, the single-stranded polynucleotide is about 6 kb. In embodiments, the single-stranded polynucleotide is about 7 kb. In embodiments, the single-stranded polynucleotide is about 8 kb. In embodiments, the single-stranded polynucleotide is about 9 kb. In embodiments, the single-stranded polynucleotide is about 10 kb. In embodiments, the single-stranded polynucleotide is about 20 kb. In embodiments, the single-stranded polynucleotide is about 30 kb. In embodiments, the single-stranded polynucleotide is about 40 kb. In embodiments, the single-stranded polynucleotide is about 50 kb. In embodiments, the single-stranded polynucleotide is about 1 kb to about 50 kb. In embodiments, the single-stranded polynucleotide is about 1 kb to about 5 kb. In embodiments, the single-stranded polynucleotide is about 1 kb to about 10 kb. In embodiments, the single-stranded polynucleotide is about 2 kb to about 10 kb. In embodiments, the single-stranded polynucleotide is about 2 kb to about 3 kb.

In embodiments, the single-stranded polynucleotide is about 100 to 1000 nucleotides in length. In embodiments, the single-stranded polynucleotide is about 350 nucleotides in length. In embodiments, the single-stranded polynucleotide is about 10, 20, 50, 100, 150, 200, 300, or 500 nucleotides in length. The single-stranded polynucleotide molecules can vary length, such as about 100-300 nucleotides long, about 300-500 nucleotides long, or about 500-1000 nucleotides long. In embodiments, the single-stranded polynucleotide molecular is about 100-1000 nucleotides, about 150-950 nucleotides, about 200-900 nucleotides, about 250-850 nucleotides, about 300-800 nucleotides, about 350-750 nucleotides, about 400-700 nucleotides, or about 450-650 nucleotides. In embodiments, the single-stranded polynucleotide molecule is about 150 nucleotides. In embodiments, the single-stranded polynucleotide is about 100-1000 nucleotides long. In embodiments, the single-stranded polynucleotide is about 100-300 nucleotides long. In embodiments, the single-stranded polynucleotide is about 300-500 nucleotides long. In embodiments, the single-stranded polynucleotide is about 500-1000 nucleotides long. In embodiments, the single-stranded polynucleotide molecule is about 100 nucleotides. In embodiments, the single-stranded polynucleotide molecule is about 300 nucleotides. In embodiments, the single-stranded polynucleotide molecule is about 500 nucleotides. In embodiments, the single-stranded polynucleotide molecule is about 1,000 nucleotides. In embodiments, the single-stranded polynucleotide molecule is about 2,000 nucleotides. In embodiments, the single-stranded polynucleotide molecule is about 3,000 nucleotides. In embodiments, the single-stranded polynucleotide molecule is about 4,000 nucleotides. In embodiments, the single-stranded polynucleotide molecule is about 5,000 nucleotides. In embodiments, the single-stranded polynucleotide molecule is about 6,000 nucleotides. In embodiments, the single-stranded polynucleotide molecule is about 7,000 nucleotides. In embodiments, the single-stranded polynucleotide molecule is about 8,000 nucleotides. In embodiments, the single-stranded polynucleotide molecule is about 9,000 nucleotides. In embodiments, the single-stranded polynucleotide molecule is about 10,000 nucleotides. In embodiments, the single-stranded polynucleotide molecule is about 10,000 to about 50,000 nucleotides. In embodiments, the single-stranded polynucleotide molecule is about 20,000 nucleotides. In embodiments, the single-stranded polynucleotide molecule is about 30,000 nucleotides. In embodiments, the single-stranded polynucleotide molecule is about 40,000 nucleotides. In embodiments, the single-stranded polynucleotide molecule is about 50,000 nucleotides.

In embodiments, steps (a) and (b) are repeated three or more times. In embodiments, each repetition of steps (a) and (b) is referred to as a seq-block cycle. In embodiments, each seq-block cycle generates a seq-block extension strand. In embodiments, the method includes 2, 3, 4, 5, 6, 7, 8, 9, or 10 seq-block cycles. In embodiments, the method includes at least 2 seq-block cycles. In embodiments, the method includes at least 3 seq-block cycles. In embodiments, the method includes at least 4 seq-block cycles.

In embodiments, contacting the extension strand with a blocking element includes hybridizing a blocking oligonucleotide downstream of the extension strand, wherein said blocking oligonucleotide includes locked nucleic acids (LNAs), Bis-locked nucleic acids (bisLNAs), twisted intercalating nucleic acids (TINAs), bridged nucleic acids (BNAs), 2′-O-methyl RNA:DNA chimeric nucleic acids, minor groove binder (MGB) nucleic acids, morpholino nucleic acids, C5-modified pyrimidine nucleic acids, peptide nucleic acids (PNAs), phosphorothioate nucleic acids, or combinations thereof.

In embodiments, each extension strand (e.g., the first extension strand, the terminal extension strand, or an extension strand of the seq-block cycle) is independently about 5 to about 200 nucleotides in length. In embodiments, each extension strand is substantially the same length (e.g., the first extension strand and subsequent seq-block extension strands are about 100 nucleotides in length). In embodiments, one or more of the extension strands are different lengths. In embodiments, one or more of the extension strands is 8-12 nucleotides in length, and one or more of the extension strands is 1-150 nucleotides in length. In embodiments, each extension strand is at least 50, 75, 100, 150, 175, 200, or more nucleotides in length.

In embodiments, the single-stranded polynucleotide includes three or more different sequencing primer binding sequences. In embodiments, the single-stranded polynucleotide is formed according to a method described herein. For example, in an aspect is provided a method of forming a single-stranded polynucleotide including three or more sequencing primer binding sequences. In embodiments, the single-stranded polynucleotide is formed by hybridizing two or more interposing oligonucleotide probes to a template nucleic acid molecule, wherein each of the interposing oligonucleotide probes includes from 5′ to 3′: i. a first hybridization sequence complementary to a first sequence of the template nucleic acid; ii. a loop region including a sequencing primer binding sequence; and iii. a second hybridization sequence complementary to a second sequence of the template nucleic acid; extending the 3′ end of each second hybridization sequence of the interposing oligonucleotide probes with one or more polymerases thereby forming an extension product of each of the oligonucleotide probes; and ligating the 3′ end of each of the extension products to the 5′ end of the adjacent extension products, thereby making an integrated strand including a complement of the template nucleic acid including a plurality of the oligonucleotide probes. In embodiments, the method further includes hybridizing a 5′ terminal oligonucleotide probe downstream of the one or more interposing oligonucleotide probes, wherein the 5′ terminal oligonucleotide probe includes from 5′ to 3′: i. a hybridization sequence complementary to a third sequence of the template nucleic acid; and ii. a primer binding sequence; and hybridizing a 3′ terminal oligonucleotide probe upstream of the one or more interposing oligonucleotide probes, wherein the 3′ terminal oligonucleotide probe includes from 3′ to 5′: i. a hybridization sequence complementary to a fourth sequence of the template nucleic acid; and ii. a primer binding sequence; extending the 3′ end of the hybridization sequence of the 3′ terminal oligonucleotide probe with one or more polymerases thereby forming an extension product; and ligating the 5′ end of the 5′ terminal oligonucleotide probe to the 3′ end of the adjacent extension product. In embodiments, the single-stranded polynucleotide is formed prior to step a. In embodiments, prior to step a) the method includes amplifying the single-stranded polynucleotide to generate a plurality of single-stranded polynucleotides.

In embodiments, the single-stranded polynucleotide is attached to a solid support. In embodiments, the single-stranded polynucleotide is attached to polymer, wherein the polymer is attached to the solid support. In embodiments, the single-stranded polynucleotide is covalently attached to a solid support. In embodiments, amplifying includes bridge polymerase chain reaction (bPCR) amplification, solid-phase rolling circle amplification (RCA), solid-phase exponential rolling circle amplification (eRCA), solid-phase recombinase polymerase amplification (RPA), or solid-phase helicase dependent amplification (HDA). In embodiments, amplifying includes bridge polymerase chain reaction (bPCR) amplification. In embodiments, amplifying includes isothermal amplification. In embodiments, amplifying includes solid-phase rolling circle amplification (RCA) or solid-phase exponential rolling circle amplification (eRCA).

In embodiments, amplifying includes forming a plurality of amplification products. In embodiments, forming a plurality of amplification products includes bridge polymerase chain reaction (bPCR) amplification, solid-phase rolling circle amplification (RCA), solid-phase exponential rolling circle amplification (eRCA), solid-phase recombinase polymerase amplification (RPA), solid-phase helicase dependent amplification (HDA), template walking amplification, or emulsion PCR on particles, or combinations of the methods. In embodiments, generating a double-stranded amplification product includes a bridge polymerase chain reaction (bPCR) amplification. In embodiments, generating a double-stranded amplification product includes a thermal bridge polymerase chain reaction (t-bPCR) amplification. In embodiments, generating a double-stranded amplification product includes a chemical bridge polymerase chain reaction (c-bPCR) amplification. Chemical bridge polymerase chain reactions include fluidically cycling a denaturant (e.g., formamide) and maintaining the temperature within a narrow temperature range (e.g., +/−5° C.). In contrast, thermal bridge polymerase chain reactions include thermally cycling between high temperatures (e.g., 85° C.-95° C.) and low temperatures (e.g., 60° C.-70° C.). Thermal bridge polymerase chain reactions may also include a denaturant, typically at a much lower concentration than traditional chemical bridge polymerase chain reactions.

In embodiments, forming a plurality of amplification products includes amplifying the template polynucleotide or complement thereof on a solid support including a plurality of primers attached to the solid support, wherein the plurality of primers include a plurality of forward primers with complementarity to the template polynucleotide and a plurality of reverse primers with complementarity to a complement of the template polynucleotide, and the amplifying includes a plurality of cycles of strand denaturation, primer hybridization, and primer extension.

In embodiments, the plurality of strand denaturation cycles are different for one or more cycles, wherein the initial denaturation cycle is maintained at different conditions from the remaining denaturation cycles. For example, in embodiments, the initial denaturation cycle is at about 85° C.-95° C. for about 1 minute to about 10 minutes, whereas denaturation in the remaining cycles is different (e.g., about 85° C. for about 15-30 sec). In embodiments, the initial denaturation is maintained at about 85° C.-95° C. for about 5 minutes to about 10 minutes. In embodiments, the initial denaturation is maintained at 90° C.-95° C. for about 1 to 10 minutes. In embodiments, the initial denaturation is maintained at 80° C.-85° C. for about 1 to 10 minutes. In embodiments, the initial denaturation is maintained at 85° C.-90° C. for about 1 to 10 minutes. In embodiments, the initial denaturation is maintained at about 85° C.-95° C. for about 1 minutes to about 10 minutes. In embodiments, the initial denaturation is maintained at about 95° C. for about 5 minutes to about 10 minutes. In embodiments, the initial denaturation is maintained at about 85° C.-95° C. for about 5 minutes to about 10 minutes.

In embodiments, forming a plurality of amplification products includes chemical bridge polymerase chain reaction (c-bPCR) amplification. In embodiments, forming a plurality of amplification products includes denaturation using a chemical denaturant. In embodiments, forming a plurality of amplification products includes denaturation using acetic acid, hydrochloric acid, nitric acid, formamide, guanidine, sodium salicylate, sodium hydroxide, dimethyl sulfoxide (DMSO), propylene glycol, urea, or a mixture thereof. In embodiments, the chemical denaturant is sodium hydroxide or formamide. In embodiments, forming a plurality of amplification products includes thermal bridge polymerase chain reaction (t-bPCR) amplification. In embodiments, forming a plurality of amplification products includes chemical bridge polymerase chain reaction (c-bPCR) amplification. Chemical bridge polymerase chain reactions include fluidically cycling a denaturant (e.g., formamide) and maintaining the temperature within a narrow temperature range (e.g., +/−5° C.). In contrast, thermal bridge polymerase chain reactions include thermally cycling between high temperatures (e.g., 85° C.-95° C.) and low temperatures (e.g., 60° C.-70° C.). Thermal bridge polymerase chain reactions may also include a denaturant, typically at a significantly lower concentration than traditional chemical bridge polymerase chain reactions.

In embodiments, forming a plurality of amplification products includes fluidic cycling between an extension mixture that includes a polymerase and dNTPs, and a chemical denaturant. In embodiments, the polymerase is a strand-displacing polymerase or a non-strand displacing polymerase. In embodiments, the solutions are thermally cycled between about 40° C. to about 65° C. during fluidic cycling of the extension mixture and the chemical denaturant. For example, the extension cycle is maintained at a temperature of 55° C.-65° C., followed by a denaturation cycle that is maintained at a temperature of 40° C.-65° C., or by a denaturation step in which the temperature starts at 60° C.-65° C. and is ramped down to 40° C. prior to exchanging the reagent. In embodiments, step (b) includes modulating the reaction temperature prior to initiating the next cycle. In embodiments, the denaturation cycle and/or the extension cycle is maintained at a temperature for a sufficient amount of time, and prior to starting the next cycle the temperature is modulated (e.g., increased relative to the starting temperature or reduced relative to the starting temperature). In embodiments, the denaturation cycle is performed at a temperature of 60° C.-65° C. for about 5-45 sec, then the temperature is reduced (e.g., lowered to about 40° C.) before starting an extension cycle (i.e., before introducing an extension mixture). Lowering the temperature, even in the presence of a chemical denaturant, facilitates primer hybridization in the subsequent step when the amplicons are exposed to conditions that promote hybridization. In embodiments, the extension cycle is performed at a temperature of 50° C.-60° C. for about 0.5-2 minutes, then the temperature is increased (e.g., raised to between about 60° C. to about 70° C., or to about 65° C. to about 72° C.) after introducing the extension mixture. In embodiments, the cycling between the extension mixture and the chemical denaturant is performed at least 5, at least 10, at least 20, at least 30, at least 40, at least 50, at least 75, at least 100, or at least 200 times. In embodiments, the cycling between the extension mixture and the chemical denaturant is performed about 5, about 10, about 20, about 30, about 40, about 50, about 75, about 100, or about 200 times. In embodiments, the cycling between the extension mixture and the chemical denaturant is performed a total of 5, 10, 20, 30, 40, 50, 75, 100, 200, or more times. In embodiments, the fluidic cycling is performed in the presence of about 2 to about 15 mM Mg2+. In embodiments, the fluidic cycling is performed in the presence of about 2, about 3, about 4, about 5, about 6, about 7, about 8, about 9, about 10, about 11, about 12, about 13, about 14, or about 15 mM Mg²⁺.

In embodiments, forming a plurality of amplification products includes a plurality of strand denaturation cycles, wherein the initial denaturation cycle is at different conditions from the remaining denaturation cycles. For example, in embodiments, the initial denaturation cycle is at about 85° C.-95° C. for about 1 minute to about 10 minutes, whereas denaturation in the remaining cycles is different (e.g. about 85° C. for about 15-30 sec). In embodiments, forming a plurality of amplification products includes an initial denaturation at about 85° C.-95° C. for about 5 minutes to about 10 minutes. In embodiments, forming a plurality of amplification products includes an initial denaturation at 90° C.-95° C. for about 1 to 10 minutes. In embodiments, forming a plurality of amplification products includes an initial denaturation at 80° C.-85° C. for about 1 to 10 minutes. In embodiments, forming a plurality of amplification products includes an initial denaturation at 85° C.-90° C. for about 1 to 10 minutes.

In embodiments, the plurality of cycles includes thermally cycling between (i) about 80° C. to 90° C. for denaturation, and (ii) about 55° C. to about 65° C. for annealing/extension of the primer. In embodiments, the plurality of cycles includes thermally cycling between (i) about 85° C. for denaturation, and (ii) about 55° C. for annealing/extension of the primer. In embodiments, the plurality of cycles includes thermally cycling between (i) about 85° C. for denaturation, and (ii) about 65° C. for annealing/extension of the primer. In embodiments, the plurality of cycles includes thermally cycling between (i) less than 80° C. (e.g., 70 to 80° C.) for denaturation, and (ii) about 55° C. to about 65° C. for annealing/extension of the primer. In embodiments, the plurality of cycles includes thermally cycling between (i) about 70° C. for denaturation, and (ii) about 65° C. for annealing/extension of the primer. In embodiments, the plurality of cycles includes thermally cycling between (i) about 75° C. for denaturation, and (ii) about 55° C. for annealing/extension of the primer. In embodiments, the plurality of cycles includes thermally cycling between (i) about 85° C. for denaturation, and (ii) about 65° C. for annealing/extension of the primer.

In embodiments, forming a plurality of amplification products includes incubation in a denaturant. In embodiments, the denaturant is acetic acid, ethylene glycol, hydrochloric acid, nitric acid, formamide, guanidine, sodium salicylate, sodium hydroxide, dimethyl sulfoxide (DMSO), propylene glycol, urea, or a mixture thereof. In embodiments, the denaturant is an additive that lowers a DNA denaturation temperature. In embodiments, the denaturant is betaine, dimethyl sulfoxide (DMSO), ethylene glycol, formamide, glycerol, guanidine thiocyanate, 4-methylmorpholine 4-oxide (NMO), or a mixture thereof. In embodiments, the denaturant is betaine, dimethyl sulfoxide (DMSO), ethylene glycol, formamide, glycerol, guanidine thiocyanate, or 4-methylmorpholine 4-oxide (NMO).

In embodiments, forming a plurality of amplification products includes a plurality of cycles of strand denaturation, primer hybridization, and primer extension. Although each cycle will include each of these three events (denaturation, hybridization, and extension), events within a cycle may or may not be discrete. For example, each step may have different reagents and/or reaction conditions (e.g., temperatures). Alternatively, some steps may proceed without a change in reaction conditions. For example, extension may proceed under the same conditions (e.g., same temperature) as hybridization. After extension, the conditions are changed to start a new cycle with a new denaturation step, thereby amplifying the amplicons. Primer extension products from an earlier cycle may serve as templates for a later amplification cycle. In embodiments, the plurality of cycles is about 5 to about 50 cycles. In embodiments, the plurality of cycles is about 10 to about 45 cycles. In embodiments, the plurality of cycles is about 10 to about 20 cycles. In embodiments, the plurality of cycles is about 20 to about 30 cycles. In embodiments, the plurality of cycles is 10 to 45 cycles. In embodiments, the plurality of cycles is 10 to 20 cycles. In embodiments, the plurality of cycles is 20 to 30 cycles. In embodiments, the plurality of cycles is about 10 to about 45 cycles. In embodiments, the plurality of cycles is about 20 to about 30 cycles.

In embodiments, forming a plurality of amplification products includes rolling circle amplification (RCA) (see, e.g., Lizardi et al., Nat. Genet. 19:225-232 (1998), which is incorporated herein by reference in its entirety). Several suitable RCA methods are known in the art. For example, RCA amplifies a circular polynucleotide (e.g., DNA) by polymerase extension of an amplification primer complementary to a portion of the template nucleic acid. This process generates copies of the circular polynucleotide template such that multiple complements of the template sequence arranged end to end in tandem are generated (i.e., a concatemer).

In embodiments, forming a plurality of amplification products includes exponential rolling circle amplification (eRCA). Exponential RCA is similar to the linear process except that it uses a second primer having a sequence that is identical to at least a portion of the circular template (Lizardi et al. Nat. Genet. 19:225 (1998)). This two-primer system achieves isothermal, exponential amplification. Exponential RCA has been applied to the amplification of non-circular DNA through the use of a linear probe that binds at both of its ends to contiguous regions of a target DNA followed by circularization using DNA ligase (Nilsson et al. Science 265(5181):208 5(1994)).

In embodiments, forming a plurality of amplification products includes hyperbranched rolling circle amplification (HRCA). Hyperbranched RCA uses a second primer complementary to the first amplification product. This allows products to be replicated by a strand-displacement mechanism, which can yield a drastic amplification within an isothermal reaction (Lage et al., Genome Research 13:294-307 (2003), which is incorporated herein by reference in its entirety).

In embodiments, the solid support includes a plurality of immobilized primers. In embodiments, the immobilized primers may be referred to as amplification primers. In embodiments, the amplification primers are each attached to the solid support (i.e., immobilized on the surface of a solid support). The polynucleotide molecules can be fixed to surface by a variety of techniques, including covalent attachment and non-covalent attachment. In embodiments, the polynucleotides are confined to an area of a discrete region (referred to as a cluster). The discrete regions may have defined locations in a regular array, which may correspond to a rectilinear pattern, circular pattern, hexagonal pattern, or the like. A regular array of such regions is advantageous for detection and data analysis of signals collected from the arrays during an analysis. These discrete regions are separated by interstitial regions. As used herein, the term “interstitial region” refers to an area in a substrate or on a surface that separates other areas of the substrate or surface. For example, an interstitial region can separate one concave feature of an array from another concave feature of the array. The two regions that are separated from each other can be discrete, lacking contact with each other. In another example, an interstitial region can separate a first portion of a feature from a second portion of a feature. In embodiments the interstitial region is continuous whereas the features are discrete, for example, as is the case for an array of wells in an otherwise continuous surface. The separation provided by an interstitial region can be partial or full separation. Interstitial regions will typically have a surface material that differs from the surface material of the features on the surface. For example, features of an array can have polynucleotides that exceeds the amount or concentration present at the interstitial regions. In some embodiments the polynucleotides and/or primers may not be present at the interstitial regions. In embodiments, at least two different primers are attached to the solid support (e.g., a forward and a reverse primer), which facilitates generating multiple amplification products from the first extension product or a complement thereof.

In embodiments, the amplification products are localized to sites (e.g., wells) on a solid support, which may be referred to as clusters following generation of a plurality of immobilized amplification products. In embodiments, the clusters have a mean or median separation from one another of about 0.5-5 μm. In embodiments, the mean or median separation is about 0.1-10 microns, 0.25-5 microns, 0.5-2 microns, 1 micron, or a number or a range between any two of these values. In embodiments, the mean or median separation is about or at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0 μm or a number or a range between any two of these values. In embodiments, the mean or median separation is about 0.1-10 microns. In embodiments, the mean or median separation is about 0.25-5 microns. In embodiments, the mean or median separation is about 0.5-2 microns. In embodiments, the mean or median separation is about or at least about 0.1 μm. In embodiments, the mean or median separation is about or at least about 0.25 μm. In embodiments, the mean or median separation is about or at least about 0.5 μm. In embodiments, the mean or median separation is about or at least about 1.0 μm. In embodiments, the mean or median separation is about or at least about 1.5 μm. In embodiments, the mean or median separation is about or at least about 2.0 μm. In embodiments, the mean or median separation is about or at least about 5.0 μm. In embodiments, the mean or median separation is about or at least about 10 μm. The mean or median separation may be measured center-to-center (i.e., the center of one cluster to the center of a second cluster). In embodiments of the methods provided herein, the amplicon clusters have a mean or median separation (measured center-to-center) from one another of about 0.5-5 μm. The mean or median separation may be measured edge-to-edge (i.e., the edge of one amplicon cluster to the edge of a second amplicon cluster). In embodiments of the methods provided herein, the amplicon clusters have a mean or median separation (measured edge-to-edge) from one another of about 0.2-5 μm. In embodiments, the mean or median separation is about or at least about 1.0, 1.1, 1.2, 1.3, 1.4, 1.5, 1.6, 1.7, 1.8, 1.9, or 2.0 μm. In embodiments, the mean or median separation is about 1.0, 1.1, 1.2, 1.3, 1.4, 1.5, 1.6, 1.7, 1.8, 1.9, or 2.0 μm.

In embodiments, amplifying is performed in an emulsion.

In embodiments, the first hybridization sequence, the second hybridization sequence, or both include one or more phosphorothioate-containing nucleotides or one or more LNAs. In embodiments, the first hybridization sequence includes one or more phosphorothioate-containing nucleotides or one or more LNAs. In embodiments, the second hybridization sequence includes one or more phosphorothioate-containing nucleotides or one or more LNAs.

In embodiments, the method includes producing a plurality of sequencing reads during each step (a); grouping sequencing reads based on co-occurrence of primer binding sequences; and within each group, computationally aligning the reads that belong to the same strand of an original sample polynucleotide based on the sequences of the primer binding sequences.

In embodiments, the blocked first extension strand is upstream of the blocked second extension strand, third extension strand, or both the blocked second extension strand and third extension strand.

In embodiments, the blocking element includes a chain-terminating nucleotide (e.g., a non-extendable nucleotide). In embodiments, contacting the template nucleic acid with a blocking element includes incorporating a chain-terminating nucleotide into the 3′ end of the extension strand. In embodiments, the chain-terminating nucleotide includes a ddNTP, a reversibly-terminated dNTP, or a modified nucleotide triphosphate which lacks a 3′-OH. In embodiments, the chain-terminating nucleotide includes a ddNTP, a reversibly-terminated dNTP, or any nucleotide triphosphate which lacks a 3′-OH. In embodiments, the chain-terminating nucleotide includes a ddNTP. In embodiments, the chain-terminating nucleotide includes a reversibly-terminating dNTP. In embodiments, the reversibly-terminating dNTP uses orthogonal chemistry to the chemistry used during sequencing (i.e., the reversible terminator of the reversibly-terminating dNTP may be removed independently of the reversible terminator using during sequencing). In embodiments, the chain-terminating nucleotide includes a modified nucleotide triphosphate which lacks a 3′-OH. In embodiments, the chain-terminating nucleotide includes any nucleotide triphosphate which lacks a 3′-OH. In embodiments, the blocking element includes a modified nucleotide triphosphate which lacks a 3′-OH.

In embodiments, the method further includes generating a sequencing read. In embodiments, generating a sequencing read includes executing a plurality of sequencing cycles, each cycle including extending the sequencing primer by incorporating a nucleotide or nucleotide analogue using a polymerase and detecting a characteristic signature indicating that the nucleotide or nucleotide analogue has been incorporated. In embodiments, the method further includes incorporating one or more unmodified dNTPs or one or more ddNTPs into the 3′ end of the extended sequencing primer.

In embodiments, sequencing includes (i) extending a sequencing primer by incorporating a labeled nucleotide, or labeled nucleotide analogue and (ii) detecting the label to generate a signal for each incorporated nucleotide or nucleotide analogue.

In embodiments, generating a sequencing read includes sequencing by synthesis, sequencing-by-binding, sequencing by ligation, or pyrosequencing.

In embodiments, the blocked first extension strand is downstream of the blocked second extension strand, third extension strand, or both the blocked second extension strand and third extension strand.

In embodiments, one or more of the extension strands is adjacent (e.g., having 1 or no nucleotides separating a first extension strand and a second extension strand) to one or more different extension strands. In embodiments, one or more of the extension strands is 1 nucleotide or less apart from one or more different extension strands. In embodiments, the 3′ end of one or more of the extension strands is capable of ligating to the 5′ end of one or more different extension strands (e.g., there are no nucleotides separating the 3′ end of a first extension strand and the 5′ end of a second extension strand). In embodiments, the 3′ end of the first extension strand is capable of ligating to the 5′ end of the second extension strand.

In embodiments, one or more of the extension strands is not adjacent to one or more different extension strands. In embodiments, one or more of the extension strands is between 1 to about 10 nucleotides apart from one or more different extension strands. In embodiments, one or more of the extension strands is between about 10 to about 25, about 25 to about 50, about 50 to about 75, about 75 to about 100, about 100 to about 150, or about 150 to about 300 nucleotides apart from one or more different extension strands. In embodiments, one or more of the extension strands is more than about 10, about 25, about 50, about 75, about 100, about 150, about 200, about 300, or more nucleotides apart from one or more different extension strands.

In embodiments, the method further includes contacting the template nucleic acid with a blocking element thereby terminating extension of the third extension strand thereby forming a blocked third extension strand. In embodiments, the method further includes contacting the template nucleic acid with a blocking element thereby terminating extension of the third extension strand and creating a blocked third extension strand.

In embodiments, contacting the template nucleic acid with a blocking element includes hybridizing a blocking oligonucleotide downstream of the extension strand. In embodiments, contacting the template nucleic acid with a blocking element includes hybridizing a blocking oligonucleotide downstream of the first extension strand, second extension strand, third extension strand, or fourth extension strand. In embodiments, the blocking oligonucleotide includes locked nucleic acids (LNAs), Bis-locked nucleic acids (bisLNAs), twisted intercalating nucleic acids (TINAs), bridged nucleic acids (BNAs), 2′-O-methyl RNA:DNA chimeric nucleic acids, minor groove binder (MGB) nucleic acids, morpholino nucleic acids, C5-modified pyrimidine nucleic acids, peptide nucleic acids (PNAs), phosphorothioate nucleic acids, or combinations thereof. In embodiments, the blocking oligonucleotide inhibits nucleotide incorporation. In embodiments, the blocking oligonucleotide is non-extendable.

In embodiments, the blocking element includes an oligo, a protein, or a combination thereof. In embodiments, the blocking element includes an oligo. In embodiments, the blocking element is an oligo. In embodiments, the blocking element is an oligonucleotide having 5-25 nucleotides. In embodiments, the blocking element is an oligonucleotide having 10-50 nucleotides. In embodiments, the blocking element is an oligonucleotide having 20-75 nucleotides. In embodiments, the blocking element is an oligonucleotide having about 5, about 10, about 20, about 25, about 50, or about 75 nucleotides. In embodiments, the blocking element is a non-extendable oligomer. In embodiments, the blocking element includes two or more tandemly arranged oligos. In embodiments, the blocking element is a single-stranded oligonucleotide having a 5′ end and a 3′ end. In embodiments, the blocking element includes a 3′-blocked oligo. In embodiments, the blocking element includes a blocking moiety on the 3′ nucleotide. A blocking moiety on a nucleotide can be reversible, whereby the blocking moiety can be removed or modified to allow the 3′ hydroxyl to form a covalent bond with the 5′ phosphate of another nucleotide. For example, a reversible terminator may refer to a blocking moiety located, for example, at the 3′ position of the nucleotide and may be a chemically cleavable moiety such as an allyl group, an azidomethyl group or a methoxymethyl group, or may be an enzymatically cleavable group such as a phosphate ester. In embodiments the blocking moiety is not reversible (e.g., the blocking element including a blocking moiety irreversibly prevents extension).

In embodiments, the blocking element includes an oligo having a 3′ dideoxynucleotide (ddNTP) or similar modification to prevent extension by a polymerase and is used in conjunction with a non-strand displacing polymerase. In some embodiments, the blocking oligomer contains one or more non-natural bases that facilitate hybridization of the blocker to the target sequence (e.g., LNA bases). In some embodiments, the blocking oligomer contains other modified bases to increase resistance to exonuclease digestion (e.g., one or more phosphorothioate bonds). In embodiments, the blocking element is an oligonucleotide including one or more modified nucleotides, such as iso dGTP or iso dCTP, which are complementary to each other. In a reaction of polymerization lacking the complementary modified nucleotides, extension is blocked. In another embodiment, the blocking element is an oligonucleotide including a 3′ cleavable linker containing PEG, thereby blocking extension. In another embodiment, the blocking element is an oligonucleotide including one or more sequences which are recognized and bound by one or more short RNA or PNA oligos, thereby blocking the extension by a strand displacing DNA polymerase that cannot strand displace RNA or PNA. In embodiments, the blocking element is a modified nucleotide (e.g., a nucleotide including a reversible terminator, such as a 3′-reversible terminating moiety).

As described in US2010/0167353, a number of blocking elements are known in the art that can be placed at or near the 3′ end of the oligonucleotide (e.g., a primer) to prevent extension. A primer or other oligonucleotide may be modified at the 3′-terminal nucleotide to prevent or inhibit initiation of DNA synthesis by, for example, the addition of a 3′ deoxyribonucleotide residue (e.g., cordycepin), a 2′,3′-dideoxyribonucleotide residue, non-nucleotide linkages or alkane-diol modifications (U.S. Pat. No. 5,554,516). Alkane diol modifications which can be used to inhibit or block primer extension have also been described by Wilk et al., (1990 Nucleic Acids Res. 18 (8):2065), and by Arnold et al. (U.S. Pat. No. 6,031,091). Additional examples of suitable blocking groups include 3′ hydroxyl substitutions (e.g., 3′-phosphate, 3′-triphosphate or 3′-phosphate diesters with alcohols such as 3-hydroxypropyl), 2′3′-cyclic phosphate, 2′ hydroxyl substitutions of a terminal RNA base (e.g., phosphate or sterically bulky groups such as triisopropyl silyl (TIPS) or tert-butyl dimethyl silyl (TBDMS)). 2′-alkyl silyl groups such as TIPS and TBDMS substituted at the 3′-end of an oligonucleotide are described in US2007/0218490, which is incorporated herein by reference. Bulky substituents can also be incorporated on the base of the 3′-terminal nucleotide of the oligonucleotide to block primer extension. In embodiments, the oligonucleotide may include a cleavage domain that is located upstream (e.g., 5′ to) of the blocking group used to inhibit primer extension. As examples, the cleavage domain may be an RNase H cleavage domain, or the cleavage domain may be an RNase H₂ cleavage domain comprising a single RNA residue, or the oligonucleotide may comprise replacement of the RNA base with one or more alternative nucleosides. Additional illustrative cleavage domains are described in US2010/0167353.

In embodiments, the blocking element includes an oligo, a protein, or a combination thereof. In embodiments, the blocking element includes a protein. In embodiments, the blocking element includes one or more proteins. The blocking element need not be an oligomer; in some embodiments, for example, the blocking element is a protein that selectively binds to the target sequence and prevents polymerase extension. In embodiments, the blocking element is an oligonucleotide including one or more modified nucleotides. In embodiments, the blocking element is an oligonucleotide including one or more modified nucleotides, wherein one or more modified nucleotides is linked to biotin, to which a protein (e.g., streptavidin) can be bound, thereby blocking polymerase extension. In embodiments, the blocking element includes one or more sequences which is recognized and bound by one or more single-stranded DNA-binding proteins, thereby blocking polymerase extension at the bound site.

In embodiments, the sequence that specifically binds the blocking element is about 1 to about 100 nucleotides from the extension strand (e.g., the upstream extension strand, or extended strand located 5′ to the sequence that specifically binds the blocking element). In embodiments, the sequence that specifically binds the blocking element is about 5 to about 100 nucleotides from the extension strand. In embodiments, the sequence that specifically binds the blocking element is about 10 to about 100 nucleotides from the extension strand. In embodiments, the sequence that specifically binds the blocking element is about 25 to about 100 nucleotides from the extension strand. In embodiments, the sequence specifically binds the blocking element is about 50 to about 100 nucleotides from the extension strand. In embodiments, the sequence that specifically binds the blocking element is about 75 to about 100 nucleotides from the extension strand. In embodiments, the sequence that specifically binds the blocking element is about 1, about 5, about 10, about 25, about 50, about 75, or about 100 nucleotides from the extension strand. In embodiments, the sequence that specifically hybridizes to the blocking element does not overlap with the extension strand. In embodiments, the sequence that specifically hybridizes to the extension strand and the sequence that specifically hybridizes to the blocking elements are about 5, about 10, or about 20 nucleotides apart.

In embodiments, the method further includes repeating steps (a)-(b), thereby sequencing an additional region of the template nucleic acid. In embodiments, the method further includes repeating steps (a)-(b) one or more times, thereby sequencing one or more additional regions of the template nucleic acid. In embodiments, the method further includes contacting a fourth primer annealed to a fourth region of the template nucleic acid and incorporating one or more nucleotides into the fourth primer with a polymerase to create a fourth extension strand, and detecting the one or more incorporated nucleotides so as to identify each incorporated nucleotide in the fourth extension strand.

In embodiments, between 4 to 9 regions or 9 to 15 regions of the template nucleic acid are sequenced. In embodiments, 4, 5, 6, 7, 8, 9, 10, 11, 12, 13, 14, or 15 regions of the template nucleic acid are sequenced. In embodiments, between 15 to 30 regions or 30 to 50 regions of the template nucleic acid are sequenced. In embodiments, 15, 16, 17, 18, 19, 20, 21, 22, 23, 24, 25, 26, 27, 28, 29, or 30 regions of the template nucleic acid are sequenced. In embodiments, about 30, about 35, about 40, about 45, about 50, or more regions of the template nucleic acid are sequenced.

In embodiments, the sequenced region of the template nucleic acid is between about 5 to about 200 nucleotides in length. In embodiments, the sequenced region of the template nucleic acid is between about 10 to about 25 nucleotides in length. In embodiments, the sequenced region of the template nucleic acid is between about 25 to about 50 nucleotides in length. In embodiments, the sequenced region of the template nucleic acid is between about 50 to about 75 nucleotides in length. In embodiments, the sequenced region of the template nucleic acid is between about 75 to about 100 nucleotides in length. In embodiments, the sequenced region of the template nucleic acid is between about 100 to about 125 nucleotides in length. In embodiments, the sequenced region of the template nucleic acid is between about 125 to about 150 nucleotides in length. In embodiments, the sequenced region of the template nucleic acid is between about 150 to about 175 nucleotides in length. In embodiments, the sequenced region of the template nucleic acid is between about 175 to about 200 nucleotides in length. In embodiments, the sequenced region of the template nucleic acid is more than about 200 nucleotides in length. In embodiments, the sequenced region of the template nucleic acid is about 5, 10, 15, 20, 25, 50, 75, 100, 125, 150, 175, 200, or more nucleotides in length.

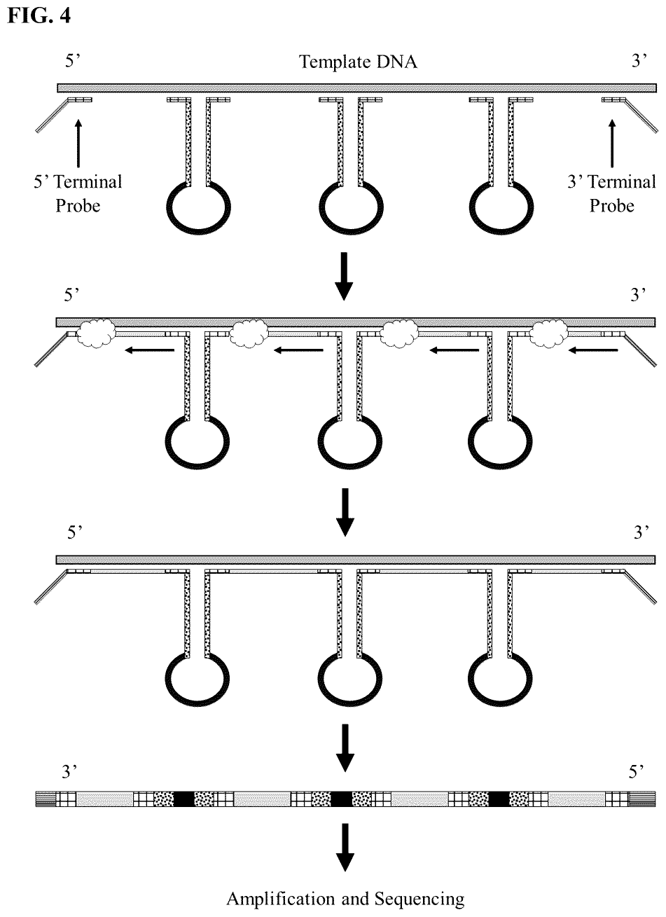

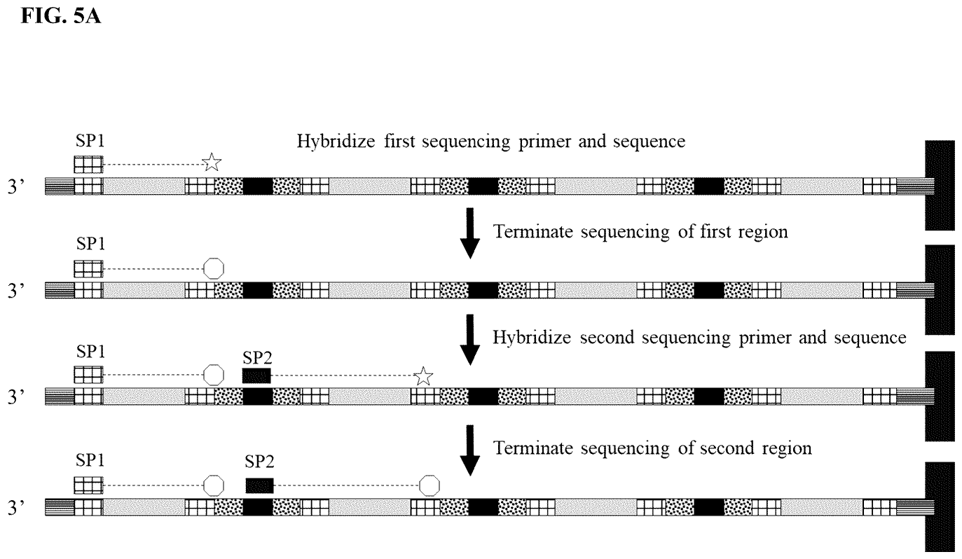

In an aspect is provided method of forming an integrated strand complement of a template nucleic acid including a plurality of oligonucleotide probes, the method including: a. hybridizing one or more interposing oligonucleotide probes to the template nucleic acid, hybridizing a 5′ terminal oligonucleotide probe downstream of the one or more interposing oligonucleotide probes, and hybridizing a 3′ terminal oligonucleotide probe upstream of the one or more interposing oligonucleotide probes (see, e.g., FIG. 4 for an illustration of a template nucleic acid including a plurality of oligonucleotide probes), wherein each of the interposing oligonucleotide probes includes from 5′ to 3′: i. a first hybridization sequence complementary to a first sequence of the template nucleic acid; ii. a loop region including a primer binding sequence; and iii. a second hybridization sequence complementary to a second sequence of the template nucleic acid; wherein the 5′ terminal oligonucleotide probe includes from 5′ to 3′: i. a hybridization sequence complementary to a third sequence of the template nucleic acid; and ii. a primer binding sequence; and wherein the 3′ terminal oligonucleotide probe includes from 3′ to 5′: i. a hybridization sequence complementary to a fourth sequence of the template nucleic acid; and ii. a primer binding sequence; b. extending the 3′ end of each second hybridization sequence of the interposing oligonucleotide probes and the 3′ end of the hybridization sequence of the 3′ terminal oligonucleotide probe with one or more polymerases thereby forming an extension product of each of the oligonucleotide probes; c. ligating the 3′ end of each of the extension products to the 5′ end of the adjacent extension products, and ligating the 5′ end of the 5′ terminal oligonucleotide probe to the 3′ end of the adjacent extension product, each hybridized to the same template nucleic acid thereby making an integrated strand including a complement of the template nucleic acid including a plurality of the oligonucleotide probes; and d. amplifying the integrated strand by an amplification reaction to produce a complement of the integrated strand thereby forming an integrated strand complement of the template nucleic acid including oligonucleotide probes, wherein the complement of the integrated strand includes a complement of the plurality of oligonucleotide probes.

In embodiments, the 5′ terminal oligonucleotide probe includes from 5′ to 3′: i. a hybridization sequence complementary to a 5′ terminal sequence of the template nucleic acid, wherein the 5′ terminal sequence is downstream of the template nucleic acid sequence complementary to the interposing oligonucleotide probes; and ii. a primer binding sequence; and the 3′ terminal oligonucleotide probe includes from 3′ to 5′: i. a hybridization sequence complementary to a 3′ terminal sequence of the template nucleic acid, wherein the 3′ terminal sequence is upstream of the template nucleic acid sequence complementary to the interposing oligonucleotide probes (see, e.g., FIG. 4 for an illustration of the hybridized 5′ and 3′ terminal oligonucleotide probes); and ii. a primer binding sequence.

In embodiments, amplifying the integrated strand by an amplification reaction generates a plurality of amplification products (e.g., a plurality of integrated strand complements). In embodiments, the amplification reaction includes contacting the integrated strand with an amplification primer. In embodiments, amplifying includes hybridizing an amplification primer to the integrated strands and cycles of primer extension with a polymerase and nucleotides to generate amplified products. In embodiments, the amplification reaction includes polymerase chain reaction (PCR), strand displacement amplification (SDA), multiple displacement amplification (MDA), ligation chain reaction, transcription mediated amplification (TMA), nucleic acid sequence based amplification (NASBA), rolling circle amplification (RCA), exponential rolling circle amplification (eRCA), hyperbranched rolling circle amplification (HRCA), or a combination thereof.

In embodiments, the 5′ terminal oligonucleotide probe includes from 5′ to 3′: i. a first hybridization sequence complementary to a first 5′ terminal sequence of the template nucleic acid; ii. a loop region including a primer binding sequence; and iii. a second hybridization sequence complementary to a second 5′ terminal sequence of the template nucleic acid, wherein the first and second 5′ terminal sequences are upstream of the template nucleic acid sequence complementary to the interposing oligonucleotide probes.

In embodiments, the 3′ terminal oligonucleotide probe includes from 3′ to 5′: i. a first hybridization sequence complementary to a first 3′ terminal sequence of the template nucleic acid; ii. a loop region including a primer binding sequence; and iii. a second hybridization sequence complementary to a second 3′ terminal sequence of the template nucleic acid, wherein the first and second 3′ terminal sequences are downstream of the template nucleic acid sequence complementary to the interposing oligonucleotide probes.

In embodiments, the first hybridization sequence, the second hybridization sequence, and the primer binding sequence (e.g., the primer binding sequence of the loop region) is different between each of the plurality of interposing oligonucleotide probes. In embodiments, the first hybridization sequence, the second hybridization sequence, and/or the primer binding sequence is different between the 5′ terminal oligonucleotide probe and the 3′ terminal oligonucleotide probe.

In an aspect is provided a method of sequencing a plurality of different regions (e.g., 3 different regions) of a template nucleic acid, the method including: forming a detection complex including a primed complex and a reversibly terminated nucleotide, wherein the primed complex includes a polymerase and a primer annealed to a first region of the nucleic acid template; detecting the detection complex; with a polymerase, forming a complex including the primer, the nucleic acid template, a nucleotide; and binding and detecting the nucleotides.

In embodiments, the template nucleic acid includes a gene or a gene fragment. In embodiments, the gene or gene fragment is a cancer-associated gene or fragment thereof, T cell receptor (TCRs) gene or fragment thereof, or a B cell receptor (BCRs) gene, or fragment thereof. In embodiments, the gene or gene fragment is a CDR3 gene or fragment thereof. In embodiments, the gene or gene fragment is a T cell receptor alpha variable (TRAV) gene or fragment thereof, T cell receptor alpha joining (TRAJ) gene or fragment thereof, T cell receptor alpha constant (TRAC) gene or fragment thereof, T cell receptor beta variable (TRBV) gene or fragment thereof, T cell receptor beta diversity (TRBD) gene or fragment thereof, T cell receptor beta joining (TRBJ) gene or fragment thereof, T cell receptor beta constant (TRBC) gene or fragment thereof, T cell receptor gamma variable (TRGV) gene or fragment thereof, T cell receptor gamma joining (TRGJ) gene or fragment thereof, T cell receptor gamma constant (TRGC) gene or fragment thereof, T cell receptor delta variable (TRDV) gene or fragment thereof, T cell receptor delta diversity (TRDD) gene or fragment thereof, T cell receptor delta joining (TRDJ) gene or fragment thereof, or T cell receptor delta constant (TRDC) gene or fragment thereof. In embodiments, the template nucleic acid is DNA. In embodiments, the polynucleotide is RNA. In embodiments, the template is a double stranded polynucleotide. In embodiments, the template nucleic acid is a single stranded polynucleotide. In embodiments, the polynucleotide includes genomic DNA, complementary DNA (cDNA), cell-free DNA (cfDNA), messenger RNA (mRNA), transfer RNA (tRNA), ribosomal RNA (rRNA), cell-free RNA (cfRNA), or noncoding RNA (ncRNA). In embodiments, the polynucleotide includes messenger RNA (mRNA), transfer RNA (tRNA), micro-RNA (miRNA), small interfering RNA (siRNA), small nucleolar RNA (snoRNA), small nuclear RNA (snRNA), Piwi-interacting RNA (piRNA), enhancer RNA (eRNA), or ribosomal RNA (rRNA).

In embodiments, the integrated strand complement is at least 1000 bases (1 kb), at least 2 kb, at least 4 kb, at least 6 kb, at least 10 kb, at least 20 kb, at least 30 kb, at least 40 kb, or at least 50 kb in length. In embodiments, the entire sequence of the integrated strand complement is about 1 to 3 kb, and only a portion of that the integrated strand complement (e.g., 50 to 100 nucleotides) is sequenced at a time. In embodiments, the integrated strand complement is about 2 to 3 kb. In embodiments, the integrated strand complement is about 1 to 10 kb. In embodiments, the integrated strand complement is about 3 to 10 kb. In embodiments, the integrated strand complement is about 5 to 10 kb. In embodiments, the integrated strand complement is about 1 to 3 kb. In embodiments, the integrated strand complement is about 1 to 2 kb. In embodiments, the integrated strand complement is greater than 1 kb. In embodiments, the integrated strand complement is greater than 500 bases. In embodiments, the integrated strand complement is about 1 kb. In embodiments, the integrated strand complement is about 2 kb. In embodiments, the integrated strand complement is less than 1 kb. In embodiments, the integrated strand complement is about 500 nucleotides. In embodiments, the integrated strand complement is about 510 nucleotides. In embodiments, the integrated strand complement is about 520 nucleotides. In embodiments, the integrated strand complement is about 530 nucleotides. In embodiments, the integrated strand complement is about 540 nucleotides. In embodiments, the integrated strand complement is about 550 nucleotides. In embodiments, the integrated strand complement is about 560 nucleotides. In embodiments, the integrated strand complement is about 570 nucleotides. In embodiments, the integrated strand complement is about 580 nucleotides. In embodiments, the integrated strand complement is about 590 nucleotides. In embodiments, the integrated strand complement is about 600 nucleotides. In embodiments, the integrated strand complement is about 610 nucleotides. In embodiments, the integrated strand complement is about 620 nucleotides. In embodiments, the integrated strand complement is about 630 nucleotides. In embodiments, the integrated strand complement is about 640 nucleotides. In embodiments, the integrated strand complement is about 650 nucleotides. In embodiments, the integrated strand complement is about 660 nucleotides. In embodiments, the integrated strand complement is about 670 nucleotides. In embodiments, the integrated strand complement is about 680 nucleotides. In embodiments, the integrated strand complement is about 690 nucleotides. In embodiments, the integrated strand complement is about 700 nucleotides. In embodiments, the integrated strand complement is about 1,600 nucleotides. In embodiments, the integrated strand complement is about 1,610 nucleotides. In embodiments, the integrated strand complement is about 1,620 nucleotides. In embodiments, the integrated strand complement is about 1,630 nucleotides. In embodiments, the integrated strand complement is about 1,640 nucleotides. In embodiments, the integrated strand complement is about 1,650 nucleotides. In embodiments, the integrated strand complement is about 1,660 nucleotides. In embodiments, the integrated strand complement is about 1,670 nucleotides. In embodiments, the integrated strand complement is about 1,680 nucleotides. In embodiments, the integrated strand complement is about 1,690 nucleotides. In embodiments, the integrated strand complement is about 1,700 nucleotides. In embodiments, the integrated strand complement is about 1,710 nucleotides. In embodiments, the integrated strand complement is about 1,720 nucleotides. In embodiments, the integrated strand complement is about 1,730 nucleotides. In embodiments, the integrated strand complement is about 1,740 nucleotides. In embodiments, the integrated strand complement is about 1,750 nucleotides. In embodiments, the integrated strand complement is about 1,760 nucleotides. In embodiments, the integrated strand complement is about 1,770 nucleotides. In embodiments, the integrated strand complement is about 1,780 nucleotides. In embodiments, the integrated strand complement is about 1,790 nucleotides. In embodiments, the integrated strand complement is about 1,800 nucleotides.

In embodiments, prior to step (a), the template nucleic acid is immobilized to a solid support, wherein the solid support includes a plurality of immobilized primers. In embodiments, prior to step (a), the template nucleic acid is contacted to a first immobilized primer on the solid support and the primer is extended by a polymerase, thereby generating an immobilized complement template nucleic acid. In embodiments, the template nucleic acid is removed with a denaturant and the immobilized complement template nucleic acid is contacted with a second immobilized primer. In embodiments, the second immobilized primer is extended with a polymerase, thereby generating an immobilized template nucleic acid.

In embodiments, the interposing oligonucleotide probes (alternatively referred to herein as interposing probes (IPPs)) provided herein include a first and second hybridization sequence that are complementary to a first and second sequence of a template nucleic acid, respectively. In embodiments, each hybridization sequence includes about 10 to about 25 nucleotides. In embodiments, each hybridization sequence includes about 3 to about 5 nucleotides. In embodiments, each hybridization sequence has 3 to 5 nucleotides. In embodiments, the first hybridization sequence includes more nucleotides than the second hybridization sequence. In embodiments, the first hybridization sequence includes about 3 to about 5 nucleotides and the second hybridization sequence includes about 3 to 25 nucleotides. In embodiments, the first hybridization sequence includes about 3 to about 25 nucleotides and the second hybridization sequence includes about 3 to 5 nucleotides. In embodiments, the first hybridization sequence includes about 3 to about 25 nucleotides and the second hybridization sequence includes about 3 to 25 nucleotides. In embodiments, the first hybridization sequence includes about 10 to about 25 nucleotides and the second hybridization sequence includes about 10 to 5 nucleotides. In embodiments, the first hybridization sequence includes about 10 to about 15 nucleotides and the second hybridization sequence includes about 10 to 15 nucleotides. In embodiments, the interposing oligonucleotide probes provided herein include a hybridization sequence that includes about 1 to about 20 nucleotides, about 5 to about 15 nucleotides, or about 8 to about 12 nucleotides. In embodiments, the interposing oligonucleotide probes include a hybridization sequence that includes about 9 to about 18 nucleotides. In embodiments, the interposing oligonucleotide probes include a hybridization sequence that includes a targeted primer sequence, i.e., a nucleic acid sequence that is complementary to a known nucleic acid region. For example, the targeted primer sequence may be complementary to a universally conserved region, or complementary sequences to target specific genes or mutations that have relevancy to a particular cancer phenotype. In embodiments, the total combined length of the first hybridization sequence and the second hybridization sequence includes about 18 to about 25 nucleotides. In embodiments, the total combined length of the first hybridization sequence and the second hybridization sequence includes about 25 to about 50 nucleotides.

In embodiments, the interposing oligonucleotide probes provided herein include a hybridization sequence that includes about 1 to about 10 nucleotides, about 2 to about 9 nucleotides, about 3 to about 8 nucleotides, about 4 to about 7 nucleotides, or about 5 to about 6 nucleotides. In embodiments, the interposing oligonucleotide probes provided herein include a hybridization sequence that includes 1, 2, 3, 4, 5, 6, 7, 8, 9, or 10 nucleotides. In embodiments, the interposing oligonucleotide probes provided herein include a hybridization sequence that includes 3 nucleotides. In embodiments, the interposing oligonucleotide probes provided herein include a hybridization sequence that includes 4 nucleotides. In embodiments, the interposing oligonucleotide probes provided herein include a hybridization sequence that includes 5 nucleotides. In embodiments, the interposing oligonucleotide probes provided herein include a hybridization sequence that includes 6 nucleotides. In embodiments, the interposing oligonucleotide probes provided herein include a hybridization sequence that includes 7 nucleotides. In embodiments, the interposing oligonucleotide probes provided herein include a hybridization sequence that includes 8 nucleotides. In embodiments, the interposing oligonucleotide probes provided herein include two hybridization sequences, and each hybridization sequence consists of 4 nucleotides. In embodiments, the interposing oligonucleotide probes provided herein include two hybridization sequences, and each hybridization sequence consists of 5 nucleotides. In embodiments, the interposing oligonucleotide probes provided herein include two hybridization sequences, and each hybridization sequence consists of 6 nucleotides. In embodiments, the interposing oligonucleotide probes provided herein include two hybridization sequences, and each hybridization sequence consists of 7 nucleotides. In embodiments, the interposing oligonucleotide probes provided herein include two hybridization sequences, and each hybridization sequence consists of 8 nucleotides. In embodiments, the interposing oligonucleotide probes provided herein include two hybridization sequences, and each hybridization sequence consists of 9 nucleotides. In embodiments, the interposing oligonucleotide probes provided herein include two hybridization sequences, and each hybridization sequence consists of 10 nucleotides. In embodiments, the interposing oligonucleotide probes provided herein include two hybridization sequences, and each hybridization sequence consists of 11 nucleotides. In embodiments, the interposing oligonucleotide probes provided herein include two hybridization sequences, and each hybridization sequence consists of 12 nucleotides. In embodiments, the interposing oligonucleotide probes include a hybridization sequence having a first sequence (e.g., ATTG) and a second sequence (e.g., CCTA) that are independently different from each other. In embodiments, the interposing oligonucleotide probes include a hybridization sequence having a first sequence (e.g., TACG) and a second sequence (e.g., TACG) that are identical. In embodiments, the interposing oligonucleotide probes include a hybridization sequence having a first sequence (e.g., ATTG) and a second sequence (e.g., CCTATTACGATAACA (SEQ ID NO:2)) that are independently different from each other. In embodiments, the first hybridization sequence includes a targeted primer sequence, or a portion thereof. In embodiments, the second hybridization sequence includes a targeted priming sequence, or a portion thereof.

In embodiments, the hybridization sequence includes at least one target-specific region (also referred to herein as a target priming sequence). A target-specific region is a single stranded polynucleotide that is at least 50% complementary, at least 75% complementary, at least 85% complementary, at least 90% complementary, at least 95% complementary, at least 98%, at least 99% complementary, or 100% complementary to a portion of a nucleic acid molecule that includes a known target sequence (e.g., a gene or gene fragment of interest). In embodiments, the target-specific region is capable of hybridizing to at least a portion of the target sequence. In embodiments, the target-specific region is substantially non-complementary to other target sequences present in the sample.

The melting temperature (Tm) of an interposing probe can be changed (e.g., increased) to a desired Tm using a suitable method, for example by changing (e.g., increasing) GC content, changing (e.g., increasing) length and/or by the inclusion of modified nucleotides, nucleotide analogues and/or modified nucleotides bonds, non-limiting examples of which include locked nucleic acids (LNAs, e.g., bicyclic nucleic acids), bridged nucleic acids (BNAs, e.g., constrained nucleic acids), C5-modified pyrimidine bases (for example, 5-methyl-dC, propynyl pyrimidines, among others) and alternate backbone chemistries, for example peptide nucleic acids (PNAs), morpholinos, the like or combinations thereof. In embodiments, the interposing probes include nucleotide analogues to increase binding stability (e.g., Locked Nucleic Acid bases (LNAs)). For example, an interposing probe that includes synthetic analogue bases such as LNAs (e.g., LNAs as described in US 2003/0092905; U.S. Pat. No. 7,084,125, which are incorporated herein by reference for all purposes) may increase the Tm. In embodiments, the interposing probe includes a plurality of LNAs (e.g., 2, 3, 4, 5, 6, 7, 8, 9, or 10 LNAs). In embodiments, the interposing probe includes 2-6 LNAs. In embodiments, the hybridization sequence includes one or more modified nucleotides, such as LNAs. In embodiments, each hybridization sequence includes one or more LNAs. In embodiments, the interposing probe has the general formula 5′-[hybridization sequence 1 domain]-[stem 1 domain]-[loop domain]-[stem 2 domain]-[hybridization sequence 2 domain]-3′. In embodiments, the interposing probe has the general formula 5′-[hybridization sequence 1 domain]-[loop domain]-[hybridization sequence 2 domain]-3′. In embodiments, the interposing probe has the formula: 5′Phos-[hybridization sequence 1 domain]-[stem 1 domain]-[loop domain]-[stem 2 domain]-[hybridization sequence 2 domain]-3′, wherein the hybridization sequence 1 domain has the sequence: ACCACG+GTCAC (SEQ ID NO:3); stem 1 domain has the sequence: CTCCAC (SEQ ID NO:4); stem 2 domain has the sequence: GTGGAG (SEQ ID NO: 5); and the hybridization sequence 2 domain has the sequence CGT+CTCCTCAG (SEQ ID NO:6), wherein +G and +C represent the LNA bases. In embodiments, the interposing probe has the formula: 5′Phos-[hybridization sequence 1 domain]-[loop domain]-[hybridization sequence 2 domain]-3′, wherein the hybridization sequence 1 domain has the sequence: ACCACG+GTCAC (SEQ ID NO:3); and the hybridization sequence 2 domain has the sequence CGT+CTCCTCAG (SEQ ID NO:6), wherein +G and +C represent the LNA bases. In embodiments, the loop domain does not contain random nucleotides. In embodiments, the loop domain includes one or more primer binding sequences. In embodiments, the Tm of hybridization sequence is greater than 40° C. In embodiments, the Tm of hybridization sequence is greater than 45° C.

In embodiments, the interposing oligonucleotide probes provided herein include a first and second hybridization sequence that include targeting priming sequences, or a portion thereof. In embodiments, the interposing oligonucleotide probes provided herein do not include a first and second hybridization sequence that include randomly generated sequences.

In embodiments, the interposing oligonucleotide probes provided herein include a first and second stem region. In embodiments, the interposing oligonucleotide probes provided herein do not include a first and second stem region. The first and second stem regions are composed of complementary nucleotide sequences. In embodiments, the first stem region includes a sequence common to a plurality of the interposing oligonucleotide probes. In embodiments, the second stem region includes a sequence complementary to the first stem region, where the second stem region is capable of hybridizing to the first stem region under hybridization conditions.

In embodiments, the interposing oligonucleotide probes include a loop region that includes a primer binding sequence, which optionally may function as a molecular identifier when assembled and aligned using bioinformatic algorithms known in the art and described herein. In embodiments, the loop region alone may be considered a molecular identifier. In embodiments, the loop region further includes a sample index sequence. In embodiments, the interposing oligonucleotide probes do not include a UMI.

In embodiments, the first and second stem regions of the interposing oligonucleotide probes provided herein include a known sequence of about 5 to about 10 nucleotides. In embodiments, the first and second stem regions of the interposing oligonucleotide probes provided herein include a known sequence of about 1 to about 20 nucleotides, about 2 to about 19, about 3 to about 18 nucleotides, about 4 to about 17 nucleotides, about 5 to about 16 nucleotides, about 6 to about 15 nucleotides, about 7 to about 14 nucleotides, about 8 to about 13 nucleotides, about 9 to about 12 nucleotides, or about 10 to about 11 nucleotides. In embodiments, the first and second stem regions of the interposing oligonucleotide probes provided herein include a known sequence of about 1, 2, 3, 4, 5, 6, 7, 8, 9, 10, 11, 12, 13, 14, 15, 16, 17, 18, 19, or about 20 nucleotides. In embodiments of the interposing oligonucleotide probes provided herein, the first stem region includes about 5 nucleotides. In embodiments of the interposing oligonucleotide probes provided herein, the first stem region includes about 6 nucleotides. In embodiments of the interposing oligonucleotide probes provided herein, the first stem region includes about 7 nucleotides. In embodiments of the interposing oligonucleotide probes provided herein, the first stem region includes about 8 nucleotides. In embodiments of the interposing oligonucleotide probes provided herein, the first stem region includes about 9 nucleotides. In embodiments of the interposing oligonucleotide probes provided herein, the first stem region includes about 10 nucleotides. In embodiments of the interposing oligonucleotide probes provided herein, the second stem region includes about 5 nucleotides. In embodiments of the interposing oligonucleotide probes provided herein, the second stem region includes about 6 nucleotides. In embodiments of the interposing oligonucleotide probes provided herein, the second stem region includes about 7 nucleotides. In embodiments of the interposing oligonucleotide probes provided herein, the second stem region includes about 8 nucleotides. In embodiments of the interposing oligonucleotide probes provided herein, the second stem region includes about 9 nucleotides. In embodiments of the interposing oligonucleotide probes provided herein, the second stem region includes about 10 nucleotides. In embodiments, the first and second stem regions are substantially complementary to each other.

In embodiments, the interposing oligonucleotide probes provided herein include a loop region that further includes a sample index sequence. In general, a sample index sequence is the same for all polynucleotides from the same sample source (e.g., the same subject, the same aliquot, or the same container), and differs from the sample index sequence of polynucleotides from a different sample source. Polynucleotides from different samples can therefore be mixed, and the sequences subsequently grouped by sample source by virtue of the sample index sequence. In embodiments, the sample index sequence is a randomly generated sequence that is sufficiently different from other sample index sequences to allow the identification of the sample source based on index sequence(s) with which they are associated. In embodiments, each sample index sequence in a plurality of index sequences differs from every other index sequence in the plurality by at least three nucleotide positions, such as at least 3, 4, 5, 6, 7, 8, 9, 10, or more nucleotide positions. In some embodiments, substantially degenerate index sequences may be known as random. In some embodiments a sample index sequence may include a nucleic acid sequence from within a pool of known sequences. In some embodiments, the sample index sequences may be pre-defined. In embodiments, the sample index sequence includes about 1 to about 10 nucleotides. In embodiments, the sample index sequence includes about 3, 4, 5, 6, 7, 8, 9, or about 10 nucleotides. In embodiments, the sample index sequence includes about 3 nucleotides. In embodiments, the sample index sequence includes about 5 nucleotides. In embodiments, the sample index sequence includes about 7 nucleotides. In embodiments, the sample index sequence includes about 10 nucleotides. In embodiments, the sample index sequence includes about 11 nucleotides. In embodiments, the sample index sequence includes about 12 nucleotides. In embodiments, the sample index sequence includes about 8 to 15 nucleotides. In embodiments, the sample index sequence includes 12 nucleotides.

In embodiments, the interposing oligonucleotide probes provided herein include a loop region. In embodiments, the loop region, alone or in combination with a sequence of one or both of (a) the template nucleic acid, or (b) one or more probe sequences, uniquely distinguishes the template nucleic acid from other template nucleic acids in a plurality of template nucleic acids. In embodiments of the interposing oligonucleotide probes provided herein, the loop region includes about 5 to about 20 nucleotides or about 10 to about 20 nucleotides. In embodiments of the interposing oligonucleotide probes provided herein, the loop region includes about 25 to about 35 nucleotides or about 30 to about 50 nucleotides. In embodiments of the interposing oligonucleotide probes provided herein, the loop region includes about 1 to about 25, about 2 to about 24, about 3 to about 23, about 4 to about 22, about 5 to about 21, about 6 to about 20, about 7 to about 19, about 8 to about 18, about 9 to about 17, about 10 to about 16, about 11 to about 15, or about 12 to about 14 nucleotides. In embodiments of the interposing oligonucleotide probes provided herein, the loop region includes about 1, 2, 3, 4, 5, 6, 7, 8, 9, 10, 11, 12, 13, 14, 15, 16, 17, 18, 19, 20, 21, 22, 23, 24, or about 25 nucleotides. In embodiments of the interposing oligonucleotide probes provided herein, the loop region includes about 5 nucleotides. In embodiments of the interposing oligonucleotide probes provided herein, the loop region includes about 10 nucleotides. In embodiments of the interposing oligonucleotide probes provided herein, the loop region includes about 15 nucleotides. In embodiments of the interposing oligonucleotide probes provided herein, the loop region includes about 20 nucleotides. In embodiments, the loop region does not include a sample index sequence. In embodiments, the loop includes a TT-[UMI sequence]-TT sequence, such as TT-[NNNNNNNNNNNN]-TT (SEQ ID NO:1) sequence, wherein N represents random nucleotides and A, T, C, G represent fixed nucleotides). In embodiments, the loop includes a TT-[SP sequence]-TT sequence, wherein SP sequence represents a sequence complementary to a sequencing primer, such as SP1. In embodiments, the loop includes, from 5′ to 3′, the general formula: TT-[SP sequence]-[UMI sequence]-TT sequence, such as TT-[SP sequence]-[NNNNNNNNNNNN]-TT, wherein N represents random nucleotides and A, T, C, G represent fixed nucleotides, and wherein SP sequence represents a sequence complementary to a sequencing primer. Exemplary sequencing primers are known in the art (e.g., Nextera™ Transposase Adapters), and in embodiments, the complement of the Read 1 and Read 2 Nextera™ Transposase Adapters could be included as the SP sequence within the loop region as described herein.

In embodiments, the interposing oligonucleotide probes provided herein include a loop region that includes a probe sequence, wherein the probe sequence is selected from a set of probe sequences represented by a random or partially random sequence. In embodiments, the interposing oligonucleotide probes provided herein include a loop region that includes a probe sequence, where the probe sequence is selected from a set of probe sequences represented by a random sequence. In embodiments, the interposing oligonucleotide probes provided herein include a loop region that includes a probe sequence, where each probe sequence is selected from a set of probe sequences represented by a partially random sequence.

In embodiments, the interposing oligonucleotide probes provided herein includes a random sequence. In embodiments, the interposing oligonucleotide probes provided herein include a probe sequence that includes a random sequence. In embodiments, the random sequence excludes a subset of sequences, where the excluded subset includes sequences with three or more identical consecutive nucleotides. In embodiments, the excluded subset includes sequences with three identical consecutive nucleotides. In embodiments, the excluded subset includes sequences with four identical consecutive nucleotides. In embodiments, the excluded subset includes sequences with five identical consecutive nucleotides.

In embodiments, the interposing oligonucleotide probes provided herein include a barcode sequence, where each barcode sequence differs from every other barcode sequence by at least two nucleotide positions. In embodiments, the interposing oligonucleotide probes provided herein include barcode sequences, where each barcode sequence differs from every other barcode sequence by at least three nucleotide positions. In embodiments, the interposing oligonucleotide probes provided herein include barcode sequences, where each barcode sequence differs from every other barcode sequence by at least four nucleotide positions. In embodiments, the interposing oligonucleotide probes provided herein include barcode sequences, where each barcode sequence differs from every other barcode sequence by at least five nucleotide positions.

In embodiments, the interposing oligonucleotide probes provided herein include a loop region that includes a barcode sequence that alone or in combination with a sequence of one or both of (a) the template nucleic acid, or (b) one or more additional barcode sequences, uniquely distinguishes the template nucleic acid from other template nucleic acids in a plurality of template nucleic acids. In embodiments, the interposing oligonucleotide probes provided herein include a loop region that includes a barcode sequence that alone uniquely distinguishes the sample polynucleotide from other sample polynucleotides in a plurality of sample polynucleotides. In embodiments, the interposing oligonucleotide probes provided herein include a loop region that includes a barcode sequence that in combination with a sequence of the sample polynucleotide uniquely distinguishes the sample polynucleotide from other sample polynucleotides in a plurality of sample polynucleotides. In embodiments, the interposing oligonucleotide probes provided herein include a loop region that includes a barcode sequence that in combination with a sequence of one or more additional probe sequences, uniquely distinguishes the sample polynucleotide from other sample polynucleotides in a plurality of sample polynucleotides. In embodiments, the interposing oligonucleotide probes provided herein include a loop region that includes a barcode sequence that in combination with a sequence of the sample polynucleotide, and one or more additional probe sequences, uniquely distinguishes the sample polynucleotide from other sample polynucleotides in a plurality of sample polynucleotides.

In embodiments, the interposing oligonucleotide probes provided herein include a loop region that includes a primer binding sequence that alone or in combination with a sequence of one or both of (a) the template nucleic acid, or (b) one or more additional primer binding sequences, uniquely distinguishes the template nucleic acid from other template nucleic acids in a plurality of template nucleic acids. In embodiments, the interposing oligonucleotide probes provided herein include a loop region that includes a primer binding sequence that alone uniquely distinguishes the sample polynucleotide from other sample polynucleotides in a plurality of sample polynucleotides. In embodiments, the interposing oligonucleotide probes provided herein include a loop region that includes a primer binding sequence that in combination with a sequence of the sample polynucleotide uniquely distinguishes the sample polynucleotide from other sample polynucleotides in a plurality of sample polynucleotides. In embodiments, the interposing oligonucleotide probes provided herein include a loop region that includes a primer binding sequence that in combination with a sequence of one or more additional primer binding sequences, uniquely distinguishes the sample polynucleotide from other sample polynucleotides in a plurality of sample polynucleotides. In embodiments, the interposing oligonucleotide probes provided herein include a loop region that includes a primer binding sequence that in combination with a sequence of the sample polynucleotide, and one or more additional primer binding sequences, uniquely distinguishes the sample polynucleotide from other sample polynucleotides in a plurality of sample polynucleotides.

In embodiments, the interposing oligonucleotide probes provided herein include a 5′ phosphate moiety. A phosphate moiety attached to the 5′-end permits ligation of two nucleotides, i.e., the covalent binding of a 5′-phosphate to the 3′-hydroxyl group of another nucleotide, to form a phosphodiester bond. Removal of the 5′-phosphate prevents ligation.

In embodiments, the 5′ terminal oligonucleotide probe and/or 3′ terminal oligonucleotide probe (also referred to herein as “terminal probes” or “flanking adapters”) include one or more phosphorothioate containing nucleotides. For example, one terminal probe may include five terminal phosphorothioate linkages on the 3′ end to prevent exonuclease degradation (e.g., exonuclease degradation by T4 DNA Polymerase). In embodiments, the terminal probe includes one or more LNAs. In embodiments, the terminal probe includes a modified nucleotide that contains an affinity tag (e.g., a biotin-containing nucleotide). The biotin-containing terminal adapter, for example, could then facilitate affinity purification of the integrated strand complement of the template nucleic acid.

In embodiments, the methods of making integrated strand complements of a plurality of template nucleic acids include extending the 3′ ends of the interposing oligonucleotide probes with one or more polymerases to create extension products. Methods of extending 3′ ends of oligonucleotides are known to those skilled in the art. In embodiments, extension is achieved by a DNA polymerase without strand displacement activity.

In embodiments, the methods of making integrated strand complements of a plurality of template nucleic acids include ligating adjacent ends of extension products hybridized to the same template nucleic acid thereby making complements of the plurality of template nucleic acids integrated with a plurality of interposing oligonucleotide probes. Methods of ligation are known to those skilled in the art. In embodiments, the ligation includes enzymatic ligation. In embodiments, ligating includes enzymatic ligation including a ligation enzyme (e.g., CircLigase™ enzyme, Taq DNA Ligase, HiFi Taq DNA Ligase, T4 ligase, PBCV-1 DNA Ligase (also known as SplintR ligase) or Ampligase DNA Ligase). Non-limiting examples of ligases include DNA ligases such as DNA Ligase I, DNA Ligase II, DNA Ligase III, DNA Ligase IV, T4 DNA ligase, T7 DNA ligase, T3 DNA Ligase, E. coli DNA Ligase, PBCV-1 DNA Ligase (also known as SplintR ligase) or a Taq DNA Ligase. In embodiments, the ligating enzyme is T4 RNA ligase, T4 DNA ligase, T4 RNA ligase 2, Taq DNA ligase, or E. coli DNA ligase.

In embodiments, ligating includes chemical ligation (e.g., enzyme-free, click-mediated ligation). In embodiments, the extension products include a first bioconjugate reactive moiety capable of bonding upon contact with a second (complementary) bioconjugate reactive moiety. In embodiments, the extension products include an alkynyl moiety at the 3′ and an azide moiety at the 5′ end that, upon hybridization to the target nucleic acid react to form a triazole linkage during suitable reaction conditions. Reaction conditions and protocols for chemical ligation techniques that are compatible with nucleic acid amplification methods are known in the art, for example, El-Sagheer, A. H., & Brown, T. (2012). Accounts of chemical research, 45(8), 1258-1267; Manuguerra I. et al. Chem Commun (Camb). 2018; 54(36):4529-4532; and Odeh, F., et al. (2019). Molecules (Basel, Switzerland), 25(1), 3, each of which are incorporated herein by reference in their entirety.

In embodiments, the methods of making integrated strand complements provided herein include interposing oligonucleotide probes according to any of the aspects or embodiments disclosed herein. In embodiments, the methods of making integrated strand complements described herein include interposing oligonucleotide probes that include a phosphorylated 5′ end. In embodiments, the methods of making integrated strand complements provided herein do not include interposing oligonucleotide probes with a phosphorylated 5′ end. In embodiments, the method includes phosphorylating the 5′ ends of the interposing probes prior to step (c). Phosphorylation may be performed, before, during, or after extension. In embodiments, phosphorylation occurs in parallel with the extension reaction. In embodiments, ligation reaction occurs in parallel with the extension reaction.

In embodiments, the immobilized primers are attached to the solid support at their 5′ ends. In embodiments, the immobilized primers attached to the solid support via a linker. The linker may also include spacer nucleotides. Including spacer nucleotides in the linker puts the polynucleotide in an environment having a greater resemblance to free solution. This can be beneficial, for example, in enzyme-mediated reactions such as sequencing-by-synthesis. It is believed that such reactions suffer less steric hindrance issues that can occur when the polynucleotide is directly attached to the solid support or is attached through a very short linker (e.g., a linker including about 1 to 3 carbon atoms). Spacer nucleotides form part of the polynucleotide but do not participate in any reaction carried out on or with the polynucleotide (e.g. a hybridization or amplification reaction). In embodiments, the spacer nucleotides include 1 to 20 nucleotides. In embodiments, the linker includes 10 spacer nucleotides. In embodiments, the linker includes 12 spacer nucleotides. In embodiments, the linker includes 15 spacer nucleotides. It is preferred to use polyT spacer nucleotides, although other nucleotides and combinations thereof can be used. In embodiments, the linker includes 10, 11, 12, 13, 14, or 15 dT spacer nucleotides. In embodiments, the linker includes 12 dT spacer nucleotides. Spacer nucleotides are typically included at the 5′ ends of polynucleotides which are attached to a suitable support. Attachment can be achieved via a phosphorothioate present at the 5′ end of the polynucleotide, an azide moiety, a dibenzocyclooctyne (DBCO) moiety, or any other bioconjugate reactive moiety. The linker may be a carbon-containing chain such as those of formula —(CH₂)n- wherein “n” is from 1 to about 1000. However, a variety of other linkers may be used so long as the linkers are stable under conditions used in DNA sequencing. In embodiments, the linker includes polyethylene glycol (PEG) having a general formula of —(CH₂—CH₂—O)m-, wherein m is from about 1 to 500. In embodiments, m is 8 to 24. In embodiments, m is 10 to 12. In embodiments, the linker, or the immobilized oligonucleotides (e.g., primers) include a cleavable site. In embodiments, a cleavable site is a location which allows controlled cleavage of the immobilized polynucleotide strand (e.g., the linker, the primer, or the polynucleotide) by chemical, enzymatic or photochemical means. In embodiments, the cleavable site includes one or more deoxyuracil nucleobases (dUTPs).

In embodiments, the immobilized primers are covalently attached to the solid support. In embodiments, the 5′ end of the immobilized primers contains a reacted functional group that served to tether the immobilized primers to the solid support (e.g., a bioconjugate linker). Non-limiting examples of covalent attachment include amine-modified polynucleotides reacting with epoxy or isothiocyanate groups on the solid support, succinylated polynucleotides reacting with aminophenyl or aminopropyl functional groups on the solid support, dibenzocyclooctyne-modified polynucleotides reacting with azide functional groups on the solid support (or vice versa), trans-cyclooctyne-modified polynucleotides reacting with tetrazine or methyl tetrazine groups on the solid support (or vice versa), disulfide modified polynucleotides reacting with mercapto-functional groups on the solid support, amine-functionalized polynucleotides reacting with carboxylic acid groups via 1-ethyl-3-(3-dimethylaminopropyl)-carbodiimide hydrochloride (EDC) chemistry, thiol-modified polynucleotides attaching to a solid support via a disulfide bond or maleimide linkage, alkyne-modified polynucleotides attaching to a solid support via copper-catalyzed click reactions to azide functional groups on the solid support, and acrydite-modified polynucleotides polymerizing with free acrylic acid monomers on the solid support to form polyacrylamide or reacting with thiol groups on the solid support. In embodiments, the primer is attached to the solid support polymer through electrostatic binding. For example, the negatively charged phosphate backbone of the primer may be bound electrostatically to positively charged monomers in the solid support.

In embodiments, each of the plurality of immobilized oligonucleotides (e.g., immobilized primers) is about 5 to about 25 nucleotides in length. In embodiments, each of the plurality of immobilized oligonucleotides (e.g., immobilized primers) is about 10 to about 40 nucleotides in length. In embodiments, each of the plurality of immobilized oligonucleotides (e.g., immobilized primers) is about 5 to about 100 nucleotides in length. In embodiments, each of the plurality of immobilized oligonucleotides (e.g., immobilized primers) is about 20 to 200 nucleotides in length. In embodiments, each of the plurality of immobilized oligonucleotides (e.g., immobilized primers) about or at least about 5, 6, 7, 8, 9, 10, 12, 15, 18, 20, 25, 30, 35, 40, 50 or more nucleotides in length.

In embodiments, the immobilized oligonucleotides include one or more phosphorothioate nucleotides. In embodiments, the immobilized oligonucleotides include a plurality of phosphorothioate nucleotides. In embodiments, about or at least about 20%, 30%, 40%, 50%, 60%, 70%, 80%, 90%, or about 100% of the nucleotides in the immobilized oligonucleotides are phosphorothioate nucleotides. In embodiments, most of the nucleotides in the immobilized oligonucleotides are phosphorothioate nucleotides. In embodiments, all of the nucleotides in the immobilized oligonucleotides are phosphorothioate nucleotides. In embodiments, none of the nucleotides in the immobilized oligonucleotides are phosphorothioate nucleotides. In embodiments, the 5′ end of the immobilized oligonucleotide includes one or more phosphorothioate nucleotides. In embodiments, the 5′ end of the immobilized oligonucleotide includes between one and five phosphorothioate nucleotides.

In embodiments, the template nucleic acid includes a first adapter and a second adapter, wherein the first adapter is a Y-adapter, a hairpin adapter, a blunt-ended adapter, or an adapter including a single-strand overhang and the second adapter is a Y-adapter, a hairpin adapter, a blunt-ended adapter, or an adapter including a single-strand overhang. In embodiments, the template nucleic acid includes a first adapter and a second adapter, wherein the first adapter is a Y-adapter and the second adapter is a Y-adapter. In embodiments, the template nucleic acid includes a first adapter and a second adapter, wherein the first adapter is a Y-adapter and the second adapter is a hairpin adapter. In embodiments, the template nucleic acid includes a first adapter and a second adapter, wherein the first adapter is a hairpin adapter and the second adapter is a Y-adapter. In embodiments, the template nucleic acid includes a first adapter and a second adapter, wherein the first adapter is a hairpin adapter and the second adapter is a hairpin adapter.

In some embodiments, the adapter is a Y-adapter. In embodiments, a Y-adapter includes a first strand and a second strand where a portion of the first strand (e.g., 3′-portion) is complementary, or substantially complementary, to a portion (e.g., 5′-portion) of the second strand. In embodiments, a Y-adapter includes a first strand and a second strand where a 3′-portion of the first strand is hybridized to a 5′-portion of the second strand. In embodiments, the 3′-portion of the first strand that is substantially complementary to the 5′-portion of the second strand forms a duplex including double stranded nucleic acid. Accordingly, a Y-adapter often includes a first end including a duplex region including a double stranded nucleic acid, and a second end including a forked region including a 5′-arm and a 3′-arm. In some embodiments, a 5′-portion of the first stand (e.g., 5′-arm) and a 3′-portion of the second strand (3′-arm) are not complementary. In embodiments, the first and second strands of a Y-adapter are not covalently attached to each other. In embodiments, the Y-adapter includes (i) a first strand having a 5′-arm and a 3′-portion, and (ii) a second strand having a 3′-arm and a 5′-portion, wherein the 3′-portion of the first strand is substantially complementary to the 5′-portion of the second strand, and the 5′-arm of the first strand is not substantially complementary to the 3′-arm of the second strand. In some embodiments, the first adapter includes a sample barcode sequence, a molecular identifier sequence, or both a sample barcode sequence and a molecular identifier sequence. In some embodiments, the first adapter includes a sample barcode sequence (e.g., a 6-10 nucleotide sequence).

In embodiments, ligating includes ligating both the 3′ end and the 5′ end of the duplex region of the first adapter to a double stranded nucleic acid. In embodiments, ligating includes ligating either the 3′ end or the 5′ end of the duplex region of the first adapter to a double stranded nucleic acid. In embodiments, ligating includes ligating the 5′ end of the duplex region of the first adapter to the double stranded nucleic acid and not the 3′ end of the duplex region. In embodiments, the method includes ligating a first adapter to a first end of the double stranded nucleic acid wherein both strands of the double stranded nucleic acid are ligated to the first adapter. In embodiments, the method includes ligating a first adapter to a first end of the double stranded nucleic acid wherein one strand of the double stranded nucleic acid is ligated to the first adapter.

In some embodiments, each strand of a Y-adapter, each of the non-complementary arms of a Y-adapter, or a duplex portion of a Y-adapter has a length independently selected from at least 5, at least 10, at least 15, at least 25, and at least 40 nucleotides. In some embodiments, each strand of a Y-adapter, each of the non-complementary arms of a Y-adapter, or a duplex portion of a Y-adapter has a length in a range independently selected from 15 to 500 nucleotides, 15-250 nucleotides, 15 to 200 nucleotides, 15 to 150 nucleotides, 20 to 100 nucleotides, 20 to 50 nucleotides and 10-50 nucleotides. In embodiments, one or both non-complementary arms of the Y-adapter is about or at least about 10, 15, 20, 25, 30, 35, 40, 45, 50, or more nucleotides in length. In embodiments, one or both non-complementary arms of the Y-adapter is about or at least about 20 nucleotides in length. In embodiments, one or both non-complementary arms of the Y-adapter is about or at least about 30 nucleotides in length. In embodiments, one or both non-complementary arms of the Y-adapter is about or at least about 40 nucleotides in length. In embodiments, the duplex portion of a Y-adapter is about or at least about 5, 10, 15, 20, 25, 30, or more nucleotides in length. In embodiments, the duplex portion of a Y-adapter is about 5-50, 5-25, or 10-15 nucleotides in length. In embodiments, the duplex portion of a Y-adapter is about or at least about 10 nucleotides in length. In embodiments, the duplex portion of a Y-adapter is about or at least about 15 nucleotides in length. In embodiments, the duplex portion of a Y-adapter is about or at least about 12 nucleotides in length. In embodiments, the duplex portion of a Y-adapter is about or at least about 20 nucleotides in length.

In some embodiments, a Y-adapter includes a first end including a duplex region including a double stranded nucleic acid, and a second end including a forked region, where the first end is configured for ligation to an end of a double stranded nucleic acid (e.g., a nucleic acid fragment, e.g., a library insert). In embodiments, a duplex end of a Y-adapter includes a 5′-overhang or a 3′-overhang that is complementary to a 3′-overhang or a 5′-overhang of an end of a double stranded nucleic acid. In some embodiments, a duplex end of a Y-adapter includes a blunt end that can be ligated to a blunt end of a double stranded nucleic acid. In certain embodiment, a duplex end of a Y-adapter includes a 5′-end that is phosphorylated.

In some embodiments, the first and/or second adapter (e.g., one or both strands of a Y-adapter) include one or more of a primer binding site, a capture nucleic acid binding site (e.g., a nucleic acid sequence complementary to a capture nucleic acid), a UMI, a sample barcode, a sequencing adapter, a label, a binding motif, the like or combinations thereof. In some embodiments, a non-complementary portion (e.g., 5′-arm and/or 3′-arm) of a Y-adapter includes one or more of a primer binding site, a capture nucleic acid binding site (e.g., a nucleic acid sequence complementary to a capture nucleic acid), a UMI, a sample barcode, a sequencing adapter, a label, a binding motif, the like or combinations thereof. In certain embodiments, a non-complementary portion of a Y-adapter includes a primer binding site. In certain embodiments, a non-complementary portion of a Y-adapter includes a binding site for a capture nucleic acid. In certain embodiments, a non-complementary portion of a Y-adapter includes a primer binding site and a UMI. In certain embodiments, a non-complementary portion of a Y-adapter includes a binding motif. In embodiments, the first and/or second adapter (e.g., one or both strands of a Y-adapter) does not include a UMI or sample barcode.

In embodiments, a complementary strand (e.g., a 3′-portion or 5′-portion) of a Y-adapter includes a primer binding site. In certain embodiments, a complementary strand (e.g., a 3′-portion or 5′-portion) of a Y-adapter includes a binding site for a capture nucleic acid. In certain embodiments, a complementary strand (e.g., a 3′-portion or 5′-portion) of a Y-adapter includes a primer binding site and a UMI. In certain embodiments, a complementary strand (e.g., a 3′-portion or 5′-portion) of a Y-adapter includes a binding motif.

In some embodiments, each of the non-complementary portions (i.e., arms) of a Y-adapter independently have a predicted, calculated, mean, average or absolute melting temperature (Tm) that is greater than 50° C., greater than 55° C., greater than 60° C., greater than 65° C., greater than 70° C. or greater than 75° C. In some embodiments, each of the non-complementary portions of a Y-adapter independently have a predicted, estimated, calculated, mean, average or absolute melting temperature (Tm) that is in a range of 50-100° C., 55-100° C., 60-100° C., 65-100° C., 70-100° C., 55-95° C., 65-95° C., 70-95° C., 55-90° C., 65-90° C., 70-90° C., or 60-85° C. In embodiments, the Tm is about or at least about 70° C. In embodiments, the Tm is about or at least about 75° C. In embodiments, the Tm is about or at least about 80° C. In embodiments, the Tm is a calculated Tm. Tm's are routinely calculated by those skilled in the art, such as by commercial providers of custom oligonucleotides. In embodiments, the Tm for a given sequence is determined based on that sequence as an independent oligo. In embodiments, Tm is calculated using web-based algorithms, such as Primer3 and Primer3Plus (www.bioinformatics.nl/cgi-bin/primer3plus/primer3plus.cgi) using default parameters. The Tm of a non-complementary portion of a Y-adapter can be changed (e.g., increased) to a desired Tm using a suitable method, for example by changing (e.g., increasing) GC content, changing (e.g., increasing) length and/or by the inclusion of modified nucleotides, nucleotide analogues and/or modified nucleotides bonds, non-limiting examples of which include locked nucleic acids (LNAs, e.g., bicyclic nucleic acids), bridged nucleic acids (BNAs, e.g., constrained nucleic acids), C5-modified pyrimidine bases (for example, 5-methyl-dC, propynyl pyrimidines, among others) and alternate backbone chemistries, for example peptide nucleic acids (PNAs), morpholinos, the like or combinations thereof. Accordingly, in some embodiments, each of the non-complementary portion of a Y-adapter independently includes one or more modified nucleotides, nucleotide analogues and/or modified nucleotides bonds.

In some embodiments, each of the non-complementary portions of a Y-adapter independently includes a GC content of greater than 40%, greater than 50%, greater than 55%, greater than 60% greater than 65% or greater than 70%. In certain embodiments, each of the non-complementary portions of a Y-adapter independently includes a GC content in a range of 40-100%, 50-100%, 60-100% or 70-100%. In embodiments, one or both non-complementary portions of a Y-adapter have a GC content of about or more than about 40%. In embodiments, one or both non-complementary portions of a Y-adapter have a GC content of about or more than about 50%. In embodiments, one or both non-complementary portions of a Y-adapter have a GC content of about or more than about 60%. Non-base modifiers can also be incorporated into a non-complementary portion of a Y-adapter to increase Tm, non-limiting examples of which include a minor grove binder (MGB), spermine, G-clamp, a Uaq anthraquinone cap, the like or combinations thereof.

In certain embodiments, a duplex region of a Y-adapter includes a predicted, estimated, calculated, mean, average or absolute Tm in a range of 30-70° C., 35-65° C., 35-60° C., 40-65° C., 40-60° C., 35-55° C., 40-55° C., 45-50° C. or 40-50° C. In embodiments, the Tm of a duplex region of the Y-adapter is about or more than about 30° C. In embodiments, the Tm of a duplex region of the Y-adapter is about or more than about 35° C. In embodiments, the Tm of a duplex region of the Y-adapter is about or more than about 40° C. In embodiments, the Tm of a duplex region of the Y-adapter is about or more than about 45° C. In embodiments, the Tm of a duplex region of the Y-adapter is about or more than about 50° C.

In some embodiments, the adapter is hairpin adapter. In embodiments, a hairpin adapter includes a single nucleic acid strand including a stem-loop structure. A hairpin adapter can be any suitable length. In some embodiments, a hairpin adapter is at least 40, at least 50, or at least 100 nucleotides in length. In some embodiments, a hairpin adapter has a length in a range of 45 to 500 nucleotides, 75-500 nucleotides, 45 to 250 nucleotides, 60 to 250 nucleotides or 45 to 150 nucleotides. In some embodiments, a hairpin adapter includes a nucleic acid having a 5′-end, a 5′-portion, a loop, a 3′-portion and a 3′-end (e.g., arranged in a 5′ to 3′ orientation). In some embodiments, the 5′ portion of a hairpin adapter is annealed and/or hybridized to the 3′ portion of the hairpin adapter, thereby forming a stem portion of the hairpin adapter. In some embodiments, the 5′ portion of a hairpin adapter is substantially complementary to the 3′ portion of the hairpin adapter. In certain embodiments, a hairpin adapter includes a stem portion (i.e., stem) and a loop, wherein the stem portion is substantially double stranded thereby forming a duplex. In some embodiments, the loop of a hairpin adapter includes a nucleic acid strand that is not complementary (e.g., not substantially complementary) to itself or to any other portion of the hairpin adapter. In some embodiments, the second adapter includes a sample barcode sequence, a molecular identifier sequence, or both a sample barcode sequence and a molecular identifier sequence. In some embodiments, the second adapter includes a sample barcode sequence.

In some embodiments, a duplex region or stem portion of a hairpin adapter includes an end that is configured for ligation to an end of double stranded nucleic acid (e.g., a nucleic acid fragment, e.g., a library insert). In embodiments, an end of a duplex region or stem portion of a hairpin adapter includes a 5′-overhang or a 3′-overhang that is complementary to a 3′-overhang or a 5′-overhang of one end of a double stranded nucleic acid. In some embodiments, an end of a duplex region or stem portion of a hairpin adapter includes a blunt end that can be ligated to a blunt end of a double stranded nucleic acid. In certain embodiment, an end of a duplex region or stem portion of a hairpin adapter includes a 5′-end that is phosphorylated. In some embodiments, a stem portion of a hairpin adapter is at least 15, at least 25, or at least 40 nucleotides in length. In some embodiments, a stem portion of a hairpin adapter has a length in a range of 15 to 500 nucleotides, 15-250 nucleotides, 15 to 200 nucleotides, 15 to 150 nucleotides, 20 to 100 nucleotides or 20 to 50 nucleotides.

In embodiments, ligating includes ligating both the 3′ end and the 5′ end of the duplex region of the second adapter to the double stranded nucleic acid. In embodiments, ligating includes ligating either the 3′ end or the 5′ end of the duplex region of the second adapter to the double stranded nucleic acid. In embodiments, ligating includes ligating the 5′ end of the duplex region of the second adapter to the double stranded nucleic acid and not the 3′ end of the duplex region.

In some embodiments, the loop of a hairpin adapter includes one or more of the following: a primer binding site, a capture nucleic acid binding site (e.g., a nucleic acid sequence complementary to a capture nucleic acid), a UMI, a sample barcode, a sequencing adapter, a label, the like or combinations thereof. In certain embodiments, a loop of a hairpin adapter includes a primer binding site. In certain embodiments, a loop of a hairpin adapter includes a primer binding site and a UMI. In certain embodiments, a loop of a hairpin adapter includes a binding motif.

In some embodiments, the loop of a hairpin adapter has a predicted, calculated, mean, average or absolute melting temperature (Tm) that is greater than 50° C., greater than 55° C., greater than 60° C., greater than 65° C., greater than 70° C. or greater than 75° C. In some embodiments, a loop of a hairpin adapter has a predicted, estimated, calculated, mean, average or absolute melting temperature (Tm) that is in a range of 50-100° C., 55-100° C., 60-100° C., 65-100° C., 70-100° C., 55-95° C., 65-95° C., 70-95° C., 55-90° C., 65-90° C., 70-90° C., or 60-85° C. In embodiments, the Tm of the loop is about 65° C. In embodiments, the Tm of the loop is about 75° C. In embodiments, the Tm of the loop is about 85° C. The Tm of a loop of a hairpin adapter can be changed (e.g., increased) to a desired Tm using a suitable method, for example by changing (e.g., increasing GC content), changing (e.g., increasing) length and/or by the inclusion of modified nucleotides, nucleotide analogues and/or modified nucleotides bonds, non-limiting examples of which include locked nucleic acids (LNAs, e.g., bicyclic nucleic acids), bridged nucleic acids (BNAs, e.g., constrained nucleic acids), C5-modified pyrimidine bases (for example, 5-methyl-dC, propynyl pyrimidines, among others) and alternate backbone chemistries, for example peptide nucleic acids (PNAs), morpholinos, the like or combinations thereof. Accordingly, in some embodiments, a loop of a hairpin adapter includes one or more modified nucleotides, nucleotide analogues and/or modified nucleotides bonds.

In some embodiments, the loop of a hairpin adapter independently includes a GC content of greater than 40%, greater than 50%, greater than 55%, greater than 60% greater than 65% or greater than 70%. In certain embodiments, a loop of a hairpin adapter independently includes a GC content in a range of 40-100%, 50-100%, 60-100% or 70-100%. In embodiments, the loop has a GC content of about or more than about 40%. In embodiments, the loop has a GC content of about or more than about 50%. In embodiments, the loop has a GC content of about or more than about 60%. Non-base modifiers can also be incorporated into a loop of a hairpin adapter to increase Tm, non-limiting examples of which include a minor grove binder (MGB), spermine, G-clamp, a Uaq anthraquinone cap, the like or combinations thereof. A loop of a hairpin adapter can be any suitable length. In some embodiments, a loop of a hairpin adapter is at least 15, at least 25, or at least 40 nucleotides in length. In some embodiments, a hairpin adapter has a length in a range of 15 to 500 nucleotides, 15-250 nucleotides, 20 to 200 nucleotides, 30 to 150 nucleotides or 50 to 100 nucleotides.

In certain embodiments, a duplex region or stem region of a hairpin adapter includes a predicted, estimated, calculated, mean, average or absolute Tm in a range of 30-70° C., 35-65° C., 35-60° C., 40-65° C., 40-60° C., 35-55° C., 40-55° C., 45-50° C. or 40-50° C. In embodiments, the Tm of the stem region is about or more than about 35° C. In embodiments, the Tm of the stem region is about or more than about 40° C. In embodiments, the Tm of the stem region is about or more than about 45° C. In embodiments, the Tm of the stem region is about or more than about 50° C.

In embodiments, the template nucleic acid is a double-stranded polynucleotide. In embodiments, the double-stranded polynucleotide includes genomic DNA, complementary DNA (cDNA), or cell-free DNA (cfDNA). In embodiments, the template nucleic acid is a single-stranded polynucleotide. In embodiments, the single stranded polynucleotide includes messenger RNA (mRNA), transfer RNA (tRNA), ribosomal RNA (rRNA), cell-free RNA (cfRNA), or noncoding RNA (ncRNA).

In embodiments, the double-stranded polynucleotide is about 100 to 1000 nucleotides in length. In embodiments, the double-stranded polynucleotide is about 350 nucleotides in length. In embodiments, the double-stranded polynucleotide is about 10, 20, 50, 100, 150, 200, 300, or 500 nucleotides in length. The double-stranded polynucleotide molecules can vary length, such as about 100-300 nucleotides long, about 300-500 nucleotides long, or about 500-1000 nucleotides long. In embodiments, the double-stranded polynucleotide molecular is about 100-1000 nucleotides, about 150-950 nucleotides, about 200-900 nucleotides, about 250-850 nucleotides, about 300-800 nucleotides, about 350-750 nucleotides, about 400-700 nucleotides, or about 450-650 nucleotides. In embodiments, the double-stranded polynucleotide molecule is about 150 nucleotides. In embodiments, the double-stranded polynucleotide is about 100-1000 nucleotides long. In embodiments, the double-stranded polynucleotide is about 100-300 nucleotides long. In embodiments, the double-stranded polynucleotide is about 300-500 nucleotides long. In embodiments, the double-stranded polynucleotide is about 500-1000 nucleotides long. In embodiments, the double-stranded polynucleotide molecule is about 100 nucleotides. In embodiments, the double-stranded polynucleotide molecule is about 300 nucleotides. In embodiments, the double-stranded polynucleotide molecule is about 500 nucleotides. In embodiments, the double-stranded polynucleotide molecule is about 1000 nucleotides.

In embodiments the double-stranded polynucleotide (e.g., genomic template DNA) is first treated to form single-stranded linear nucleic acid fragments (e.g., ranging in length from about 50 to about 600 nucleotides). Treatment typically entails fragmentation, such as by chemical fragmentation, enzymatic fragmentation, or mechanical fragmentation, followed by denaturation to produce single-stranded DNA fragments. In embodiments, the double-stranded polynucleotide includes an adapter. The adapter may have other functional elements including tagging sequences (i.e., a barcode), attachment sequences, palindromic sequences, restriction sites, sequencing primer binding sites, functionalization sequences, and the like. Barcodes can be of any of a variety of lengths. In embodiments, the primer includes a barcode that is 10-50, 20-30, or 4-12 nucleotides in length. In embodiments, the adapter includes a primer binding sequence that is complementary to at least a portion of a primer (e.g., a sequencing primer). Primer binding sites can be of any suitable length. In embodiments, a primer binding site is about or at least about 10, 15, 20, 25, 30, or more nucleotides in length. In embodiments, a primer binding site is 10-50, 15-30, or 20-25 nucleotides in length. In embodiments the double-stranded polynucleotide is cfDNA.

In embodiments, the double-stranded polynucleotide includes known adapter sequences on the 5′ and 3′ ends.

In embodiments, forming a plurality of amplification products includes bridge polymerase chain reaction (bPCR) amplification, solid-phase rolling circle amplification (RCA), solid-phase exponential rolling circle amplification (eRCA), solid-phase recombinase polymerase amplification (RPA), solid-phase helicase dependent amplification (HDA), template walking amplification, or emulsion PCR on particles, or combinations of the methods. In embodiments, generating a double-stranded amplification product includes a bridge polymerase chain reaction (bPCR) amplification. In embodiments, generating a double-stranded amplification product includes a thermal bridge polymerase chain reaction (t-bPCR) amplification. In embodiments, generating a double-stranded amplification product includes a chemical bridge polymerase chain reaction (c-bPCR) amplification. Chemical bridge polymerase chain reactions include fluidically cycling a denaturant (e.g., formamide) and maintaining the temperature within a narrow temperature range (e.g., +/−5° C.). In contrast, thermal bridge polymerase chain reactions include thermally cycling between high temperatures (e.g., 85° C.-95° C.) and low temperatures (e.g., 60° C.-70° C.). Thermal bridge polymerase chain reactions may also include a denaturant, typically at a much lower concentration than traditional chemical bridge polymerase chain reactions.

In embodiments, forming a plurality of amplification products includes bridge amplification; for example, as exemplified by the disclosures of U.S. Pat. Nos. 5,641,658; 7,115,400; 7,790,418; U.S. Patent Publ. No. 2008/0009420, each of which is incorporated herein by reference in its entirety. In general, bridge amplification uses repeated steps of annealing of primers to templates, primer extension, and separation of extended primers from templates. Because the forward and reverse primers are attached to the solid support, the extension products released upon separation from an initial template are also attached to the solid support. Both strands are immobilized on the solid support at the 5′ end, preferably via a covalent attachment. The 3′ end of an amplification product is then permitted to anneal to a nearby reverse primer, forming a “bridge” structure. The reverse primer is then extended to produce a further template molecule that can form another bridge. During bridge PCR, additional chemical additives may be included in the reaction mixture, in which the DNA strands are denatured by flowing a denaturant over the DNA, which chemically denatures complementary strands. This is followed by washing out the denaturant and reintroducing a polymerase in buffer conditions that allow primer annealing and extension.

In embodiments, forming a plurality of amplification products includes amplifying the template polynucleotide or complement thereof on a solid support including a plurality of primers attached to the solid support, wherein the plurality of primers include a plurality of forward primers with complementarity to the template polynucleotide and a plurality of reverse primers with complementarity to a complement of the template polynucleotide, and the amplifying includes a plurality of cycles of strand denaturation, primer hybridization, and primer extension.

In embodiments, the plurality of strand denaturation cycles are different for one or more cycles, wherein the initial denaturation cycle is maintained at different conditions from the remaining denaturation cycles. For example, in embodiments, the initial denaturation cycle is at about 85° C.-95° C. for about 1 minute to about 10 minutes, whereas denaturation in the remaining cycles is different (e.g., about 85° C. for about 15-30 sec). In embodiments, the initial denaturation is maintained at about 85° C.-95° C. for about 5 minutes to about 10 minutes. In embodiments, the initial denaturation is maintained at 90° C.-95° C. for about 1 to 10 minutes. In embodiments, the initial denaturation is maintained at 80° C.-85° C. for about 1 to 10 minutes. In embodiments, the initial denaturation is maintained at 85° C.-90° C. for about 1 to 10 minutes. In embodiments, the initial denaturation is maintained at about 85° C.-95° C. for about 1 minutes to about 10 minutes. In embodiments, the initial denaturation is maintained at about 95° C. for about 5 minutes to about 10 minutes. In embodiments, the initial denaturation is maintained at about 85° C.-95° C. for about 5 minutes to about 10 minutes.

In embodiments, forming a plurality of amplification products includes a thermal bridge polymerase chain reaction (t-bPCR) amplification. In embodiments, the plurality of cycles includes thermally cycling between (i) about 85° C. for about 15-30 sec for denaturation, and (ii) about 65° C. for about 1 minute for annealing/extension of the primer. In embodiments, the plurality of cycles includes thermally cycling between (i) about 85° C. for about 15-30 sec for denaturation, and (ii) about 65° C. for about 30 seconds for annealing/extension of the primer.

In embodiments, forming a plurality of amplification products includes chemical bridge polymerase chain reaction (c-bPCR) amplification. In embodiments, forming a plurality of amplification products includes denaturation using a chemical denaturant. In embodiments, forming a plurality of amplification products includes denaturation using acetic acid, hydrochloric acid, nitric acid, formamide, guanidine, sodium salicylate, sodium hydroxide, dimethyl sulfoxide (DMSO), propylene glycol, urea, or a mixture thereof. In embodiments, the chemical denaturant is sodium hydroxide or formamide. In embodiments, forming a plurality of amplification products includes thermal bridge polymerase chain reaction (t-bPCR) amplification. In embodiments, forming a plurality of amplification products includes chemical bridge polymerase chain reaction (c-bPCR) amplification. Chemical bridge polymerase chain reactions include fluidically cycling a denaturant (e.g., formamide) and maintaining the temperature within a narrow temperature range (e.g., +/−5° C.). In contrast, thermal bridge polymerase chain reactions include thermally cycling between high temperatures (e.g., 85° C.-95° C.) and low temperatures (e.g., 60° C.-70° C.). Thermal bridge polymerase chain reactions may also include a denaturant, typically at a significantly lower concentration than traditional chemical bridge polymerase chain reactions.

In embodiments, forming a plurality of amplification products includes fluidic cycling between an extension mixture that includes a polymerase and dNTPs, and a chemical denaturant. In embodiments, the polymerase is a strand-displacing polymerase or a non-strand displacing polymerase. In embodiments, the solutions are thermally cycled between about 40° C. to about 65° C. during fluidic cycling of the extension mixture and the chemical denaturant. For example, the extension cycle is maintained at a temperature of 55° C.-65° C., followed by a denaturation cycle that is maintained at a temperature of 40° C.-65° C., or by a denaturation step in which the temperature starts at 60° C.-65° C. and is ramped down to 40° C. prior to exchanging the reagent. In embodiments, step (b) includes modulating the reaction temperature prior to initiating the next cycle. In embodiments, the denaturation cycle and/or the extension cycle is maintained at a temperature for a sufficient amount of time, and prior to starting the next cycle the temperature is modulated (e.g., increased relative to the starting temperature or reduced relative to the starting temperature). In embodiments, the denaturation cycle is performed at a temperature of 60° C.-65° C. for about 5-45 sec, then the temperature is reduced (e.g., lowered to about 40° C.) before starting an extension cycle (i.e., before introducing an extension mixture). Lowering the temperature, even in the presence of a chemical denaturant, facilitates primer hybridization in the subsequent step when the amplicons are exposed to conditions that promote hybridization. In embodiments, the extension cycle is performed at a temperature of 50° C.-60° C. for about 0.5-2 minutes, then the temperature is increased (e.g., raised to between about 60° C. to about 70° C., or to about 65° C. to about 72° C.) after introducing the extension mixture. In embodiments, the cycling between the extension mixture and the chemical denaturant is performed at least 5, at least 10, at least 20, at least 30, at least 40, at least 50, at least 75, at least 100, or at least 200 times. In embodiments, the cycling between the extension mixture and the chemical denaturant is performed about 5, about 10, about 20, about 30, about 40, about 50, about 75, about 100, or about 200 times. In embodiments, the cycling between the extension mixture and the chemical denaturant is performed a total of 5, 10, 20, 30, 40, 50, 75, 100, 200, or more times. In embodiments, the fluidic cycling is performed in the presence of about 2 to about 15 mM Mg²⁺. In embodiments, the fluidic cycling is performed in the presence of about 2, about 3, about 4, about 5, about 6, about 7, about 8, about 9, about 10, about 11, about 12, about 13, about 14, or about 15 mM Mg²⁺.

In embodiments, forming a plurality of amplification products includes a plurality of strand denaturation cycles, wherein the initial denaturation cycle is at different conditions from the remaining denaturation cycles. For example, in embodiments, the initial denaturation cycle is at about 85° C.-95° C. for about 1 minute to about 10 minutes, whereas denaturation in the remaining cycles is different (e.g., about 85° C. for about 15-30 sec). In embodiments, forming a plurality of amplification products includes an initial denaturation at about 85° C.-95° C. for about 5 minutes to about 10 minutes. In embodiments, forming a plurality of amplification products includes an initial denaturation at 90° C.-95° C. for about 1 to 10 minutes. In embodiments, forming a plurality of amplification products includes an initial denaturation at 80° C.-85° C. for about 1 to 10 minutes. In embodiments, forming a plurality of amplification products includes an initial denaturation at 85° C.-90° C. for about 1 to 10 minutes.

In embodiments, the plurality of cycles includes thermally cycling between (i) about 80° C. to 90° C. for denaturation, and (ii) about 55° C. to about 65° C. for annealing/extension of the primer. In embodiments, the plurality of cycles includes thermally cycling between (i) about 85° C. for denaturation, and (ii) about 55° C. for annealing/extension of the primer. In embodiments, the plurality of cycles includes thermally cycling between (i) about 85° C. for denaturation, and (ii) about 65° C. for annealing/extension of the primer. In embodiments, the plurality of cycles includes thermally cycling between (i) less than 80° C. (e.g., 70 to 80° C.) for denaturation, and (ii) about 55° C. to about 65° C. for annealing/extension of the primer. In embodiments, the plurality of cycles includes thermally cycling between (i) about 70° C. for denaturation, and (ii) about 65° C. for annealing/extension of the primer. In embodiments, the plurality of cycles includes thermally cycling between (i) about 75° C. for denaturation, and (ii) about 55° C. for annealing/extension of the primer. In embodiments, the plurality of cycles includes thermally cycling between (i) about 85° C. for denaturation, and (ii) about 65° C. for annealing/extension of the primer.

In embodiments, the plurality of cycles includes thermally cycling between (i) about 85° C. for less than 1 minute for denaturation, and (ii) about 65° C. for about 1 to 2 minutes for annealing/extension of the primer. In embodiments, the plurality of cycles includes thermally cycling between (i) about 85° C. for less than 1 minute for denaturation, and (ii) about 60° C. to about 65° C. for about 1 minute for annealing/extension of the primer. In embodiments, the plurality of cycles includes thermally cycling between (i) about 85° C. for about 15-30 sec for denaturation and (ii) about 65° C. for about 1 minute for annealing/extension of the primer. In embodiments, the plurality of cycles includes thermally cycling between (i) about 85° C. for about 30 sec for denaturation and (ii) about 65° C. for about 1 minute for annealing/extension of the primer. In embodiments, the plurality of cycles includes thermally cycling between (i) about 85° C. for about 15-30 sec for denaturation, and (ii) about 65° C. for about 30 seconds for annealing/extension of the primer. In embodiments, the plurality of cycles includes thermally cycling between (i) about 85° C. for about 15-30 sec for denaturation, and (ii) about 65° C. for about 1 minute for annealing/extension of the primer.

In embodiments, the plurality of denaturation steps is at a temperature of about 80° C.-95° C. In embodiments, the plurality of denaturation steps is at a temperature of about 80° C.-90° C. In embodiments, the plurality of denaturation steps is at a temperature of about 85° C.-90° C. In embodiments, the plurality of denaturation steps is at a temperature of about 81° C., 82° C., 83° C., 84° C., 85° C., 86° C., 87° C., 88° C., 89° C., or about 90° C. In embodiments, the plurality of denaturation steps is at a temperature of about 70° C.-85° C. In embodiments, the plurality of denaturation steps is at a temperature of about 70° C.-80° C. In embodiments, the plurality of denaturation steps is at a temperature of about 75° C.-80° C. In embodiments, the plurality of denaturation steps is at a temperature of about 70° C., 71° C., 72° C., 73° C., 74° C., 75° C., 76° C., 77° C., 78° C., 79° C., or about 80° C. In embodiments, the annealing/extension of the primer cycle is at a temperature of about 55° C., 56° C., 57° C., 58° C., 59° C., 60° C., 61° C., 62° C., 63° C., 64° C., or about 65° C.

In embodiments, the plurality of cycles includes thermally cycling between (i) about 80° C. to 90° C. for denaturation, and (ii) about 55° C. to about 65° C. for annealing/extension of the primer. In embodiments, the plurality of cycles includes thermally cycling between (i) about 85° C. for denaturation, and (ii) about 55° C. for annealing/extension of the primer. In embodiments, the plurality of cycles includes thermally cycling between (i) about 85° C. for denaturation, and (ii) about 65° C. for annealing/extension of the primer. In embodiments, the plurality of cycles includes thermally cycling between (i) less than 80° C. (e.g., 70 to 80° C.) for denaturation, and (ii) about 55° C. to about 65° C. for annealing/extension of the primer. In embodiments, the plurality of cycles includes thermally cycling between (i) about 70° C. for denaturation, and (ii) about 65° C. for annealing/extension of the primer. In embodiments, the plurality of cycles includes thermally cycling between (i) about 75° C. for denaturation, and (ii) about 55° C. for annealing/extension of the primer. In embodiments, the plurality of cycles includes thermally cycling between (i) about 85° C. for denaturation, and (ii) about 65° C. for annealing/extension of the primer.

In embodiments, the plurality of cycles includes thermally cycling between (i) about 85° C. for less than 1 minute for denaturation, and (ii) about 65° C. for about 1 to 2 minutes for annealing/extension of the primer. In embodiments, the plurality of cycles includes thermally cycling between (i) about 85° C. for less than 1 minute for denaturation, and (ii) about 60° C. to about 65° C. for about 1 minute for annealing/extension of the primer. In embodiments, the plurality of cycles includes thermally cycling between (i) about 85° C. for about 15-30 sec for denaturation and (ii) about 65° C. for about 1 minute for annealing/extension of the primer. In embodiments, the plurality of cycles includes thermally cycling between (i) about 85° C. for about 30 sec for denaturation and (ii) about 65° C. for about 1 minute for annealing/extension of the primer. In embodiments, the plurality of cycles includes thermally cycling between (i) about 85° C. for about 15-30 sec for denaturation, and (ii) about 65° C. for about 30 seconds for annealing/extension of the primer. In embodiments, the plurality of cycles includes thermally cycling between (i) about 85° C. for about 15-30 sec for denaturation, and (ii) about 65° C. for about 1 minute for annealing/extension of the primer. In embodiments, the temperature and duration for the annealing of the primer and the extension of the primer are different. In embodiments, the plurality of cycles includes thermally cycling between (i) about 90° C. to 95° C. for about 15 to 30 sec for denaturation and (ii) about 55° C. to about 65° C. for about 30 to 60 seconds for annealing and about 65° C. to 70° C. for about 30 to 60 seconds for extension of the primer. In embodiments, the plurality of denaturation steps is at a temperature of about 80° C.-95° C. In embodiments, the plurality of denaturation steps is at a temperature of about 80° C.-90° C. In embodiments, the plurality of denaturation steps is at a temperature of about 85° C.-90° C. In embodiments, the plurality of denaturation steps is at a temperature of about 81° C., 82° C., 83° C., 84° C., 85° C., 86° C., 87° C., 88° C., 89° C., or about 90° C. In embodiments, the plurality of denaturation steps is at a temperature of about 91° C., 92° C., 93° C., 94° C., 95° C., 96° C., 97° C., 98° C., or about 99° C. In embodiments, the plurality of denaturation steps is at a temperature of about 87° C., 88° C., 89° C., 90° C., 91° C., 92° C., 93° C., 94° C., or about 95° C. In embodiments, the plurality of denaturation steps is at a temperature of about 90° C., 91° C., 92° C., 93° C., 94° C., or about 95° C. In embodiments, the plurality of denaturation steps is at a temperature of about 70° C.-85° C. In embodiments, the plurality of denaturation steps is at a temperature of about 70° C.-80° C. In embodiments, the plurality of denaturation steps is at a temperature of about 75° C.-80° C. In embodiments, the plurality of denaturation steps is at a temperature of about 70° C., 71° C., 72° C., 73° C., 74° C., 75° C., 76° C., 77° C., 78° C., 79° C., or about 80° C. In embodiments, the annealing/extension of the primer cycle is at a temperature of about 55° C., 56° C., 57° C., 58° C., 59° C., 60° C., 61° C., 62° C., 63° C., 64° C., or about 65° C.

In embodiments, forming a plurality of amplification products includes incubation in a denaturant. In embodiments, the denaturant is acetic acid, ethylene glycol, hydrochloric acid, nitric acid, formamide, guanidine, sodium salicylate, sodium hydroxide, dimethyl sulfoxide (DMSO), propylene glycol, urea, or a mixture thereof. In embodiments, the denaturant is an additive that lowers a DNA denaturation temperature. In embodiments, the denaturant is betaine, dimethyl sulfoxide (DMSO), ethylene glycol, formamide, glycerol, guanidine thiocyanate, 4-methylmorpholine 4-oxide (NMO), or a mixture thereof. In embodiments, the denaturant is betaine, dimethyl sulfoxide (DMSO), ethylene glycol, formamide, glycerol, guanidine thiocyanate, or 4-methylmorpholine 4-oxide (NMO).

In embodiments, forming a plurality of amplification products includes a plurality of cycles of strand denaturation, primer hybridization, and primer extension. Although each cycle will include each of these three events (denaturation, hybridization, and extension), events within a cycle may or may not be discrete. For example, each step may have different reagents and/or reaction conditions (e.g., temperatures). Alternatively, some steps may proceed without a change in reaction conditions. For example, extension may proceed under the same conditions (e.g., same temperature) as hybridization. After extension, the conditions are changed to start a new cycle with a new denaturation step, thereby amplifying the amplicons. Primer extension products from an earlier cycle may serve as templates for a later amplification cycle. In embodiments, the plurality of cycles is about 5 to about 50 cycles. In embodiments, the plurality of cycles is about 10 to about 45 cycles. In embodiments, the plurality of cycles is about 10 to about 20 cycles. In embodiments, the plurality of cycles is about 20 to about 30 cycles. In embodiments, the plurality of cycles is 10 to 45 cycles. In embodiments, the plurality of cycles is 10 to 20 cycles. In embodiments, the plurality of cycles is 20 to 30 cycles. In embodiments, the plurality of cycles is about 10 to about 45 cycles. In embodiments, the plurality of cycles is about 20 to about 30 cycles.

In embodiments, forming a plurality of amplification products includes rolling circle amplification (RCA) (see, e.g., Lizardi et al., Nat. Genet. 19:225-232 (1998), which is incorporated herein by reference in its entirety). Several suitable RCA methods are known in the art. For example, RCA amplifies a circular polynucleotide (e.g., DNA) by polymerase extension of an amplification primer complementary to a portion of the template nucleic acid. This process generates copies of the circular polynucleotide template such that multiple complements of the template sequence arranged end to end in tandem are generated (i.e., a concatemer).

In embodiments, forming a plurality of amplification products includes exponential rolling circle amplification (eRCA). Exponential RCA is similar to the linear process except that it uses a second primer having a sequence that is identical to at least a portion of the circular template (Lizardi et al. Nat. Genet. 19:225 (1998)). This two-primer system achieves isothermal, exponential amplification. Exponential RCA has been applied to the amplification of non-circular DNA through the use of a linear probe that binds at both of its ends to contiguous regions of a target DNA followed by circularization using DNA ligase (Nilsson et al. Science 265(5181):208 5(1994)).

In embodiments, forming a plurality of amplification products includes hyperbranched rolling circle amplification (HRCA). Hyperbranched RCA uses a second primer complementary to the first amplification product. This allows products to be replicated by a strand-displacement mechanism, which can yield a drastic amplification within an isothermal reaction (Lage et al., Genome Research 13:294-307 (2003), which is incorporated herein by reference in its entirety).

In embodiments, the method includes amplifying a template nucleic acid by extending an amplification primer with a strand-displacing polymerase for about 10 seconds to about 30 minutes. In embodiments, the method includes amplifying a template nucleic acid by extending an amplification primer with a strand-displacing polymerase for about 30 seconds to about 16 minutes. In embodiments, the method includes amplifying a template nucleic acid by extending an amplification primer with a strand-displacing polymerase for about 30 seconds to about 10 minutes. In embodiments, the method includes amplifying a template nucleic acid by extending an amplification primer with a strand-displacing polymerase for about 30 seconds to about 5 minutes. In embodiments, the method includes amplifying a template nucleic acid by extending an amplification primer with a strand-displacing polymerase for about 1 second to about 5 minutes. In embodiments, the method includes amplifying a template nucleic acid by extending an amplification primer with a strand-displacing polymerase for about 1 second to about 2 minutes.

In embodiments, the method includes amplifying a template nucleic acid by extending an amplification primer with a strand-displacing polymerase at a temperature of about 20° C. to about 50° C. In embodiments, the method includes amplifying a template nucleic acid by extending an amplification primer with a strand-displacing polymerase at a temperature of about 30° C. to about 50° C. In embodiments, the method includes amplifying a template nucleic acid by extending an amplification primer with a strand-displacing polymerase at a temperature of about 25° C. to about 45° C. In embodiments, the method includes amplifying a template nucleic acid by extending an amplification primer with a strand-displacing polymerase at a temperature of about 35° C. to about 45° C. In embodiments, the method includes amplifying a template nucleic acid by extending an amplification primer with a strand-displacing polymerase at a temperature of about 35° C. to about 42° C. In embodiments, the method includes amplifying a template nucleic acid by extending an amplification primer with a strand-displacing polymerase at a temperature of about 37° C. to about 40° C.

In embodiments, the strand-displacing enzyme is an SD polymerase, Bst large fragment polymerase, or a phi29 polymerase or mutant thereof. In embodiments, the strand-displacing polymerase is phi29 polymerase, phi29 mutant polymerase or a thermostable phi29 mutant polymerase. A “phi29 polymerase” (or “Φ29 polymerase”) is a DNA polymerase from the (29 phage or from one of the related phages that, like Φ29, contain a terminal protein used in the initiation of DNA replication. For example, phi29 polymerases include the B103, GA-1, PZA, Φ15, BS32, M2Y (also known as M2), Nf, G1, Cp-1, PRD1, PZE, SFS, Cp-5, Cp-7, PR4, PR5, PR722, L17, Φ21, and AV-1 DNA polymerases, as well as chimeras thereof. A phi29 mutant DNA polymerase includes one or more mutations relative to naturally-occurring wild-type phi29 DNA polymerases, for example, one or more mutations that alter interaction with and/or incorporation of nucleotide analogs, increase stability, increase read length, enhance accuracy, increase photo-tolerance, and/or alter another polymerase property, and can include additional alterations or modifications over the wild-type phi29 DNA polymerase, such as one or more deletions, insertions, and/or fusions of additional peptide or protein sequences. Thermostable phi29 mutant polymerases are known in the art, see for example US 2014/0322759, which is incorporated herein by reference for all purposes. For example, a thermostable phi29 mutant polymerase refers to an isolated bacteriophage phi29 DNA polymerase including at least one mutation selected from the group consisting of M8R, V51A, M97T, L123S, G197D, K209E, E221K, E239G, Q497P, K512E, E515A, and F526 (relative to wild type phi29 polymerase).

In embodiments, the double-stranded amplification product is provided in a clustered array. In embodiments, the clustered array includes a plurality of double-stranded amplification products localized to discrete sites on a solid support. In embodiments, the solid support is a bead. In embodiments, the solid support is substantially planar. In embodiments, the solid support is contained within a flow cell.

In embodiments, the amplification primers are each attached to the solid support (i.e., immobilized on the surface of a solid support). The polynucleotide molecules can be fixed to surface by a variety of techniques, including covalent attachment and non-covalent attachment. In embodiments, the polynucleotides are confined to an area of a discrete region (referred to as a cluster). The discrete regions may have defined locations in a regular array, which may correspond to a rectilinear pattern, circular pattern, hexagonal pattern, or the like. A regular array of such regions is advantageous for detection and data analysis of signals collected from the arrays during an analysis. These discrete regions are separated by interstitial regions. As used herein, the term “interstitial region” refers to an area in a substrate or on a surface that separates other areas (e.g., overlapping clusters) of the substrate or surface. For example, an interstitial region can separate one concave feature of an array from another concave feature of the array. The two regions that are separated from each other can be discrete, lacking contact with each other. In another example, an interstitial region can separate a first portion of a feature from a second portion of a feature. In embodiments the interstitial region is continuous whereas the features are discrete, for example, as is the case for an array of wells in an otherwise continuous surface. The separation provided by an interstitial region can be partial or full separation. Interstitial regions will typically have a surface material that differs from the surface material of the features on the surface. For example, features of an array can have polynucleotides that exceeds the amount or concentration present at the interstitial regions. In some embodiments the polynucleotides and/or primers may not be present at the interstitial regions. In embodiments, at least two different primers are attached to the solid support (e.g., a forward and a reverse primer), which facilitates generating multiple amplification products from the first extension product or a complement thereof.

In embodiments, the clusters (e.g., overlapping clusters) have a mean or median separation from one another of about 0.5-5 μm. In embodiments, the mean or median separation is about 0.1-10 microns, 0.25-5 microns, 0.5-2 microns, 1 micron, or a number or a range between any two of these values. In embodiments, the mean or median separation is about or at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0 μm or a number or a range between any two of these values. In embodiments, the mean or median separation is about 0.1-10 microns. In embodiments, the mean or median separation is about 0.25-5 microns. In embodiments, the mean or median separation is about 0.5-2 microns. In embodiments, the mean or median separation is about or at least about 0.1 μm. In embodiments, the mean or median separation is about or at least about 0.25 μm. In embodiments, the mean or median separation is about or at least about 0.5 μm. In embodiments, the mean or median separation is about or at least about 1.0 μm. In embodiments, the mean or median separation is about or at least about 1.5 μm. In embodiments, the mean or median separation is about or at least about 2.0 μm. In embodiments, the mean or median separation is about or at least about 5.0 μm. In embodiments, the mean or median separation is about or at least about 10 μm. The mean or median separation may be measured center-to-center (i.e., the center of one cluster to the center of a second cluster). In embodiments of the methods provided herein, the amplicon clusters have a mean or median separation (measured center-to-center) from one another of about 0.5-5 μm. The mean or median separation may be measured edge-to-edge (i.e., the edge of one amplicon cluster to the edge of a second amplicon cluster). In embodiments of the methods provided herein, the amplicon clusters have a mean or median separation (measured edge-to-edge) from one another of about 0.2-5 μm. In embodiments, the mean or median separation is about or at least about 1.0, 1.1, 1.2, 1.3, 1.4, 1.5, 1.6, 1.7, 1.8, 1.9, or 2.0 μm. In embodiments, the mean or median separation is about 1.0, 1.1, 1.2, 1.3, 1.4, 1.5, 1.6, 1.7, 1.8, 1.9, or 2.0 μm.

In embodiments, the sequencing includes sequencing by synthesis, sequencing by ligation, sequencing-by-binding, or pyrosequencing. In embodiments, generating a first sequencing read or a second sequencing read includes a sequencing by synthesis process. In embodiments, generating a first sequencing read or a second sequencing read includes sequencing-by-binding. As used herein, “sequencing-by-binding” refers to a sequencing technique wherein specific binding of a polymerase and cognate nucleotide to a primed template nucleic acid molecule (e.g., blocked primed template nucleic acid molecule) is used for identifying the next correct nucleotide to be incorporated into the primer strand of the primed template nucleic acid molecule. The specific binding interaction need not result in chemical incorporation of the nucleotide into the primer. In some embodiments, the specific binding interaction can precede chemical incorporation of the nucleotide into the primer strand or can precede chemical incorporation of an analogous, next correct nucleotide into the primer. Thus, detection of the next correct nucleotide can take place without incorporation of the next correct nucleotide. As used herein, the “next correct nucleotide” (sometimes referred to as the “cognate” nucleotide) is the nucleotide having a base complementary to the base of the next template nucleotide. The next correct nucleotide will hybridize at the 3′-end of a primer to complement the next template nucleotide. The next correct nucleotide can be, but need not necessarily be, capable of being incorporated at the 3′ end of the primer. For example, the next correct nucleotide can be a member of a ternary complex that will complete an incorporation reaction or, alternatively, the next correct nucleotide can be a member of a stabilized ternary complex that does not catalyze an incorporation reaction. A nucleotide having a base that is not complementary to the next template base is referred to as an “incorrect” (or “non-cognate”) nucleotide.

In embodiments, the method further includes generating a sequencing read. In embodiments, generating a sequencing read includes executing a plurality of sequencing cycles, each cycle including extending the sequencing primer by incorporating a nucleotide or nucleotide analogue using a polymerase and detecting a characteristic signature indicating that the nucleotide or nucleotide analogue has been incorporated. In embodiments, the method further includes incorporating one or more unmodified dNTPs or one or more ddNTPs into the 3′ end of the extended sequencing primer.

In embodiments, generating a sequencing read includes sequencing by synthesis, sequencing-by-binding, sequencing by ligation, or pyrosequencing.

In embodiments, sequencing includes a plurality of sequencing cycles. In embodiments, sequencing includes 20 to 100 sequencing cycles. In embodiments, sequencing includes 50 to 100 sequencing cycles. In embodiments, sequencing includes 50 to 300 sequencing cycles. In embodiments, sequencing includes 50 to 150 sequencing cycles. In embodiments, sequencing includes 50 to 100 sequencing cycles. In embodiments, sequencing includes at least 10, 20, 30 40, or 50 sequencing cycles. In embodiments, sequencing includes at least 10 sequencing cycles. In embodiments, sequencing includes 10 to 20 sequencing cycles. In embodiments, sequencing includes 10, 11, 12, 13, 14, or 15 sequencing cycles. In embodiments, sequencing includes (a) extending a sequencing primer by incorporating a labeled nucleotide, or labeled nucleotide analogue and (b) detecting the label to generate a signal for each incorporated nucleotide or nucleotide analogue.

In embodiments, the methods of making and sequencing integrated strand complements provided herein include producing a plurality of sequencing reads. In embodiments, each sequencing read includes at least a portion (e.g., a first or second hybridization sequence, or a primer binding sequence) of two or more interposing oligonucleotide probes, or complements thereof. In embodiments, each sequencing read includes at least a portion (e.g., a first or second hybridization sequence, or a primer binding sequence) of three or more interposing oligonucleotide probes, or complements thereof. In embodiments, each sequencing read includes two or more interposing oligonucleotide probes, or complements thereof. In embodiments, each sequencing read includes three or more interposing oligonucleotide probes, or complements thereof. In embodiments, each sequencing read includes a portion of two or more interposing oligonucleotide probes, or complements thereof. In embodiments, each sequencing read includes a portion of two or more interposing oligonucleotide probes, or complements thereof. In embodiments, each sequencing read includes at least a portion of three interposing oligonucleotide probes, or complements thereof. In embodiments, each sequencing read includes a portion of one interposing oligonucleotide probe, or complement thereof. In embodiments, each sequencing read includes at least a portion of one interposing oligonucleotide probes, or complement thereof. In embodiments, each sequence read does not includes a portion of an interposing oligonucleotide probe, or complement thereof.

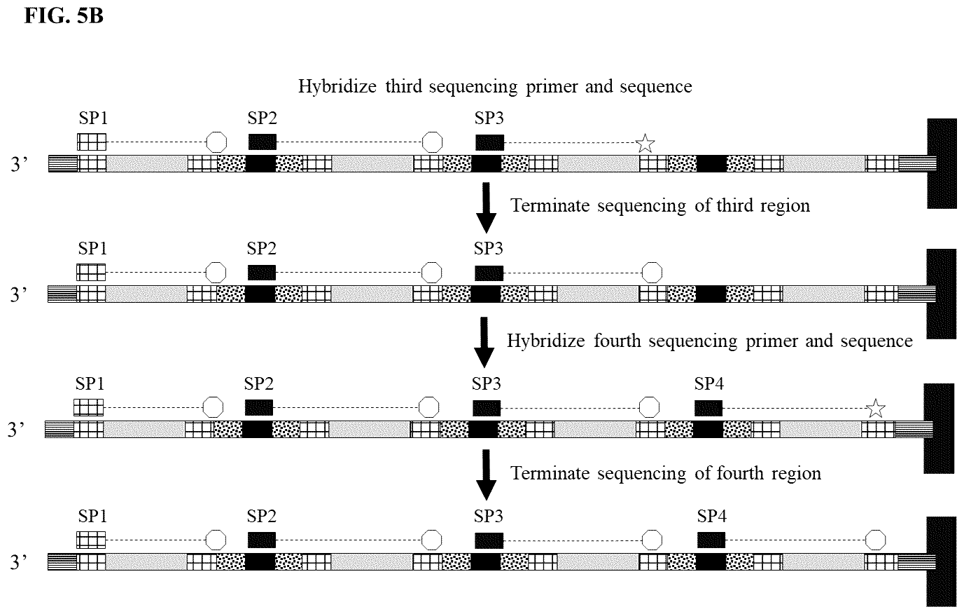

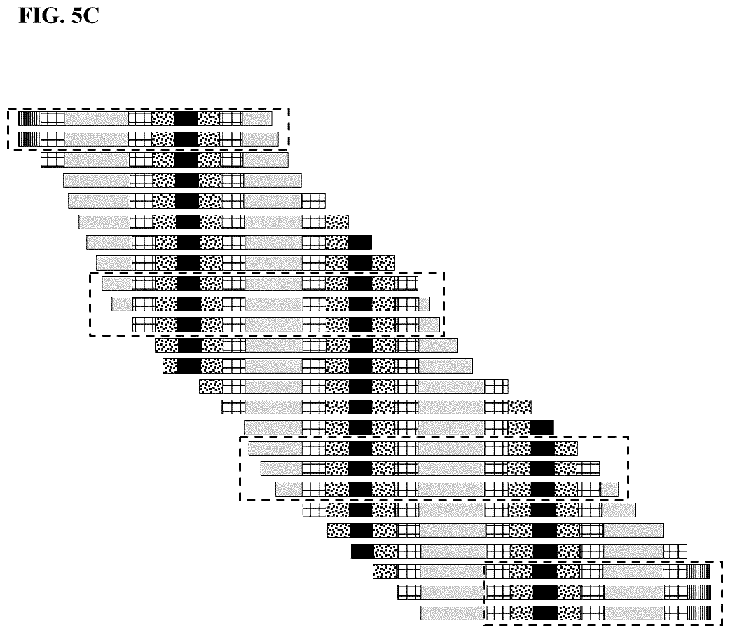

In embodiments, the method further includes sequencing the amplified product of step (d). In embodiments, the sequencing further includes (a) producing a plurality of sequencing reads; (b) grouping sequencing reads based on co-occurrence of interposing oligonucleotide probe sequences; and (c) within each group, aligning the sequencing reads that belong to the same strand of an original template nucleic acid based on the sequences of the interposing oligonucleotide probe sequences (see for example FIG. 5C). By “grouping sequencing reads based on co-occurrence of interposing oligonucleotide probe sequences” it is meant the that the sequencing reads are grouped together if, for example, each sequencing read in a group includes a sequence from the same interposing oligonucleotide probe. For example, each of the sequencing reads in a group may include the same sequencing primer binding sequence from the same interposing oligonucleotide probe (as illustrated in FIG. 5C). In embodiments, prior to sequencing, the method further includes hybridizing a sequencing primer to the primer binding sequence of the integrated oligonucleotide probe. In embodiments, prior to sequencing, the method further includes hybridizing a sequencing primer to the primer binding sequence of one of the plurality of interposing oligonucleotide probes in the integrated strands. In embodiments, prior to sequencing, the method further includes hybridizing a sequencing primer to the template nucleic acid sequence in the integrated strands.

In embodiments, the method includes sequencing the first and/or the second strand of a double-stranded amplification product by extending a sequencing primer hybridized thereto. A variety of sequencing methodologies can be used such as sequencing-by-synthesis (SBS), pyrosequencing, sequencing by ligation (SBL), or sequencing by hybridization (SBH). Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568; and. 6,274,320, each of which is incorporated herein by reference in its entirety). In pyrosequencing, released PPi can be detected by being converted to adenosine triphosphate (ATP) by ATP sulfurylase, and the level of ATP generated can be detected via light produced by luciferase. In this manner, the sequencing reaction can be monitored via a luminescence detection system. In both SBL and SBH methods, target nucleic acids, and amplicons thereof, that are present at features of an array are subjected to repeated cycles of oligonucleotide delivery and detection. SBL methods, include those described in Shendure et al. Science 309:1728-1732 (2005); U.S. Pat. Nos. 5,599,675; and 5,750,341, each of which is incorporated herein by reference in its entirety; and the SBH methodologies are as described in Bains et al., Journal of Theoretical Biology 135(3), 303-7 (1988); Drmanac et al., Nature Biotechnology 16, 54-58 (1998); Fodor et al., Science 251(4995), 767-773 (1995); and WO 1989/10977, each of which is incorporated herein by reference in its entirety.

In SBS, extension of a nucleic acid primer along a nucleic acid template is monitored to determine the sequence of nucleotides in the template. The underlying chemical process can be catalyzed by a polymerase, wherein fluorescently labeled nucleotides are added to a primer (thereby extending the primer) in a template dependent fashion such that detection of the order and type of nucleotides added to the primer can be used to determine the sequence of the template. A plurality of different nucleic acid fragments that have been attached at different locations of an array can be subjected to an SBS technique under conditions where events occurring for different templates can be distinguished due to their location in the array. In embodiments, the sequencing step includes annealing and extending a sequencing primer to incorporate a detectable label that indicates the identity of a nucleotide in the target polynucleotide, detecting the detectable label, and repeating the extending and detecting steps. In embodiments, the methods include sequencing one or more bases of a target nucleic acid by extending a sequencing primer hybridized to a target nucleic acid (e.g., an amplification product produced by the amplification methods described herein). In embodiments, the sequencing step may be accomplished by a sequencing-by-synthesis (SBS) process. In embodiments, sequencing includes a sequencing by synthesis process, where individual nucleotides are identified iteratively, as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. Nos. 10,738,072, 7,541,444 and 7,057,026. Once such a modified nucleotide has been incorporated into the growing polynucleotide chain complementary to the region of the template being sequenced, there is no free 3′-OH group available to direct further sequence extension and therefore the polymerase cannot add further nucleotides. Once the identity of the base incorporated into the growing chain has been determined, the 3′ block may be removed to allow addition of the next successive nucleotide. By ordering the products derived using these modified nucleotides it is possible to deduce the DNA sequence of the DNA template. Non-limiting examples of suitable labels are described in U.S. Pat. Nos. 8,178,360, 5,188,934 (4,7-dichlorofluorscein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthene dyes): U.S. Pat. No. 5,688,648 (energy transfer dyes); and the like.

Sequencing includes, for example, detecting a sequence of signals. Examples of sequencing include, but are not limited to, sequencing by synthesis (SBS) processes in which reversibly terminated nucleotides carrying fluorescent dyes are incorporated into a growing strand, complementary to the target strand being sequenced. In embodiments, the nucleotides are labeled with up to four unique fluorescent dyes. In embodiments, the nucleotides are labeled with at least two unique fluorescent dyes. In embodiments, the readout is accomplished by epifluorescence imaging. A variety of sequencing chemistries are available, non-limiting examples of which are described herein.

In embodiments, the methods of sequencing provided herein include aligning a portion of each sequencing read to a reference sequence. General methods for performing sequence alignments are known to those skilled in the art. Examples of suitable alignment algorithms, include but are not limited to the Needleman-Wunsch algorithm (see e.g. the EMBOSS Needle aligner available at www.ebi.ac.uk/Tools/psa/emboss_needle/, optionally with default settings), the BLAST algorithm (see e.g. the BLAST alignment tool available at blast.ncbi.nlm.nih.gov/Blast.cgi, optionally with default settings), or the Smith-Waterman algorithm (see e.g. the EMBOSS Water aligner available at www.ebi.ac.uk/Tools/psa/emboss_water/, optionally with default settings). Optimal alignment may be assessed using any suitable parameters of a chosen algorithm, including default parameters. In embodiments, the reference sequence is a reference genome. In embodiments, the methods of sequencing a template nucleic acid further include generating overlapping sequence reads and assembling them into a contiguous nucleotide sequence of a nucleic acid of interest. Assembly algorithms known in the art can align and merge overlapping sequence reads generated by methods of several embodiments herein to provide a contiguous sequence of a nucleic acid of interest. A person of ordinary skill in the art will understand which sequence assembly algorithms or sequence assemblers are suitable for a particular purpose taking into account the type and complexity of the nucleic acid of interest to be sequenced (e.g. genomic, PCR product, or plasmid), the number and/or length of deletion products or other overlapping regions generated, the type of sequencing methodology performed, the read lengths generated, whether assembly is de novo assembly of a previously unknown sequence or mapping assembly against a backbone sequence, etc. Furthermore, an appropriate data analysis tool will be selected based on the function desired, such as alignment of sequence reads, base-calling and/or polymorphism detection, de novo assembly, assembly from paired or unpaired reads, and genome browsing and annotation. In several embodiments, overlapping sequence reads can be assembled by sequence assemblers, including but not limited to ABySS, AMOS, Arachne WGA, CAP3, PCAP, Celera WGA Assembler/CABOG, CLC Genomics Workbench, CodonCode Aligner, Euler, Euler-sr, Forge, Geneious, MIRA, miraEST, NextGENe, Newbler, Phrap, TIGR Assembler, Sequencher, SeqMan NGen, SHARCGS, SSAKE, Staden gap4 package, VCAKE, Phusion assembler, Quality Value Guided SRA (QSRA), Velvet (algorithm), and the like. It will be understood that overlapping sequence reads can also be assembled into contigs or the full contiguous sequence of the nucleic acid of interest by available means of sequence alignment, computationally or manually, whether by pairwise alignment or multiple sequence alignment of overlapping sequence reads. Algorithms suited for short-read sequence data may be used in a variety of embodiments, including but not limited to Cross_match, ELAND, Exonerate, MAQ, Mosaik, RMAP, SHRiMP, SOAP, SSAHA2, SXOligoSearch, ALLPATHS, Edena, Euler-SR, SHARCGS, SHRAP, SSAKE, VCAKE, Velvet, PyroBayes, PbShort, and ssahaSNP. In embodiments, aligning to a reference sequence is useful to validate the approaches described herein.

In embodiments, the methods of sequencing provided herein further include forming a consensus sequence for reads having the same primer binding sequence, template nucleic acid sequence, or a portion thereof. In embodiments, forming a consensus sequence does not include comparing barcode sequences (e.g., UMI sequences). In embodiments, the consensus sequence is obtained by comparing all sequencing reads aligning at a given nucleotide position (optionally, only among those reads identified as originating from the same template nucleic acid molecule), and identifying the nucleotide at that position as the one shared by a majority of the aligned reads.

A variety of suitable sequencing platforms are available for implementing methods disclosed herein (e.g., for performing the sequencing reaction). Non-limiting examples include SMRT (single-molecule real-time sequencing), ion semiconductor, pyrosequencing, sequencing by synthesis, combinatorial probe anchor synthesis, SOLiD sequencing (sequencing by ligation), and nanopore sequencing. Sequencing platforms include those provided by Singular Genomics™ (e.g., the G4™ system), Illumina™, Inc. (e.g., HiSeq™, MiSeq™, NextSeq™, or NovaSeq™ systems), Life Technologies™ (e.g., ABI PRISM™, or SOLiD™ systems), Pacific Biosciences (e.g., systems using SMRT™ Technology such as the Sequel™ or RS II™ systems), or Qiagen (e.g., Genereader™ system). See, for example U.S. Pat. Nos. 7,211,390; 7,244,559; 7,264,929; 6,255,475; 6,013,445; 8,882,980; 6,664,079; and 9,416,409.

In embodiments, the methods of sequencing described herein further include computationally reconstructing sequences of a plurality of individual strands of original sample polynucleotides by removing primer binding site-derived sequences and joining sequences for adjacent portions of the sample polynucleotide. Reconstruction can be performed on individual reads, or on consensus sequences produced from those reads.

In embodiments, the methods of sequencing described herein further include aligning computationally reconstructed sequences.

Flow cells provide a convenient format for housing an array of clusters produced by the methods described herein, in particular when subjected to an SBS or other detection technique that involves repeated delivery of reagents in cycles. For example, to initiate a first SBS cycle, one or more labeled nucleotides and a DNA polymerase in a buffer, can be flowed into/through a flow cell that houses an array of clusters. The clusters of an array where primer extension causes a labeled nucleotide to be incorporated can then be detected. Optionally, the nucleotides can further include a reversible termination moiety that temporarily halts further primer extension once a nucleotide has been added to a primer. For example, a nucleotide analog having a reversible terminator moiety can be added to a primer such that subsequent extension cannot occur until a deblocking agent (e.g., a reducing agent) is delivered to remove the moiety. Thus, for embodiments that use reversible termination, a deblocking reagent (e.g., a reducing agent) can be delivered to the flow cell (before, during, or after detection occurs). Washes can be carried out between the various delivery steps as needed. The cycle can then be repeated N times to extend the primer by N nucleotides, thereby detecting a sequence of length N. Example SBS procedures, fluidic systems and detection platforms that can be readily adapted for use with an array produced by the methods of the present disclosure are described, for example, in Bentley et al., Nature 456:53-59 (2008), US Patent Publication 2018/0274024, WO 2017/205336, US Patent Publication 2018/0258472, each of which are incorporated herein in their entirety for all purposes.

Use of the sequencing method outlined above is a non-limiting example, as essentially any sequencing methodology which relies on successive incorporation of nucleotides into a polynucleotide chain can be used. Suitable alternative techniques include, for example, pyrosequencing methods, FISSEQ (fluorescent in situ sequencing), MPSS (massively parallel signature sequencing), or sequencing by ligation-based methods.

In embodiments, generating a sequencing read includes determining the identity of the nucleotides in the template polynucleotide (or complement thereof). In embodiments, a sequencing read, e.g., a first sequencing read or a second sequencing read, includes determining the identity of a portion (e.g., 1, 2, 5, 10, 20, 50 nucleotides) of the total template polynucleotide. In embodiments the first sequencing read determines the identity of 5-10 nucleotides and the second sequencing read determines the identity of more than 5-10 nucleotides (e.g., 11 to 200 nucleotides). In embodiments the first sequencing read determines the identity of more than 5-10 nucleotides (e.g., 11 to 200 nucleotides) and the second sequencing read determines the identity of 5-10 nucleotides. In embodiments, following the generation of a sequencing read, subsequent extension is performed using a plurality of standard (e.g., non-modified) dNTPs until the complementary strand is copied. In other embodiments, following the generation of a sequencing read, subsequent extension is performed using a plurality of dideoxy nucleotide triphosphates (ddNTPs) to prevent further extension of the first sequencing read product during a second sequencing read. In embodiments, following the identification of at least 5-10 (e.g., 11 to 200 nucleotides, or up to 1000 nucleotides), subsequent extension is performed using a plurality of standard (e.g., non-modified) dNTPs until the complementary strand is copied. In embodiments, following the identification of at least 5-10 (e.g., 11 to 200 nucleotides, or up to 1000 nucleotides), subsequent extension is performed using a plurality of dideoxy nucleotide triphosphates (ddNTPs) to prevent further extension of the sequencing read product.

In embodiments, the sequencing method relies on the use of modified nucleotides that can act as reversible reaction terminators. Once the modified nucleotide has been incorporated into the growing polynucleotide chain complementary to the region of the template being sequenced there is no free 3′-OH group available to direct further sequence extension and therefore the polymerase cannot add further nucleotides. Once the identity of the base incorporated into the growing chain has been determined, the 3′ reversible terminator may be removed to allow addition of the next successive nucleotide. These such reactions can be done in a single experiment if each of the modified nucleotides has attached a different label, known to correspond to the particular nucleobase, to facilitate discrimination between the bases added at each incorporation step. Alternatively, a separate reaction may be carried out containing each of the modified nucleotides separately.

The modified nucleotides may carry a label (e.g., a fluorescent label) to facilitate their detection. Each nucleotide type may carry a different fluorescent label. However, the detectable label need not be a fluorescent label. Any label can be used which allows the detection of an incorporated nucleotide. One method for detecting fluorescently labeled nucleotides includes using laser light of a wavelength specific for the labeled nucleotides, or the use of other suitable sources of illumination. The fluorescence from the label on the nucleotide may be detected (e.g., by a CCD camera or other suitable detection means).

In embodiments, the methods of sequencing a nucleic acid include extending a complementary polynucleotide (e.g., a primer) that is hybridized to the nucleic acid by incorporating a first nucleotide. In embodiments, the method includes a buffer exchange or wash step. In embodiments, the methods of sequencing a nucleic acid include a sequencing solution. The sequencing solution includes (a) an adenine nucleotide, or analog thereof, (b) (i) a thymine nucleotide, or analog thereof, or (ii) a uracil nucleotide, or analog thereof; (c) a cytosine nucleotide, or analog thereof, and (d) a guanine nucleotide, or analog thereof.

In embodiments, the sequenced nucleotides include a scar remnant (e.g., an alkynyl moiety attached to the nucleobase). In embodiments, the nucleotides have the formula:

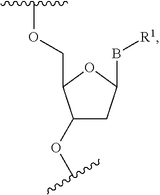

wherein B is a nucleobase, R¹ is the scar remnant, and “ [symbol] ” is the attachment point to the remainder of the sequenced strand polynucleotide.

In embodiments, B is a divalent nucleobase. In embodiments, B is

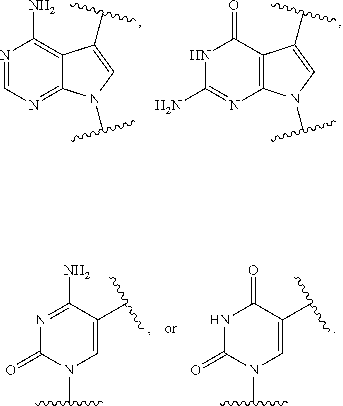 [SMILES: *c1cn(*)c2ncnc(N)c12 ; *c1cn(*)c2nc(N)[nH]c(=O)c12 ; *c1cn(*)c(=O)nc1N ; *c1cn(*)c(=O)[nH]c1=O]

In embodiments, B is

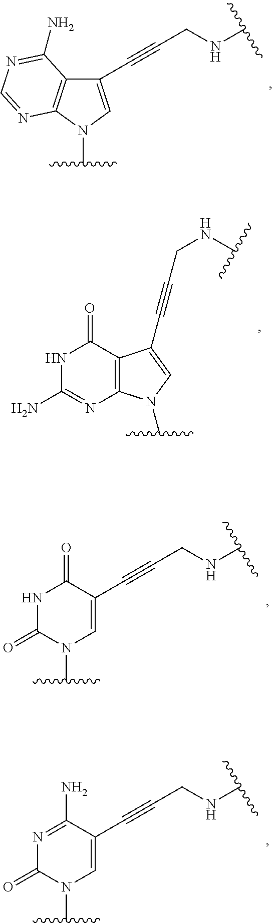 [SMILES: *NCC#Cc1cn(*)c2ncnc(N)c12 ; *NCC#Cc1cn(*)c2nc(N)[nH]c(=O)c12 ; *NCC#Cc1cn(*)c(=O)[nH]c1=O ; *NCC#Cc1cn(*)c(=O)nc1N]

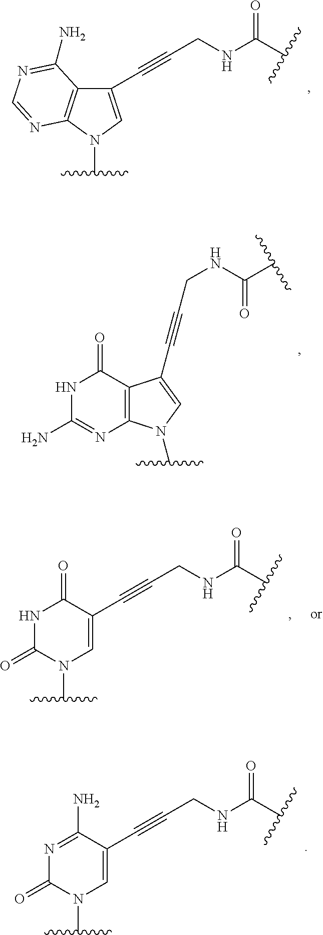 [SMILES: *C(=O)NCC#Cc1cn(*)c2ncnc(N)c12 ; *C(=O)NCC#Cc1cn(*)c2nc(N)[nH]c(=O)c12 ; *C(=O)NCC#Cc1cn(*)c(=O)[nH]c1=O ; *C(=O)NCC#Cc1cn(*)c(=O)nc1N]

In embodiments, R¹ is hydrogen, —OH, —NH, a substituted or unsubstituted alkyl or substituted or unsubstituted heteroalkyl. In embodiments, R¹ is hydrogen. In embodiments, R¹ is —OH. In embodiments, R¹ is —NH. In embodiments, R¹ is a substituted or unsubstituted alkyl or substituted or unsubstituted heteroalkyl. In embodiments, R¹ is a substituted or unsubstituted alkenyl. In embodiments, R¹ is a substituted or unsubstituted alkynyl. In embodiments, R¹ is a substituted or unsubstituted heteroalkenyl. In embodiments, R¹ is a substituted or unsubstituted heteroalkynyl. In embodiments, R¹ is a substituted (e.g., substituted with a substituent group, size-limited substituent group, or lower substituent group) or unsubstituted alkyl or substituted (e.g., substituted with a substituent group, size-limited substituent group, or lower substituent group) or unsubstituted heteroalkyl. In embodiments, R¹ is substituted with an oxo or —OH.

In embodiments, R¹ is an oxo-substituted heteroalkyl (e.g., 2 to 10 membered heteroalkyl, 2 to 8 membered heteroalkyl, or 4 to 8 membered heteroalkyl). In embodiments, R¹ is an oxo-substituted heteroalkenyl (e.g., 2 to 10 membered heteroalkenyl, 2 to 8 membered heteroalkenyl, or 4 to 8 membered heteroalkenyl). In embodiments, R¹ is an oxo-substituted heteroalkynyl (e.g., 2 to 10 membered heteroalkynyl, 2 to 8 membered heteroalkynyl, or 4 to 8 membered heteroalkynyl). In embodiments, R¹ is an oxo-substituted 10 membered heteroalkynyl. In embodiments, R¹ is an oxo-substituted 9 membered heteroalkynyl. In embodiments, R¹ is an oxo-substituted 8 membered heteroalkynyl. In embodiments, R¹ is an oxo-substituted 7 membered heteroalkynyl. In embodiments, R¹ is an oxo-substituted 6 membered heteroalkynyl.

In embodiments, the one or more nucleotides including a scar remnant include a nucleobase having the formula,

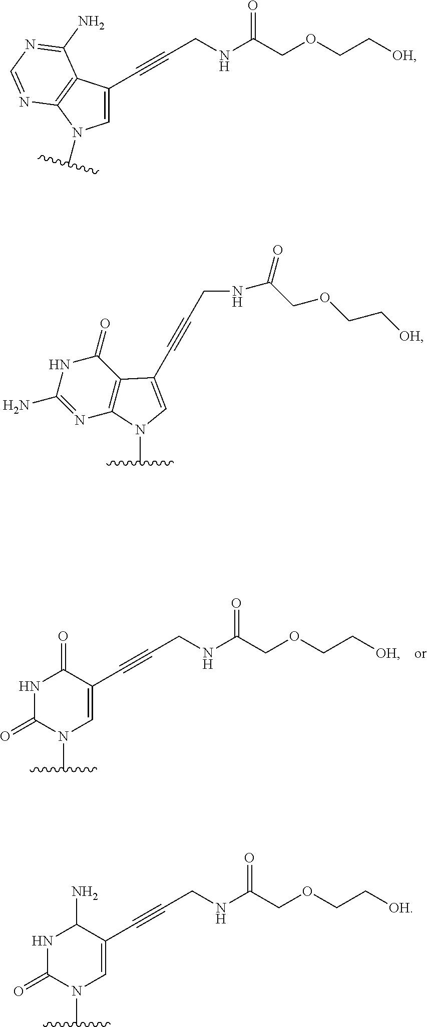 [SMILES: *n1cc(C#CCNC(=O)COCCO)c2c(N)ncnc21 ; *n1cc(C#CCNC(=O)COCCO)c2c(=O)[nH]c(N)nc21 ; *n1cc(C#CCNC(=O)COCCO)c(=O)[nH]c1=O ; *N1C=C(C#CCNC(=O)COCCO)C(N)NC1=O]

In embodiments, the one or more nucleotides including a scar remnant include a nucleobase having the formula

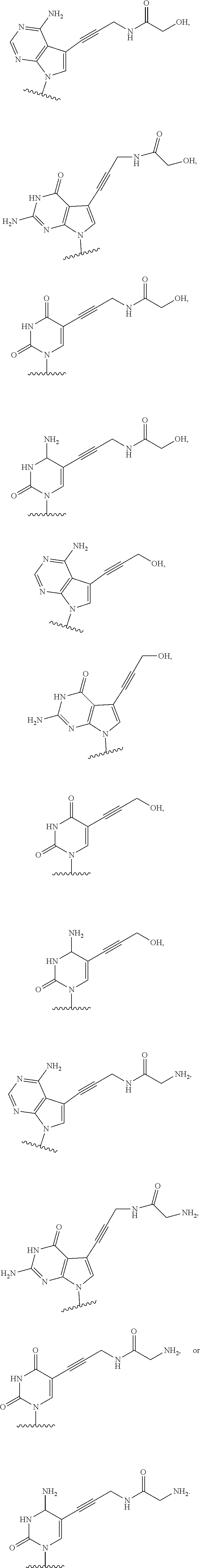

In an aspect is provided method of sequencing a polynucleotide, the method including: hybridizing a first sequencing primer to a first sequence of the polynucleotide and incorporating with a polymerase one or more nucleotides into the first sequencing primer to create a first extension strand, and detecting the one or more incorporated nucleotides so as to identify each incorporated nucleotide in the first extension strand; contacting the first extension strand with a blocking element thereby terminating extension of the first extension strand thereby forming a blocked first extension strand; followed by hybridizing a second sequencing primer to a second sequence of the polynucleotide and incorporating with a polymerase one or more nucleotides into the second sequencing primer to create a second extension strand, and detecting the one or more incorporated nucleotides so as to identify each incorporated nucleotide in the second extension strand; contacting the second extension strand with a blocking element thereby terminating extension of the second extension strand thereby forming a blocked second extension strand; followed by hybridizing a third sequencing primer to a third sequence of the polynucleotide and incorporating with a polymerase one or more nucleotides into the third sequencing primer to create a third extension strand, and detecting the one or more incorporated nucleotides so as to identify each incorporated nucleotide in the third extension strand.

### III. Compositions & Kits

In an aspect is provided a kit including: (a) a plurality of interposing oligonucleotide probes capable of hybridizing to a template nucleic acid, the interposing oligonucleotide probes including from 5′ to 3′: i. a first hybridization sequence complementary to a first sequence of the template nucleic acid; ii. a loop region including a primer binding sequence; and iii. a second hybridization sequence complementary to a second sequence of the template nucleic acid; (b) a plurality of 5′ terminal oligonucleotide probes capable of hybridizing to a template nucleic acid, the 5′ terminal oligonucleotide probes including from 5′ to 3′: i. a hybridization sequence complementary to a 5′ terminal sequence of the template nucleic acid, wherein the 5′ terminal sequence is upstream of the template nucleic acid sequence complementary to the interposing oligonucleotide probes (see, e.g., FIG. 4 for an illustration of the hybridized 5′ terminal oligonucleotide probe); and ii. a primer binding sequence; and (c) a plurality of 3′ terminal oligonucleotide probes capable of hybridizing to a template nucleic acid, the 3′ terminal oligonucleotide probes including from 3′ to 5′: i. a hybridization sequence complementary to a 3′ terminal sequence of the template nucleic acid, wherein the 3′ terminal sequence is downstream of the template nucleic acid sequence complementary to the interposing oligonucleotide probes (see, e.g., FIG. 4 for an illustration of the hybridized 3′ terminal oligonucleotide probe); and ii. a primer binding sequence. In embodiments, the 5′ terminal oligonucleotide probes capable of hybridizing to a template nucleic acid, from 5′ to 3′, include: i. a hybridization sequence complementary to a 5′ terminal sequence of the template nucleic acid, wherein the 5′ terminal sequence is downstream of the template nucleic acid sequence complementary to the interposing oligonucleotide probes. In embodiments, the 3′ terminal oligonucleotide probes capable of hybridizing to a template nucleic acid, the 3′ terminal oligonucleotide probes including from 3′ to 5′: i. a hybridization sequence complementary to a 3′ terminal sequence of the template nucleic acid, wherein the 3′ terminal sequence is upstream of the template nucleic acid sequence complementary to the interposing oligonucleotide probes.

In embodiments, the plurality of 5′ terminal oligonucleotide probes include from 5′ to 3′: i. a first hybridization sequence complementary to a first 5′ terminal sequence of the template nucleic acid; ii. a loop region including a primer binding sequence; and iii. a second hybridization sequence complementary to a second 5′ terminal sequence of the template nucleic acid, wherein the first and second 5′ terminal sequences are upstream of the template nucleic acid sequence complementary to the interposing oligonucleotide probes. In embodiments, the 5′ terminal oligonucleotide probes include from 5′ to 3′: i. a first hybridization sequence complementary to a first 5′ terminal sequence of the template nucleic acid; ii. a loop region including a primer binding sequence; and iii. a second hybridization sequence complementary to a second 5′ terminal sequence of the template nucleic acid, wherein the first and second 5′ terminal sequences are downstream of the template nucleic acid sequence complementary to the interposing oligonucleotide probes.

In embodiments, the plurality of 3′ terminal oligonucleotide probes include from 3′ to 5′: i. a first hybridization sequence complementary to a first 3′ terminal sequence of the template nucleic acid; ii. a loop region including a primer binding sequence; and iii. a second hybridization sequence complementary to a second 3′ terminal sequence of the template nucleic acid, wherein the first and second 3′ terminal sequences are downstream of the template nucleic acid sequence complementary to the interposing oligonucleotide probes. In embodiments, the plurality of 3′ terminal oligonucleotide probes include from 3′ to 5′: i. a first hybridization sequence complementary to a first 3′ terminal sequence of the template nucleic acid; ii. a loop region including a primer binding sequence; and iii. a second hybridization sequence complementary to a second 3′ terminal sequence of the template nucleic acid, wherein the first and second 3′ terminal sequences are upstream of the template nucleic acid sequence complementary to the interposing oligonucleotide probes.

In an aspect is provided a kit. Generally, the kit includes one or more containers providing a composition and one or more additional reagents (e.g., a buffer suitable for polynucleotide extension). The kit may also include a template nucleic acid (DNA and/or RNA), one or more primer polynucleotides, nucleoside triphosphates (including, e.g., deoxyribonucleotides, ribonucleotides, labeled nucleotides, and/or modified nucleotides), buffers, salts, and/or labels (e.g., fluorophores).

In embodiments, the kit includes a sequencing polymerase, and one or more amplification polymerases. In embodiments, the sequencing polymerase is capable of incorporating modified nucleotides. In embodiments, the polymerase is a DNA polymerase. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ι DNA polymerase, Pol κ DNA polymerase, Pol ζ DNA polymerase, Pol γ DNA polymerase, Pol θ DNA polymerase, Pol ν DNA polymerase, or a thermophilic nucleic acid polymerase (e.g., Therminator γ, 9° N polymerase (exo-), Therminator II, Therminator III, or Therminator IX). In embodiments, the DNA polymerase is a thermophilic nucleic acid polymerase. In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044, each of which are incorporated herein by reference for all purposes). In embodiments, the kit includes a strand-displacing polymerase. In embodiments, the kit includes a strand-displacing polymerase, such as a phi29 polymerase, phi29 mutant polymerase or a thermostable phi29 mutant polymerase.

In embodiments, the kit includes a buffered solution. Typically, the buffered solutions contemplated herein are made from a weak acid and its conjugate base or a weak base and its conjugate acid. For example, sodium acetate and acetic acid are buffer agents that can be used to form an acetate buffer. Other examples of buffer agents that can be used to make buffered solutions include, but are not limited to, Tris, bicine, tricine, HEPES, TES, MOPS, MOPSO and PIPES. Additionally, other buffer agents that can be used in enzyme reactions, hybridization reactions, and detection reactions are known in the art. In embodiments, the buffered solution can include Tris. With respect to the embodiments described herein, the pH of the buffered solution can be modulated to permit any of the described reactions. In some embodiments, the buffered solution can have a pH greater than pH 7.0, greater than pH 7.5, greater than pH 8.0, greater than pH 8.5, greater than pH 9.0, greater than pH 9.5, greater than pH 10, greater than pH 10.5, greater than pH 11.0, or greater than pH 11.5. In other embodiments, the buffered solution can have a pH ranging, for example, from about pH 6 to about pH 9, from about pH 8 to about pH 10, or from about pH 7 to about pH 9. In embodiments, the buffered solution can include one or more divalent cations. Examples of divalent cations can include, but are not limited to, Mg²⁺, Mn²⁺, Zn²⁺, and Ca²⁺. In embodiments, the buffered solution can contain one or more divalent cations at a concentration sufficient to permit hybridization of a nucleic acid. The kit may also include a flow cell. In embodiments, kit includes the solid support and a flow cell carrier (e.g., a flow cell carrier as described in US 2021/0190668, which is incorporated herein by reference for all purposes).

In embodiments, the kit includes components useful for ligating polynucleotides using a ligation enzyme (e.g., CircLigase™ enzyme, Taq DNA Ligase, HiFi Taq DNA Ligase, T4 DNA ligase, T4 RNA ligase, T4 RNA ligase 2, or Ampligase® DNA Ligase). For example, such a kit further includes the following components: (a) reaction buffer for controlling pH and providing an optimized salt composition for a ligation enzyme (e.g., CircLigase™ enzyme, Taq DNA Ligase, HiFi Taq DNA Ligase, T4 DNA ligase, T4 RNA ligase 2, or Ampligase® DNA Ligase), and (b) ligation enzyme cofactors, such as ATP and a divalent ion (e.g., Mn²⁺ or Mg²⁺).

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay, etc.) from one location to another. For example, kits include one or more enclosures (e.g., boxes) containing the relevant reaction reagents and/or supporting materials. As used herein, the term “fragmented kit” refers to a delivery system including two or more separate containers that each contain a subportion of the total kit components. The containers may be delivered to the intended recipient together or separately. For example, a first container may contain an enzyme for use in an assay, while a second container contains oligonucleotides. In contrast, a “combined kit” refers to a delivery system containing all of the components of a reaction assay in a single container (e.g., in a single box housing each of the desired components). The term “kit” includes both fragmented and combined kits. In embodiments, the kit includes, without limitation, nucleic acid primers, probes, adapters, enzymes, and the like, and are each packaged in a container, such as, without limitation, a vial, tube or bottle, in a package suitable for commercial distribution, such as, without limitation, a box, a sealed pouch, a blister pack and a carton. The package typically contains a label or packaging insert indicating the uses of the packaged materials. As used herein, “packaging materials” includes any article used in the packaging for distribution of reagents in a kit, including without limitation containers, vials, tubes, bottles, pouches, blister packaging, labels, tags, instruction sheets and package inserts.

Adapters and/or primers may be supplied in the kits ready for use, as concentrates-requiring dilution before use, or in a lyophilized or dried form requiring reconstitution prior to use. If required, the kits may further include a supply of a suitable diluent for dilution or reconstitution of the primers and/or adapters. Optionally, the kits may further include supplies of reagents, buffers, enzymes, and dNTPs for use in carrying out nucleic acid amplification and/or sequencing. Further components which may optionally be supplied in the kit include sequencing primers suitable for sequencing templates prepared using the methods described herein.

In embodiments, provided herein is a composition including a template nucleic acid hybridized to a plurality of oligonucleotides probes (e.g., interposing probes) according to any of the aspects of interposing probes described herein. In embodiments the template nucleic acid is an RNA transcript. In embodiments, the polynucleotide is mRNA.

In embodiments, provided herein is a composition including a template nucleic acid hybridized to a plurality of oligonucleotides probes (e.g., interposing probes) according to any of the aspects of interposing probes described herein, where the second hybridization sequence is at least twice as long as the first hybridization sequence (e.g., the first hybridization sequence is 5 nucleotides in length and the second is at least 10 nucleotides in length). In embodiments, the second hybridization sequence is at least three times as long as the first hybridization sequence. In embodiments, the second hybridization sequence is at least four times as long as the first hybridization sequence. In embodiments, the second hybridization sequence is more than four times as long as the first hybridization sequence. In embodiments, the second hybridization sequence is the same length as the first hybridization sequence. In embodiments, the template nucleic acid can include any nucleic acid of interest. The nucleic acid can include DNA, RNA, peptide nucleic acid (PNA), morpholino nucleic acid, locked nucleic acid (LNA), glycol nucleic acid, threose nucleic acid, mixtures thereof, and hybrids thereof. In embodiments, the nucleic acid is obtained from one or more source organisms. In some embodiments, the nucleic acid can include a selected sequence or a portion of a larger sequence. In embodiments, sequencing a portion of a nucleic acid or a fragment thereof can be used to identify the source of the nucleic acid. With reference to nucleic acids, polynucleotides and/or nucleotide sequences a “portion,” “fragment” or “region” can be at least 5 consecutive nucleotides, at least 10 consecutive nucleotides, at least 15 consecutive nucleotides, at least 20 consecutive nucleotides, at least 25 consecutive nucleotides, at least 50 consecutive nucleotides, at least 100 consecutive nucleotides, or at least 150 consecutive nucleotides.

In embodiments, the template nucleic acid is at least 1000 bases (1 kb), at least 2 kb, at least 4 kb, at least 6 kb, at least 10 kb, at least 20 kb, at least 30 kb, at least 40 kb, or at least 50 kb in length. In embodiments, the entire sequence of the template nucleic acid is about 1 to 3 kb, and only a portion of that the template nucleic acid (e.g., 50 to 100 nucleotides) is sequenced at a time. In embodiments, the template nucleic acid is about 2 to 3 kb. In embodiments, the template nucleic acid is about 1 to 10 kb. In embodiments, the template nucleic acid is about 3 to 10 kb. In embodiments, the template nucleic acid is about 5 to 10 kb. In embodiments, the template nucleic acid is about 1 to 3 kb. In embodiments, the template nucleic acid is about 1 to 2 kb. In embodiments, the template nucleic acid is greater than 1 kb. In embodiments, the template nucleic acid is greater than 500 bases. In embodiments, the template nucleic acid is about 1 kb. In embodiments, the template nucleic acid is about 2 kb. In embodiments, the template nucleic acid is less than 1 kb. In embodiments, the template nucleic acid is about 500 nucleotides. In embodiments, the template nucleic acid is about 510 nucleotides. In embodiments, the template nucleic acid is about 520 nucleotides. In embodiments, the template nucleic acid is about 530 nucleotides. In embodiments, the template nucleic acid is about 540 nucleotides. In embodiments, the template nucleic acid is about 550 nucleotides. In embodiments, the template nucleic acid is about 560 nucleotides. In embodiments, the template nucleic acid is about 570 nucleotides. In embodiments, the template nucleic acid is about 580 nucleotides. In embodiments, the template nucleic acid is about 590 nucleotides. In embodiments, the template nucleic acid is about 600 nucleotides. In embodiments, the template nucleic acid is about 610 nucleotides. In embodiments, the template nucleic acid is about 620 nucleotides. In embodiments, the template nucleic acid is about 630 nucleotides. In embodiments, the template nucleic acid is about 640 nucleotides. In embodiments, the template nucleic acid is about 650 nucleotides. In embodiments, the template nucleic acid is about 660 nucleotides. In embodiments, the template nucleic acid is about 670 nucleotides. In embodiments, the template nucleic acid is about 680 nucleotides. In embodiments, the template nucleic acid is about 690 nucleotides. In embodiments, the template nucleic acid is about 700 nucleotides. In embodiments, the template nucleic acid is about 1,600 nucleotides. In embodiments, the template nucleic acid is about 1,610 nucleotides. In embodiments, the template nucleic acid is about 1,620 nucleotides. In embodiments, the template nucleic acid is about 1,630 nucleotides. In embodiments, the template nucleic acid is about 1,640 nucleotides. In embodiments, the template nucleic acid is about 1,650 nucleotides. In embodiments, the template nucleic acid is about 1,660 nucleotides. In embodiments, the template nucleic acid is about 1,670 nucleotides. In embodiments, the template nucleic acid is about 1,680 nucleotides. In embodiments, the template nucleic acid is about 1,690 nucleotides. In embodiments, the template nucleic acid is about 1,700 nucleotides. In embodiments, the template nucleic acid is about 1,710 nucleotides. In embodiments, the template nucleic acid is about 1,720 nucleotides. In embodiments, the template nucleic acid is about 1,730 nucleotides. In embodiments, the template nucleic acid is about 1,740 nucleotides. In embodiments, the template nucleic acid is about 1,750 nucleotides. In embodiments, the template nucleic acid is about 1,760 nucleotides. In embodiments, the template nucleic acid is about 1,770 nucleotides. In embodiments, the template nucleic acid is about 1,780 nucleotides. In embodiments, the template nucleic acid is about 1,790 nucleotides. In embodiments, the template nucleic acid is about 1,800 nucleotides.

In embodiments, the template nucleic acid is a nucleic acid sequence. In embodiments the template nucleic acid is an RNA transcript. RNA transcripts are responsible for the process of converting DNA into an organism's phenotype, thus by determining the types and quantity of RNA present in a sample (e.g., a cell), it is possible to assign a phenotype to the cell. RNA transcripts include coding RNA and non-coding RNA molecules, such as messenger RNA (mRNA), transfer RNA (tRNA), micro RNA (miRNA), small interfering RNA (siRNA), small nucleolar RNA (snoRNA), small nuclear RNA (snRNA), Piwi-interacting RNA (piRNA), enhancer RNA (eRNA), or ribosomal RNA (rRNA). In embodiments, the target is pre-mRNA. In embodiments, the target is heterogeneous nuclear RNA (hnRNA). In embodiments the template nucleic acid is a single stranded RNA nucleic acid sequence. In embodiments, the template nucleic acid is an RNA nucleic acid sequence or a DNA nucleic acid sequence (e.g., cDNA). In embodiments, the template nucleic acid is a cDNA target nucleic acid sequence. In embodiments, the template nucleic acid is genomic DNA (gDNA), mitochondrial DNA, chloroplast DNA, episomal DNA, viral DNA, or complementary DNA (cDNA). In embodiments, the template nucleic acid is coding RNA such as messenger RNA (mRNA), and non-coding RNA (ncRNA) such as transfer RNA (tRNA), microRNA (miRNA), small nuclear RNA (snRNA), or ribosomal RNA (rRNA).

In embodiments, the template nucleic acid is a cancer-associated gene or fragment thereof. In general, “cancer associated genes” are genes for which change in expression, change in activity of an encoded protein, mutation, or a combination of these is correlated with the occurrence of cancer. A variety of cancer-associated genes are known. In embodiments, the cancer-associated gene is a MDC, NME-2, KGF, P1GF, Flt-3L, HGF, MCP1, SAT-1, MIP-1-b, GCLM, OPG, TNF RII, VEGF-D, ITAC, MMP-10, GPI, PPP2R4, AKR1B1, Amy1A, P-Cadherin, or EPO gene or fragment thereof. In embodiments, the cancer-associated gene is a AKT1, AKT2, AKT3, ALK, AR, ARAF, ARID1A, ATM, ATR, ATRX, AXL, BAP1, BRAF, BRCA1, BRCA2, BTK, CBL, CCND1, CCND2, CCND3, CCNE1, CDK12, CDK2, CDK4, CDK6, CDKN1B, CDKN2A, CDKN2B, CHEK1, CHEK2, CREBBP, CSF1R, CTNNB1, DDR2, EGFR, ERBB2, ERBB3, ERBB4, ERCC2, ERG, ESR1, ETV1, ETV4, ETV5, EZH2, FANCA, FANCD2, FANCI, FBXW7, FGF19, FGF3, FGFR1, FGFR2, FGFR3, FGFR4, FGR, FLT3, FOXL2, GATA2, GNA11, GNAQ, GNAS, H3F3A, HIST1H3B, HNF1A, HRAS, IDH1, IDH2, IGF1R, JAK1, JAK2, JAK3, KDR, KIT, KNSTRN, KRAS, MAGOH, MAP2K1, MAP2K2, MAP2K4, MAPK1, MAX, MDM2, MDM4, MED12, MET, MLH1, MRE11A, MSH2, MSH6, MTOR, MYB, MYBL1, MYC, MYCL, MYCN, MYD88, NBN, NF1, NF2, NFE2L2, NOTCH1, NOTCH2, NOTCH3, NOTCH4, NRAS, NRG1, NTRK1, NTRK2, NTRK3, NUTM1, PALB2, PDGFRA, PDGFRB, PIK3CA, PIK3CB, PIK3R1, PMS2, POLE, PPARG, PPP2R1A, PRKACA, PRKACB, PTCH1, PTEN, PTPN11, RAC1, RAD50, RAD51, RAD51B, RAD51C, RAD51D, RAF1, RB1, RELA, RET, RHEB, RHOA, RICTOR, RNF43, ROS1, RSPO2, RSPO3, SETD2, SF3B1, SLX4, SMAD4, SMARCA4, SMARCB1, SMO, SPOP, SRC, STAT3, STK11, TERT, TOP1, TP53, TSC1, TSC2, U2AF1, or XPO1 gene, or fragment thereof. In embodiments, the cancer-associated gene is a ABL1, AKT1, ALK, APC, ATM, BRAF, CDH1, CDKN2A, CSF1R, CTNNB1, EGFR, ERBB2, ERBB4, EZH2, FBXW7, FGFR1, FGFR2, FGFR3, FLT3, GNA11, GNAQ, GNAS, HNF1A, HRAS, IDH1, IDH2, JAK2, JAK3, KDR, KIT, KRAS, MET, MLH1, MPL, NOTCH1, NPM1, NRAS, PDGFRA, PIK3CA, PTEN, PTPN11, RB1, RET, SMAD4, SMARCB1, SMO, SRC, STK11, TP53, or VHL gene, or fragment thereof.

In embodiments, the template nucleic acids are RNA nucleic acid sequences or DNA nucleic acid sequences. In embodiments, the template nucleic acids are RNA nucleic acid sequences or DNA nucleic acid sequences from the same cell. In embodiments, the template nucleic acids are RNA nucleic acid sequences. In embodiments, the RNA nucleic acid sequence is stabilized using known techniques in the art. For example, RNA degradation by RNase should be minimized using commercially available solutions (e.g., RNA Later®, RNA Protect®, or DNA/RNA Shield®). In embodiments, the template nucleic acids are messenger RNA (mRNA), transfer RNA (tRNA), micro RNA (miRNA), small interfering RNA (siRNA), small nucleolar RNA (snoRNA), small nuclear RNA (snRNA), Piwi-interacting RNA (piRNA), enhancer RNA (eRNA), or ribosomal RNA (rRNA). In embodiments, the template nucleic acid is pre-mRNA. In embodiments, the template nucleic acid is heterogeneous nuclear RNA (hnRNA). In embodiments, the template nucleic acid is mRNA, tRNA (transfer RNA), rRNA (ribosomal RNA), or noncoding RNA (such as lncRNA (long noncoding RNA)). In embodiments, the template nucleic acids are on different regions of the same RNA nucleic acid sequence. In embodiments, the template nucleic acids are cDNA target nucleic acid sequences and before step i), the RNA nucleic acid sequences are reverse transcribed to generate the cDNA target nucleic acid sequences. In embodiments, the template nucleic acids are not reverse transcribed to cDNA. When mRNA is reverse transcribed an oligo(dT) primer can be added to better hybridize to the poly A tail of the mRNA. The oligo(dT) primer may include between about 12 and about 25 dT residues. The oligo(dT) primer may be an oligo(dT) primer of between about 18 to about 25 nt in length.

In embodiments, the polynucleotide includes a gene or a gene fragment. In embodiments, the gene or gene fragment is a cancer-associated gene or fragment thereof, T cell receptor (TCRs) gene or fragment thereof, or a B cell receptor (BCRs) gene, or fragment thereof. In embodiments, the gene or gene fragment is a CDR3 gene or fragment thereof. In embodiments, the gene or gene fragment is a T cell receptor alpha variable (TRAV) gene or fragment thereof, T cell receptor alpha joining (TRAJ) gene or fragment thereof, T cell receptor alpha constant (TRAC) gene or fragment thereof, T cell receptor beta variable (TRBV) gene or fragment thereof, T cell receptor beta diversity (TRBD) gene or fragment thereof, T cell receptor beta joining (TRBJ) gene or fragment thereof, T cell receptor beta constant (TRBC) gene or fragment thereof, T cell receptor gamma variable (TRGV) gene or fragment thereof, T cell receptor gamma joining (TRGJ) gene or fragment thereof, T cell receptor gamma constant (TRGC) gene or fragment thereof, T cell receptor delta variable (TRDV) gene or fragment thereof, T cell receptor delta diversity (TRDD) gene or fragment thereof, T cell receptor delta joining (TRDJ) gene or fragment thereof, or T cell receptor delta constant (TRDC) gene or fragment thereof. In embodiments, the polynucleotide includes genomic DNA, complementary DNA (cDNA), cell-free DNA (cfDNA), messenger RNA (mRNA), transfer RNA (tRNA), ribosomal RNA (rRNA), cell-free RNA (cfRNA), or noncoding RNA (ncRNA). In embodiments, the polynucleotide includes messenger RNA (mRNA), transfer RNA (tRNA), micro RNA (miRNA), small interfering RNA (siRNA), small nucleolar RNA (snoRNA), small nuclear RNA (snRNA), Piwi-interacting RNA (piRNA), enhancer RNA (eRNA), or ribosomal RNA (rRNA).

In embodiments, the methods and compositions described herein are utilized to analyze the various sequences of T cell receptors (TCRs) and B cell receptors (BCRs) from immune cells, for example various clonotypes. In embodiments, the target nucleic acid includes a nucleic acid sequence encoding a TCR alpha (TCRA) chain, a TCR beta (TCRB) chain, a TCR delta (TCRD) chain, a TCR gamma (TCRG) chain, or any fragment thereof (e.g., variable regions including VDJ or VJ regions, constant regions, transmembrane regions, fragments thereof, combinations thereof, and combinations of fragments thereof). In embodiments, the target nucleic acid includes a nucleic acid sequence encoding a B cell receptor heavy chain, B cell receptor light chain, or any fragment thereof (e.g., variable regions including VDJ or VJ regions, constant regions, transmembrane regions, fragments thereof, combinations thereof, and combinations of fragments thereof). In embodiments, the target nucleic acid includes a CDR3 nucleic acid sequence. In embodiments, the target nucleic acid includes a TCRA gene sequence or a TCRB gene sequence. In embodiments, the target nucleic acid includes a TCRA gene sequence and a TCRB gene sequence. In embodiments, the target nucleic acid includes sequences of various T cell receptor alpha variable genes (TRAV genes), T cell receptor alpha joining genes (TRAJ genes), T cell receptor alpha constant genes (TRAC genes), T cell receptor beta variable genes (TRBV genes), T cell receptor beta diversity genes (TRBD genes), T cell receptor beta joining genes (TRBJ genes), T cell receptor beta constant genes (TRBC genes), T cell receptor gamma variable genes (TRGV genes), T cell receptor gamma joining genes (TRGJ genes), T cell receptor gamma constant genes (TRGC genes), T cell receptor delta variable genes (TRDV genes), T cell receptor delta diversity genes (TRDD genes), T cell receptor delta joining genes (TRDJ genes), or T cell receptor delta constant genes (TRDC genes).

### EXAMPLES

### Example 1. Sequencing Beyond Typical Short Read Fragments

Typical short-read sequencing methods require de novo assembly of relatively short lengths of DNA (e.g., 35 to 150 base pairs), which makes resolving complex regions with mutations or repetitive sequences difficult. The application of those technologies to genome assemblies is limited by short sequence read length, which, by previous methods, is insufficient to resolve complex genome structure and to produce consistent genome assembly. In practice, researchers typically combine the output of long read sequencers (e.g., read length 10 kb, error rate 10-15%) with short read sequencing (error rate <1.5%). Further, short sequence reads have difficulties in obtaining phasing data (i.e., which variants are on the same chromosome) or detecting structural variants reliably. For example, inheritance patterns of genetic variation in complex traits may be influenced by interactions among multiple genes and alleles across long genomic distances (e.g., distances of greater than 2 kb). Examination of phased variants are critical for a greater understanding of the genetic basis of complex phenotypes (see, for example, Snyder, M. W., Adey, A., Kitzman, J. O. & Shendure, J. Nat. Rev. Genet. 16, 344-358 (2015)). Additionally, resolving long-range information at the molecular level within complex samples, e.g., cancer samples, is essential to assemble and phase variants of subpopulations of cells, as genetic drivers and important diagnostic biomarkers in cancers and other diseases (see, for example, Moncunill, V. et al. Nat. Biotechnol. 32, 1106-1112 (2014)). Described herein are methods for achieving greater read lengths by performing sequential sequencing of long polynucleotide strands using targeted primers (e.g., a plurality of sequencing primers that bind to different regions of a target polynucleotide). The long polynucleotides may be genomic (gDNA) with known regions or synthetic complements formed using interposing probes (IPPs) and the protocols described herein. The methods described herein are advantageous for sequencing two, three, or more target regions whose combined lengths plus the length of any intermediate sequence exceeds standard short-read lengths.

Described herein are methods that enable sequencing of a plurality of regions on the same polynucleotide. Significantly, the methods described herein take advantage of known sequences interspersed throughout the polynucleotide (e.g., either endogenous genomic regions and/or synthetically constructed polynucleotides. For example, two or more sequencing primers are complementary to endogenous sequences of the target polynucleotide and may be sequentially sequenced. Alternatively, the sequences in the target polynucleotide may be introduced into the target nucleic acid through integration of one or more probes including primer binding sites. For example, a first region of the target polynucleotide is sequenced by hybridizing and extending a first sequencing primer, for example, with labeled nucleotide analogues. Once sequencing of a first region is completed, for example, the sequenced strand may be removed (e.g., by digesting the strand or washing away with denaturing conditions). Alternatively, including a blocking element, such as terminating nucleotides, that is introduced following the sequencing of one region terminates extension on that specific strand. Subsequently, the next sequencing primer (e.g., a second, third, or fourth sequencing primer) is hybridized to another known region of the target nucleic acid (e.g., an endogenous sequence of the target nucleic acid, or an integrated probe sequence) and sequencing is re-initiated. Alternatively, the sequencing primers can be introduced in a single step and allowed to anneal to their respective region of interest. If all of the primers are loaded in a single step, the 3′ ends of the primers not being sequenced in the first series of sequencing cycles are non-extendable. Following sequencing and termination of the first sequenced strand, the next subsequent primer may be activated to initiate sequencing. Activating the primer may include removal of a blocking group on the primer (e.g., a blocking group with orthogonal removal conditions relative to the reversible terminators used during sequencing). This stepwise process of sequencing and terminating may also be utilized to facilitate long-range sequencing methods, for example, synthetic long reads, without the needs to remove intermediate sequencing products. These methods also enable sequencing of non-adjacent regions on a single polynucleotide strand. These novel methods reduce fluidic cycling requirements, reagent usage, and cycle times when sequencing multiple regions on one or more template polynucleotides.

Previous attempts to arrive at long-range sequencing data have been described elsewhere, for example U.S. Pat. No. 11,155,858, which is incorporated herein by reference. The interposing oligonucleotide probes as described herein contain binding sequences for the sequencing primers within the structure of the interposing oligonucleotide probes, and therefore, precludes the need to generate fragments of the integrated strands and ligate adapters containing sequences for sequencing primers. The exclusion of the fragmentation and adapter ligation steps reduces time to prepare sequencing libraries and reagent consumption. Furthermore, the method described herein includes contacting the template polynucleotides with blocking elements during sequencing to terminate extension at a specific region on an integrated strand, and the removal of the blocking element permits sequencing to re-initiate. This stepwise process of sequencing and terminating, as afforded from using sequencing primers and blocking elements, enable efficient generation of long-range sequencing reads from long template nucleic acids (e.g., 1 kb-50 kb) using read lengths of approximately 150-200 bp and can capture sequences of non-adjacent regions of template nucleic acids.

Embodiments herein provide certain advantages over other long-read sequencing methods, such as those that require ligation of amplification adapters followed by amplification and fragmentation (e.g., Nextera™ fragmentation). Such methods require multiple amplification steps, for example, to introduce sequencing adapters and barcodes prior to initiating sequencing. These steps, in contrast to the instant methods, may introduce biases (e.g., GC bias) or adversely affect the identification of rare variants through, for example, PCR errors. Methods that rely exclusively on reconstructing short reads based on barcode overlap are dependent on the barcodes being present, sequenced accurately, and in their entirety, to ensure accurate consensus sequence assembly. Including several size selection steps throughout library preparation may lead to reduced sequencing coverage of genomic areas where fragments are removed.

### Example 2. Sequencing with Targeted Sequencing Primers

Described herein are methods that enable sequencing of two, three, or more regions on a polynucleotide (e.g., the same single-stranded polynucleotide). Once sequencing of a first region is completed, for example, the sequenced strand may be removed (e.g., by digesting the strand or washing away with denaturing conditions). Alternatively, including a blocking element, such as modified nucleotides (e.g., terminating nucleotides), that is introduced following the sequencing of one region terminates extension on that specific strand. Subsequently, the next sequencing primer (e.g., a second, third, or fourth sequencing primer) is hybridized to another known region of the target nucleic acid (e.g., an endogenous sequence of the target nucleic acid, or an integrated probe sequence) and sequencing is re-initiated. Alternatively, the sequencing primers can be introduced in a single step and allowed to anneal to their respective region of interest. If all of the primers are loaded in a single step, the 3′ ends of the primers not being sequenced in the first series of sequencing cycles are non-extendable. Following sequencing and termination of the first sequenced strand, the next subsequent primer may be activated to initiate sequencing. Activating the primer may include removal of a blocking group on the primer (e.g., a blocking group with orthogonal removal conditions relative to the reversible terminators used during sequencing). The stepwise process of sequencing and terminating may also be utilized to facilitate long-range sequencing methods, for example, synthetic long reads, without the needs to remove intermediate sequencing products. These novel methods will, among other advantages, increase sequencing efficiency when targeting multiple regions on one or more template polynucleotides. In particular, these methods will provide for increase reagent savings, reduced labor costs, and more rapid sequencing turnaround times.

Patterned arrays are an important tool in biomedical research, providing a two-dimensional platform that arranges biological samples and enables high-throughput analyses. Delivering breakthroughs in proteomics, multiplexed immunoassays, and complex genomic analyses, microarrays can be designed to host thousands, or even ten-thousands, of features that can be subjected to simultaneous reaction conditions. In general terms, a target of interest (e.g., a protein or gene sequence) is immobilized as discrete features, or spots, on a substrate. Each feature may contain one to thousands of identical targets if subjected to an amplification technique. A successful detection event occurs when a labeled probe is brought into contact with the array, and if the probe interacts with the target, an increase of fluorescence intensity over a background level is produced, which can be measured using an appropriate detector.

Array techniques that rely on the random distribution of features typically suffer from a low ratio of incorporation event/pixel, due to a high number of dark pixels with no features (for example, if the density of features is too diffuse), or a high number of pixels that carry multiple overlapping features of different sequence (if the density of features is too concentrated) or both (due to the random nature of feature placement). An ideal and more efficient use of the imaging pixels occurs when the features on the surface are tightly packed, non-overlapping, and of similar size and intensity to each other.

The solid support herein may include particles of various compositions. For example, the solid support may include a plurality of wells, each well (i.e., a feature) including one or more particles that include polynucleotides. Polynucleotides on particles, confined to the restricted area of discrete, punctate clusters provide a more concentrated or intense signal, particularly when fluorescent probes are used in analytical operations, thereby providing higher signal-to-noise values and greater confidence in detection. In some embodiments, the particles may include solid core particles (e.g., a core made of glass, ceramic, metal, silica, magnetic material, or a paramagnetic material) with a plurality of particle polymers (e.g., a particle polymer moieties including polyacrylamide (Aam), poly-N-isopropylacrylamide, polyethylene glycol acrylate, methacrylate, acrylamide/N,N′-bis(acryloyl)cystamine (BACy), PEG/polypropylene oxide (PPO), polyacrylic acid, poly(hydroxyethyl methacrylate) (PHEMA), poly(methyl methacrylate) (PMMA), poly(N-isopropylacrylamide) (PNIPAAm), poly(lactic acid) (PLA), poly(lactic-co-glycolic acid) (PLGA), polycaprolactone (PCL), poly(vinylsulfonic acid) (PVSA), poly(L-aspartic acid), poly(L-glutamic acid), glicydyl methacrylate (GMA), hydroxyethylmethacrylate (HEMA), hydroxyethylacrylate (HEA), hydroxypropylmethacrylate (HPMA), polyethylene glycol methacrylate (PEGMA), polyethylene glycol acrylate (PEGA), isocyanatoethyl methacrylate (IEM), or a copolymer thereof) immobilized thereto. In embodiments, the solid core particle is a silica particle. In embodiments, the particle polymer includes PEGMA and GMA azide copolymers. In other embodiments, the solid support includes a polymer scaffold attached thereto.

Particles may be loaded into wells through several methods known in the art. For example, particles loading may simply be gravity driven. Gravity driven loading may also be accelerated by subsequently spinning down the array in a centrifuge, or with an orbital mixer to increase the particle settling rate. Such combinations are optimized so that no more than one particle is loaded into a given well, while achieve near complete coverage of the array with high uniformity. Additional particle loading techniques may involve agitating (e.g., vortexing), capillary assisted wetting, and/or centrifugation. In other embodiments, sonication and/or physical wiping with a flat tool may be used as a post-loading cleaning technique to reduce doubly-loaded wells and clear interstitial regions of particles. Post-cleaning may also simply consist of rinsing with a solvent, shaking, sonicating, wiping, or a combination thereof to remove non-specifically bound particles.

The particles are decorated with bioconjugate reactive moieties such that either before, or after, loading the particles into a well, one or more oligonucleotide moieties containing a complementary reactive bioconjugate reactive moiety may be bound to the particles (i.e., through the formation of a bioconjugate linker). In some embodiments, the oligonucleotides moiety is about 5 to about 45 nucleotides in length and is capable of hybridizing to a library nucleic acid molecule. In embodiments, the oligonucleotide moiety is capable of hybridizing to a complementary sequence of a template nucleic acid.

In order to obtain high-density particle loading with minimal background, the solid support of the array may be coated with a passivating polymer (e.g., a copolymer such as a silane functionalized polyethylene glycol (Si-PEG) copolymer or a silane functionalized poly(acrylamide) (Si-Pam)). The passivating polymer may be hydrophilic or hydrophobic (e.g., polyfluorinated polymer) and may be a comb polymer or brush polymer that is useful at preventing non-specific binding of additional agents to the array (e.g., oligonucleotides in solution). The array may include a photoresist (e.g., a fluorinated polymer later) prior to receiving an additional polymer coating (e.g., a poloxamer or alkoxysilyl polymer). The photoresist may be removed prior to the addition of the additional polymer using known techniques in the art (e.g., solvent removal). In embodiments, the additional polymer coating reduces the non-specific binding of oligonucleotide moieties. In embodiments, the particles are physiosorbed to the surface of the wells. Surprisingly, no covalent linkage between the particle and the array, nor hybridization of particles bearing an oligonucleotide sequence that is complementary to an immobilized primer on the array, is needed to retain the particles in the well. The interaction between the particle and the passivating polymer is sufficient to retain the particles in the well during amplification and sequencing.

The solid support may include a nanoimprint resist (e.g., organically-modified ceramic polymers, such as OrmoComp® from micro resist technology GmbH), which contains the plurality of wells. In embodiments, the nanoimprint resist includes a plurality of wells. Organically-modified ceramics are hybrid polymers with inorganic and organic moieties linked by stable covalent bonds and based on organically modified alkoxysilanes, functionalized organic polymers or both. See K. H. Haas, H. Wolter, “Synthesis, properties and applications of inorganic-organic copolymers (ORMOCER®s),” Current Opinions in Solid State and Materials Science, vol. 4, pp. 571-580, 1999, which is incorporated herein by reference. In nanolithography technologies, organically-modified ceramic polymers behave similarly to negative-tone photoresists, such as SU-8, and provide glass-like material properties after UV curing. Typical organically-modified ceramic polymers include oxides (e.g., SiO₂, ZrO, MgO, Al₂O₃, TiO₂ or Ta₂O₅), silicon oxide (—Si—O—) groups, polymerizable monomers (e.g., acryl or methacrylate monomers), and one or more alkyl moieties. In embodiments, the organically-modified ceramic polymer includes alkoxysilane and/or polymerized units of alkoxysilyl monomers. In embodiments, the organically-modified ceramic polymer is stable (i.e., does not measurably degrade) up to about 300° C. In embodiments, the solid support does not include particles.

Functionalization of solid support: A solid support (e.g., a flow cell) is prepared (e.g., washed in deionized water for 30 min, NaOH for 15 min, washed in deionized water for 30 min, HCl for 15 min, washed in deionized water for 30 min). The solid support is then coated in an in-house developed polymer which provides attachment points (e.g., reactive moieties capable of interacting with modified primers). The primers (e.g., forward and reverse primers) are typically 5′-phosphorothioate oligonucleotides incorporating any specific modifications required for grafting. The modified primer is incubated with the solid support for an hour and immobilized to the polymer.

Alternatively, a particle-loaded solid support is prepared. The following are examples of general particle loading techniques which may be applied to prepare the flow cell. To a patterned glass slide containing a photoresist with wells spaced 1.0 m apart (center-to-center), a copolymer of PEGMA-co-TMSPM is applied to the surface. The ratio of alkoxysilyl groups to PEG groups was 1:8. Approximately 4 mL of a colloidal solution containing approximately 10¹⁰ silica core particles (400 nm diameter) having a PEGMA-co-GMA azide shell were incubated with the array and allowed to settle into the wells. The ratio of azide functional groups to PEG functional groups was 1:4. The colloidal solution included ethanol, isopropyl alcohol, and water. The incubated array may be subjected to centrifugation (e.g., 2000 RPM for 12 minutes) or vortexed (e.g., 300 RPM for 12 minutes) to accelerate particle loading. Following loading, the array was washed 3× with water or ethanol and sonicated for 3 minutes. An additional wash step is performed with an ethanol, isopropyl alcohol, and water solution. The particle loaded array is allowed to dry or may be wiped down to accelerate surface drying. The array may be stored in an aqueous solution until ready to use.

To a patterned glass slide containing a photoresist with wells spaced 1.4 m apart (center-to-center), a copolymer of PEGMA-co-TMSPM is applied to the surface. The ratio of alkoxysilyl groups to PEG groups was 1:8. Approximately 4 mL of a colloidal solution containing approximately 10¹⁰ silica core particles (500 nm diameter) having a PEGMA-co-GMA azide shell were incubated with the array and allowed to settle into the wells. The ratio of azide functional groups to PEG functional groups was 1:4. The colloidal solution included ethanol, isopropyl alcohol, and water. The incubated array was subjected to centrifugation (e.g., 2000 RPM for 12 minutes) to accelerate particle loading. Following loading, the array was washed 3× with water or ethanol and sonicated for 3 minutes and allowed to dry. An additional wash step is performed with an ethanol, isopropyl alcohol, and water solution. The particle loaded array is allowed to dry or may be wiped down to accelerate surface drying. The array may be stored in an aqueous solution until ready to use.

Polymerized silica particles are added to a mixture of ethanol, isopropyl alcohol and water and sonicated. Into this solution was added a polymerized surface slide (i.e., a passivated slide). The slide and particle solution are shaken and incubated for 1 to 8 hours. Following this incubation period, the slides are shaken in an ethanol solution several times. To check the quantity of synthesized particles deposited onto the polymerized surface slides, phase contrast microscopy was performed on the slides. Polymerized silica particles with oligonucleotide primers in loading solution (TE buffer with NaCl which may optionally contain ethanol) is sonicated. Following sonication, the particles in solution was added to a tray containing patterned glass slide(s) that contain a resist, for example SU-8 and/or Ormocomp®, and shaken for 10 min. Following shaking, the tray is placed at 4° C. The slides are dried, washed and dried again. The slides were examined under microscope to check the quantity of polymerized silica particles with oligonucleotide primers particles deposited on the slides.

Library preparation (optional): Prior to sequencing, the input nucleic acid material may be fragmented using techniques known to those in the art. Three approaches available to fragment nucleic acid chains include: physical, enzymatic, and chemical. Nucleic acid fragmentation is typically done by physical methods (i.e., acoustic shearing and sonication) or enzymatic methods (i.e., non-specific endonuclease cocktails and transposase tagmentation reactions). Following fragmentation, the nucleic acid fragments are end-repaired or end-polished. Generally, a single adenine base is added to form an overhang via an A-tailing reaction. This “A” overhang allows adapters containing a single thymine overhanging base to base pair with the DNA fragments. Additional sequences such as adapters or primers may then be added using conventional means to permit platform specific sequences or to provide a binding site for sequencing primers.

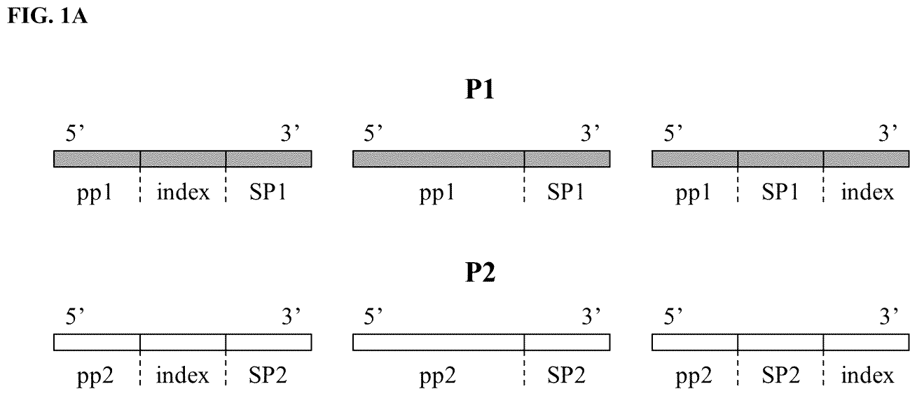

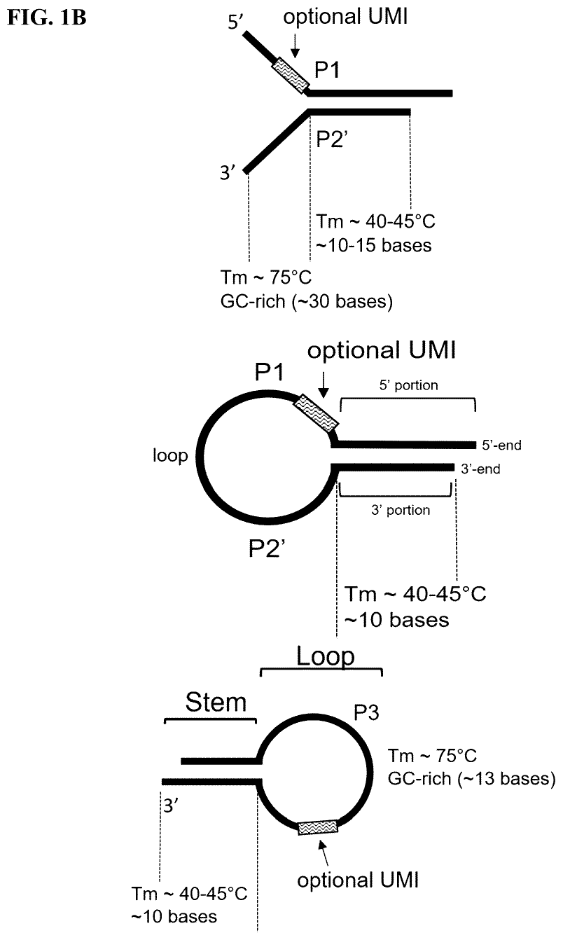

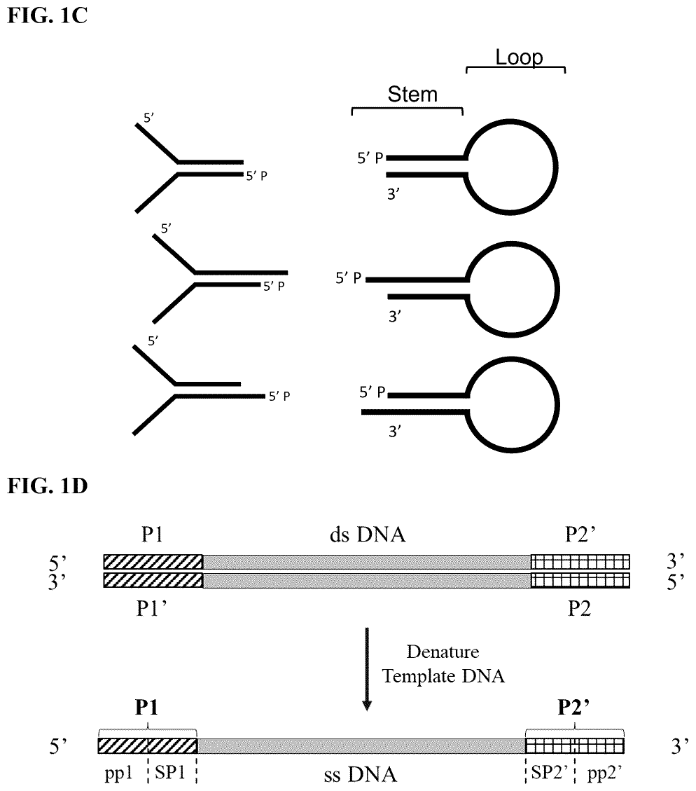

Adapters (optional): In some embodiments of the methods described herein, an adapter-target-adapter nucleic acid template is provided where two adapters are ligated to each respective end of a polynucleotide duplex. A polynucleotide duplex refers to a double-stranded portion of a polynucleotide, for example a polynucleotide desired to be sequenced. As shown in FIG. 1A, adapter sequences may be provided including various orientations of platform primer, index, and sequencing primer sequences. As depicted in FIG. 1B, in embodiments, a first adapter is a Y adapter (alternatively, this may be referred to as a forked adapter) that is ligated to one end of a polynucleotide duplex. The adapter is formed by annealing two single-stranded oligonucleotides, herein referred to as P1 and P2′. In some embodiments, the Y adapter includes (i) a first strand having a 5′-arm and a 3′-portion, and (ii) a second strand having a 5′-portion and a 3′-arm, wherein the 3′-portion of the first strand is substantially complementary to the 5′-portion of the second strand, and the 5′-arm of the first strand is not substantially complementary to the 3′-arm of the second strand. P1 and P2′ may be prepared by a suitable automated oligonucleotide synthesis technique. The oligonucleotides are partially complementary such that a 3′ end and/or a 3′ portion of P1 is complementary to the 5′ end and/or a 5′ portion of P2′. A 5′ end and/or a 5′ portion of P1 and a 3′ end and/or a 3′ portion of P2′ are not complementary to each other, in certain embodiments. When the two strands are annealed, the resulting Y adapter is double-stranded at one end (the double-stranded region) and single-stranded at the other end (the unmatched region), and resembles a ‘Y’ shape.

In embodiments, the adapter is a hairpin adapter (alternatively, it may be referred to as a stem-loop adapter, barbell, or hairpin loop adapter), as shown in FIG. 1B. In embodiments, the hairpin adapter includes a P1 priming site and a P2 priming site, and optionally a UMI, as depicted in the middle panel of FIG. 1B. The hairpin adapter may contain a P3 priming site and an optional UMI, as depicted in the bottom panel of FIG. 1B. In embodiments, the hairpin adapter includes a nucleic acid having a 5′-end, a 5′-portion, a loop, a 3′-portion and a 3′-end. The hairpin adapter includes a double-stranded region which has a moderate Tm (e.g., 40-45° C.) so that it is stable during ligation, and includes at least 10 nucleotides. The hairpin adapter also includes a loop region which has a primer sequence and has an elevated Tm (e.g., 75° C.) relative to the double stranded region to enable stable binding of a complementary sequencing primer. The loop region or the stem region of the hairpin may further include a barcode or Unique Molecular Identifier (UMI) using degenerate sequences. The UMI consists of 3-5 degenerate nucleotides. As shown in FIG. 1C, the double-stranded region of the hairpin adapter may be blunt-ended (top), it may have a 5′ overhang (middle), or a 3′ overhang (bottom). The overhang may include a single nucleotide or more than one nucleotide. The 5′ end of the double-stranded part of the hairpin adapter is phosphorylated. The presence of the 5′ phosphate group allows the adapter to ligate to the polynucleotide duplex.

In embodiments, a Y adapter is formed from cleavage in the loop of a hairpin adapter. For example, in embodiments disclosed herein relating a Y adapter, ligation may instead be to a hairpin adapter, followed by cleavage within the loop of the hairpin adapter to release two unpaired ends. In embodiments, a hairpin adapter includes a cleavable moiety (e.g., uracil) in the loop, and cleavage in the loop may be accomplished by the combined activities of uracil DNA glycosylase (UDG) and the DNA glycosylase-lyase endonuclease VIII. Suitable cleavage means include, for example, restriction enzyme digestion, in which case the cleavable site is an appropriate restriction site for the enzyme which directs cleavage of one or both strands of a duplex template; RNase digestion or chemical cleavage of a bond between a deoxyribonucleotide and a ribonucleotide, in which case the cleavable site may include one or more ribonucleotides; or chemical reduction of a disulfide linkage with a reducing agent (e.g., THPP or TCEP), in which case the cleavable site should include an appropriate disulfide linkage. In embodiments, the cleavable moiety is a diol linkage. It will be appreciated that more than one diol can be included at the cleavable site. One or more diol units may be incorporated into a polynucleotide using standard methods for automated chemical DNA synthesis. The diol linker is cleaved by treatment with any substance which promotes cleavage of the diol (e.g., a diol-cleaving agent). In embodiments, the diol-cleaving agent is periodate, e.g., aqueous sodium periodate (NaIO₄). Following treatment with the diol-cleaving agent (e.g., periodate) to cleave the diol, the cleaved product may be treated with a “capping agent” in order to neutralize reactive species generated in the cleavage reaction. Suitable capping agents for this purpose include amines, e.g., ethanolamine or propanolamine. In embodiments, the cleavable moiety is a modified nucleotide (e.g., uracil, 8oxoG, 5-mC, or 5-hmC) that is removed or nicked via a corresponding DNA glycosylase, endonuclease, or combination thereof. In embodiments, the adapter is a bubble adapter, including a complementary DNA region on each end, and a non-complementary loop region in the middle.

The single-stranded portions (the unmatched regions) of both P1 and P2′ have an elevated melting temperature (Tm) (e.g., about 75° C.) relative to their respective complements to enable efficient binding of surface primers and stable binding of sequencing primers. To achieve an elevated Tm in a reasonable length primer, the GC content is often >50% (e.g., approximately 60-75% GC content). In contrast to the single-stranded portions, a double-stranded region, in certain embodiments, has a moderate Tm (e.g., 40-45° C.) so that it is stable during ligation. In embodiments, a double-stranded region has an elevated Tm (e.g., 60-70° C.). In embodiments, the GC content of the double-stranded region is >50% (e.g., approximately 60-75% GC content). An unmatched region of P1 and P2′, in certain embodiments, are about 25-35 nucleotides (e.g., 30 nucleotides), whereas the double-stranded region is shorter, ranging about 10-20 nucleotides (e.g., 13 nucleotides) in total. In some embodiments, an internal region of P2′ may include an indexing tag. In embodiments, the indexing tag ranges from about 2 to 12 nucleotides (e.g., 8 nucleotides) in total. In embodiments, the Y adapters contain a region for hybridizing a sequencing primer, herein referred to as S1 and/or S2.

As shown in FIG. 1C, double-stranded region of the Y adapter may be blunt-ended (top), it may have a 3′ overhang (middle), or a 5′ overhang (bottom). The overhang may include a single nucleotide or more than one nucleotide. The 5′ end of the double-stranded part of the Y adapter is phosphorylated, i.e., the 5′ end of P2′. The presence of the 5′ phosphate group (referred to as 5′P in FIG. 1C) allows the adapter to ligate to the polynucleotide duplex. The 5′ end of P1 may be biotinylated or have a functional group at the end, thus enabling it to be immobilized on a surface (e.g., a planar solid support).

Adapter ligation (optional): A ligation reaction between two Y adapters and the DNA fragments is then performed using a suitable ligase enzyme (e.g., T4 DNA ligase) which joins one Y adapter to each end of the DNA fragment to form adapter-target-adapter constructs. Alternatively, a ligation reaction between a Y adapter and a hairpin adapter and the DNA fragments is performed, forming a “bobby pin” structure. Alternatively, a ligation reaction between two hairpin adapters and the DNA fragments is performed, forming a “dumbbell” structure. The products of this reaction can be purified from leftover unligated adapters by a number of means, including size-inclusion chromatography, preferably by electrophoresis through an agarose gel slab followed by excision of a portion of the agarose that contains the DNA greater in size that the size of the adapter.

The order of ligation events is not relevant, however for the purposes of discussion the terms ‘first’ and ‘second’ are used in reference to the sequence in which the adapter is ligated to the polynucleotide duplex. It is understood that the ligation of the Y adapter or the hairpin adapter may occur first, such that the resulting adapter-target-adapter constructs contain non-identical adapters.

Note, during this step it is possible to form adapter dimers (i.e., two adapters ligate together with no intervening template nucleic acid). There are several ways to reduce adapter dimer formation in the adapter ligation NGS library preparation described herein, including i) a stringent purification step (e.g., SPRI) after 3′ adapter ligation to remove non-ligated 3′ adapter molecules, prior to the second ligation of the 5′ adapter; ii) the use of A-tailed DNA and T-overhang adapters; iii) or utilizing alkaline phosphatase treatment after 3′ adapter ligation, before any SPRI cleanup, to remove 5′ phosphate group from the 3′ adapter to render any carryover 3′ adapter to be ligation incompatible and inert in the 5′ adapter ligation step.

Template amplification: Following construct formation, the constructs are amplified. The contents of an amplification reaction are known by one skilled in the art and include appropriate substrates (such as dNTPs), enzymes (e.g., a DNA polymerase) and buffer components required for an amplification reaction. Generally, amplification reactions require at least two amplification primers, often denoted ‘forward’ and ‘reverse’ primers (primer oligonucleotides) that are capable of annealing specifically to a part of the polynucleotide sequence (e.g., P1 and P2′ regions in FIG. 1A) to be amplified under conditions encountered in the primer annealing step of each cycle of an amplification reaction. In embodiments the forward and reverse primers include a sequence of nucleotides capable of annealing to a part of a primer-binding sequence in the polynucleotide molecule to be amplified (or the complement thereof if the template is viewed as a single strand) during the annealing step.

In embodiments, the amplification primers may be universal for all samples, or one of the forward or reverse primers may carry the tag sequence that codes for the sample source. The amplification primers may hybridize across the tag region of the ligated adapter, in which case unique primers will be needed for each sample nucleic acid. The amplification reaction may be performed with more than two amplification primers. In order to prevent the amplification of ligated adapter-adapter dimers, the amplification primers can be modified to contain nucleotides that hybridize across the whole of the ligated adapter and into the ligated template (or the dNTP's attached to the 3′ end thereof). This first amplification primer can be modified and treated to help prevent exonuclease digestion of the strands, and thus it may be advantageous to have a first amplification primer that is universal and can amplify all samples rather than modifying and treating each of the tagged primers separately. The tagged primer can be introduced as a sample specific third primer in the amplification reaction but does not need to be specially modified and treated.

Since the Y-adapters can be different lengths, the length of adapter sequence added to the 3′ and 5′ ends of each strand may be different. The amplification primers may also be of different lengths to each other and may hybridize to different lengths of the adapter, and therefore the length added to the ends of each strand can be controlled. The length of the added sequences may be 20-100 bases or more depending on the desired experimental design. The forward and reverse primers may be of sufficient length to hybridize to the whole of the adapter sequence and at least one base of the target sequence.

Primers may additionally include non-nucleotide chemical modifications, for example one or more phosphorothioate(s) to increase exonuclease resistance, again provided such that modifications do not prevent primer function. Modifications may, for example, facilitate attachment of the primer to a solid support, for example a biotin moiety. Certain modifications may themselves improve the function of the molecule as a primer, or may provide some other useful functionality, such as providing a site for cleavage to enable the primer (or an extended polynucleotide strand derived therefrom) to be cleaved from a solid support.

Amplification may also be performed using a first plurality of oligonucleotides and a second plurality of oligonucleotides, wherein one or more of the first plurality include a first sequence capable of hybridizing to a first endogenous region of a target polynucleotide, and wherein one or more of the second plurality include a second sequence capable of hybridizing to the complement of a second region of the target polynucleotide (see, U.S. Patent Application 63/311,576, which is incorporated herein by reference in its entirety). This method, for example has the advantage of not requiring extensive library prep or adapter ligation compared to existing commercial kit offerings, and allows for amplification and immobilization of an endogenous polynucleotide prior to extended-range targeted sequencing.

Sample immobilization: The term ‘solid-phase amplification’ as used herein refers to any nucleic acid amplification reaction carried out on or in association with a solid support such that all or a portion of the amplified products are immobilized on the solid support as they are formed. In particular, the term encompasses solid-phase polymerase chain reaction (solid-phase PCR) and solid phase isothermal amplification which are reactions analogous to standard solution phase amplification, except that one or both of the forward and reverse amplification primers is/are immobilized on the solid support. Although the invention encompasses ‘solid-phase’ amplification methods in which only one amplification primer is immobilized (the other primer usually being present in free solution), it is preferred for the solid support to be provided with both the forward and the reverse primers immobilized.

Nucleic acid molecules are hybridized in Tris HCl buffer with NaCl to a solid support (e.g., a flow cell) that contains forward and reverse nucleic acid primers. The library of nucleic acid molecules (approximately 1 pM concentration) is incubated for 15-30 min at 45° C. Surface amplification is carried out via any amplification method of choice (e.g., thermal cycling via PCR or isothermal eRCA). Alternatively, amplicons generated with biotin-labeled primers can be immobilized onto a solid support followed by denaturation to release the complementary strand. The monoclonal clusters can proceed to any necessary post-processing steps such as blocking of free 3′ ends, removal of select amplicons, or hybridization of a sequencing primer. Optionally, the clusters are quantified by introducing a nucleic acid stain (e.g., SYBR® Gold stain available from Thermo Fisher, Catalog #S11494 or a FAM (6-fluorescein amidite) labeled oligonucleotide) in the presence of a buffer is allowed to incubate with the amplicons for 10 minutes. After a wash, the substrate containing the stained amplicons is imaged and subjected to post-processing analysis to determine cluster size and brightness. After these steps, clusters are ready for sequencing in a sequencing-by-synthesis system.

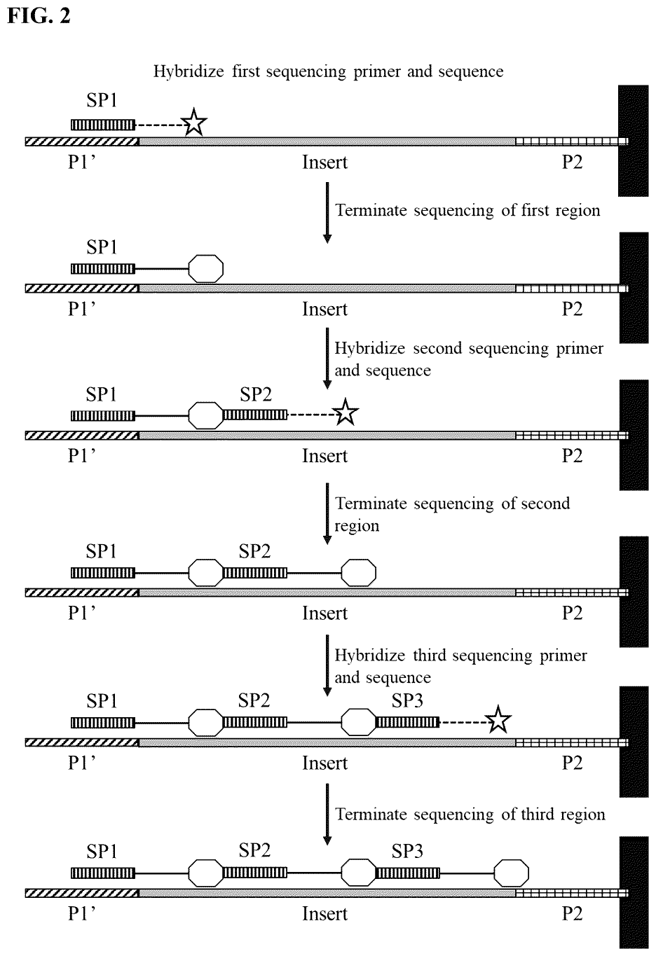

First region sequencing and termination: Sequencing is initiated by hybridizing a first sequencing primer to the polynucleotide template, and in the presence of a DNA polymerase and reversibly-terminated nucleotides, sequencing a first region of the polynucleotide. For example, to genomic DNA with known regions, two or more different primers are annealed to the known regions and sequenced in an iterative manner. FIG. 2 illustrates an immobilized template polynucleotide including primer binding sequences on each end (e.g., a P2 primer binding sequence on the 5′ end and a P1′ primer binding sequence on the 3′ end), wherein the template polynucleotide is immobilized to a solid support (illustrated as a dark rectangle). As described herein, in embodiments, adapters are optional, and the first sequencing primer may be designed such that it targets an endogenous sequence of the target polynucleotide. FIG. 2 further illustrates the steps of hybridizing the first sequencing primer (e.g., SP1) to an immobilized template polynucleotide including primer binding sequences on each end (e.g., a P2 primer binding sequence on the 5′ end and a P1′ primer binding sequence on the 3′ end), wherein the template polynucleotide is immobilized to a solid support (illustrated as a dark rectangle), and sequencing a first region by extending the primer with a polymerase to incorporate and detect labeled nucleotides (depicted as a dashed line and star). Following sequencing of the first region, extension is terminated (e.g., by incorporating a ddNTP (shown here as an octagon), or by removing the sequenced strand).

Termination includes methods or reagents that preclude further sequencing of the first region while sequencing any subsequent regions of the template polynucleotide. For example, termination occurs by contacting the first region with a dideoxy nucleotide triphosphate (e.g., ddATP, ddTTP, ddCTP, ddGTP) or a combination thereof. The addition of a dideoxy nucleotide triphosphate, which lacks a 3′-OH group required for the formation of a phosphodiester bond with an adjacent nucleotide, can inhibit further sequencing. In embodiments, termination includes the incorporated of unmodified dNTPs. In embodiments, a chain-terminating nucleotide includes any nucleotide or nucleotide analog that lacks a 3′-OH and is a substrate for a polymerase. For example, azidothymidine (AZT) is a chain-terminating nucleotide analog. Other chain-terminating nucleoside analogs are described, for example, in Yamamoto J et al. Molecules. 2016; 21(6):766, which is incorporated herein by reference in its entirety.

Alternatively, termination can include the introduction of reversibly-terminated nucleotides that are cleaved under different conditions than the modified nucleotides used when sequencing the second region. For example, terminating sequencing occurs by contacting the first region with reversibly-terminated nucleotides containing 3′-O-allyl group, which is cleaved by transition metal catalysis, a 3′-O-methoxymethyl group, which is cleaved by acid, a 3′-O-nitrobenzyl group, which is cleaved by light, or 3′-O—NH₂ group which is cleaved via buffered nitrous acid; and sequencing the second region uses RTs cleaved via a reducing agent (e.g., azidomethyl, or disulfide containing RTs). In some embodiments, terminating sequencing of the first region can include exhausting available template in the first sequencing read.

As an additional alternative, termination may include hybridizing an extension blocking primer (e.g., a blocker oligonucleotide) downstream of the sequenced strand, for example, such that additional labeled nucleotide may not be incorporated. Blocker oligonucleotides may include non-canonical nucleobase units and/or non-conventional linkages which result in the blocking oligonucleotide having high affinity for the targeted region (i.e., greater affinity for the targeted region than a correspondingly unmodified oligonucleotide). Non-canonical nucleobase units and/or non-conventional linkages suitable for incorporation into blocker oligonucleotides include, but are not limited to, LNAs and 2′-O-Me nucleobase units. Because of their high affinity for target nucleic acid, when contacted by the advancing portion of a polymerase (e.g., a DNA polymerase), blocking oligonucleotides halt strand extension by the polymerase. Typically, blocker oligonucleotides are non-extendible in the presence of a DNA polymerase, and thus are designed to prevent the initiation of DNA synthesis therefrom, e.g., by inclusion of a nucleobase unit at their 3′ end that prevents polymerase-based extension from the oligonucleotide. Additional extension blocking primers are described in U.S. Pat. Pub. US2014/0329282, which is incorporated herein by reference in its entirety.

Sequencing of additional target regions: Once sequencing of the first region is terminated, a second sequencing primer is then hybridized to a second known region of the polynucleotide. In some embodiments, the second sequenced region is located 5′ to the first sequenced region. In embodiments, the second sequenced region is adjacent to the first sequenced region. In embodiments, the second sequenced region is not adjacent to the first sequenced region. In some embodiments, following sequencing termination of the first region, the second primer is hybridized to the second region and is followed by sequencing of the second region. FIG. 2 further illustrates the steps of hybridizing a second sequencing primer (e.g., SP2) to a known region of the insert and sequencing the second region. Sequencing of the second region is terminated, and then a third sequencing primer (e.g., SP3) is hybridized to a third region of the insert and sequencing of the third region is performed. Once sequencing of the third region is complete, extension is terminated once again. Although only three regions of the template polynucleotide as shown as being targeted by sequencing primers and sequenced, it will be understood that additional primers may be designed to target additional regions for sequencing. The second sequenced region, or any sequenced region, may also contain an index sequence.

An alternate method for sequencing two or more regions on the same polynucleotide strand involves hybridizing a sequencing primer to the second target region (or third target region, fourth target region, etc.), extending in the presence of a polymerase and detectable nucleotides, and then switching to a sequencing reaction mixture with native nucleotides and performing a runoff extension (i.e., extending to a sufficient length). For example, runoff extension may be initiated once the index sequence in the second region has been completely acquired. Performing runoff extension is an alternative to terminating the extension, and reduces costs associated with performing sequencing cycles including modified nucleotides. Once extension of the second target region (or third target region, fourth target region, etc.) has been completed, a sequencing primer complementary to the first region is hybridized, and extension in the presence of a polymerase and detectable nucleotides is performed.

Sequencing can be carried out using any suitable sequencing-by-synthesis technique, wherein nucleotides are added successively to a free 3′ hydroxyl group, resulting in synthesis of a polynucleotide chain in the 5′ to 3′ direction. In embodiments, the identity of the nucleotide added is determined after each nucleotide addition. In some embodiments, terminating sequencing of the second region can include exhausting available template in the second sequencing read.

In embodiments, the sequencing method relies on the use of modified nucleotides that can act as reversible reaction terminators. Once the modified nucleotide has been incorporated into the growing polynucleotide chain complementary to the region of the template being sequenced there is no free 3′-OH group available to direct further sequence extension and therefore the polymerase cannot add further nucleotides. Once the identity of the base incorporated into the growing chain has been determined, the 3′ reversible terminator may be removed to allow addition of the next successive nucleotide. Such reactions can be done in a single experiment if each of the modified nucleotides has attached a different label, known to correspond to the particular base, to facilitate discrimination between the bases added at each incorporation step. Alternatively, a separate reaction may be carried out containing each of the modified nucleotides separately.

The modified nucleotides may carry a label (e.g., a fluorescent label) to facilitate their detection. Each nucleotide type may carry a different fluorescent label. However, the detectable label need not be a fluorescent label. Any label can be used which allows the detection of an incorporated nucleotide. One method for detecting fluorescently labeled nucleotides includes using laser light of a wavelength specific for the labeled nucleotides, or the use of other suitable sources of illumination. The fluorescence from the label on the nucleotide may be detected (e.g., by a CCD camera or other suitable detection means).

Use of the sequencing method outlined above is a non-limiting example, as essentially any sequencing methodology which relies on successive incorporation of nucleotides into a polynucleotide chain can be used. Suitable alternative techniques include, for example, pyrosequencing methods, FISSEQ (fluorescent in situ sequencing), MPSS (massively parallel signature sequencing), or sequencing by ligation-based methods.

### Example 3. Interposing Probes for Targeted Sequencing

Described herein are methods for achieving greater read lengths by integrating specialized interposing probes, also referred to herein as probe inserts, into the target polynucleotide, in combination with the sequential targeted sequencing methods described supra.

### Method: Interposing Probe Hybridization, Extension, and Ligation

Briefly, FIG. 3A is an overview of a non-limiting example of an interposing probe (IPP), and includes a loop region, a stem region, and two hybridization pads. The loop region may include about 10 to about 20 random nucleotides. In embodiments, the loop region includes between 5 to 15 N nucleotides (e.g., 5, 6, 7, 8, 9, 10, 11, 12, 13, 14, or 15 N nucleotides). Such random sequences may be referred to as molecular barcodes or unique molecular identifiers (UMI). In embodiments, the IPP includes a primer binding sequence. In embodiments, a primer binding sequence (e.g., a sequencing primer binding sequence) is located in the loop region. In embodiments of the methods described herein, synthetic long reads are constructed by aligning all sequencing reads that contain the same sequencing primer binding sequence. In embodiments, sequencing reads are additionally aligned based on the occurrence of the same UML. Each sequencing primer binding sequence (or target/primer binding sequence combination) is unique, although rare multiple occurrences can be treated bioinformatically. The length of the sequencing primer binding sequence and/or UMI determines the number of target sequences and can be optimized for a given sequencing application. Aside from forming the backbone for long read alignment, the introduction of UMIs into sequencing libraries prior to target amplification by PCR has been shown to dramatically increase the sensitivity for rare mutations and enable absolute read counting. The stem region includes two known sequences capable of hybridizing to each other, ranging from about 5 to about 10 nucleotides, and is stable (i.e., capable to remaining hybridized together) at approximately a maximum temperature of 37° C., and unhybridizes (i.e., denatures) at temperatures greater than 50° C. Finally, the hybridization pads each include about 3 to about 5 known nucleotides (e.g., AGTCG for pad 1, and GGGAG for pad 2) and are capable of hybridizing to single-stranded template nucleic acids (i.e., they are a complement to the original target). Hybridization pad sequences may be designed such that each IPP hybridizes to the target polynucleotide at regularly spaced intervals, or they may be designed such that the IPPs hybridize to specific, non-adjacent target regions. FIG. 3B depicts the interposing barcode when the stem regions are denatured. Additionally, flanking adapters are included which may target either the 3′ end of the template DNA (see, FIG. 3C) or the 5′ end of the template DNA (see, FIG. 3D).

To an isolated DNA, sample interposing probes (as described herein) are added at an appropriate concentration such that there are approximately 50-100 bases between each IPP (see, FIG. 4). In some embodiments, each IBC hybridized on a polynucleotide includes a unique priming sequence on the 5′ end. A non strand-displacing polymerase (e.g., Klentaq, T4, T7, Bst, Phusion, Tfl, Pfu, or Stoffel fragment) extends the complement strand to generate an extension segment, and a ligase ligates the ends of the extension segment together with the next interposing barcode to produce a single integrated strand. The non-strand displacing polymerase can either be a naturally occurring enzyme, or one that is specifically engineered to minimize strand displacement.

As even “non-strand displacing” DNA polymerases can have a slight ability to displace a DNA oligonucleotide from a template strand of DNA, the hybridization of the oligonucleotide can be enhanced in order to stop strand displacement by the polymerase. Prevention of displacement can be achieved by using modifications to the oligonucleotide itself or by using additives that either stabilize the hybridization of the oligonucleotide or that stop the polymerase. Modifications to the oligonucleotides that reduce or inhibit the strand displacement activity of the polymerase are for instance 2′ fluoro nucleosides, PNAs, ZNAs, G-Clamps (U.S. Pat. No. 6,335,439, a cytosine analogue capable of clamp binding to guanine) or LNAs (US 2003/0092905; U.S. Pat. No. 7,084,125).

Optionally, the template DNA sample is washed away, and the resultant integrated strand may be subjected to reaction conditions (e.g., elevated temperature or denaturing additives) such that the stem regions of interposing barcodes and/or any secondary structures present denature to form a linear integrated strand, as schematically shown in FIG. 4. The integrated strand may be amplified using methods known to those skilled in the art (e.g., standard PCR amplification or rolling circle amplification) and subjected to standard library preparation methods as known to those skilled in the art and described herein. The integrated strand may serve as the input DNA with any commercially available library preparation kit. A variety of kits for making sequencing libraries from DNA are available commercially. Libraries may be prepared as described supra.

### Method: Probe Insert Integration Through Cas/Argonaute Systems

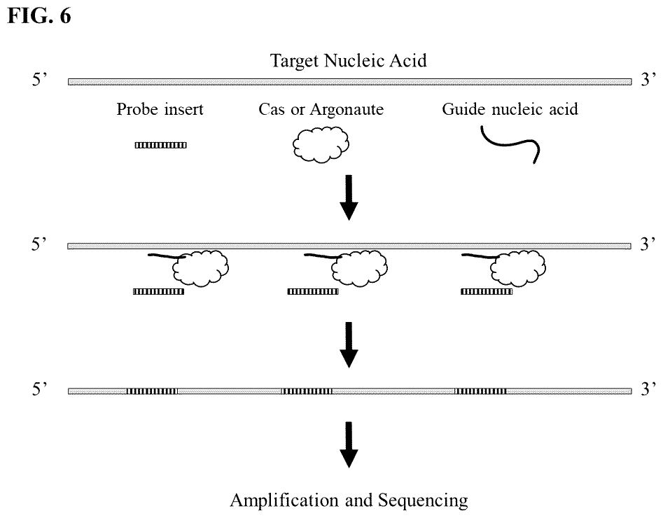

An alternate embodiment for inserting sequencing primer binding sequences into a target polynucleotide is described herein. Briefly, a protein integration complex (PIC) (e.g., a complex including a Cas or Argonaute protein, and a guide nucleic acid) is contacted with a target nucleic acid, in the presence of an oligonucleotide to be integrated into the target nucleic acid (e.g., a probe insert), as depicted in FIG. 6. The PIC integrates the probe insert into the target polynucleotide sequence, wherein the probe insert includes a sequencing primer binding site. In some embodiments, the PIC is a Type I CRISPR-Cas PIC or a variant thereof. In some embodiments, the PIC is a Type II CRISPR-Cas PIC or a variant thereof. In some embodiments, the PIC is a Type III CRISPR-Cas PIC or a variant thereof. In some embodiments, the Cas protein or the variant thereof is a Cas9 protein or a variant thereof. The use of CRISPR-Cas and related proteins in polynucleotide amplification are discussed in PCT Publ. Nos. WO2016/014409 and WO2016/077350, each of which are hereby incorporated by reference in their entireties. CRISPR/Cas9, for example, depends on a small RNA to introduce a site-specific double-stranded break. The endonuclease Cas9 only requires a 20-nucleotide ‘guide RNA’ (sgRNA) that base pairs with the target DNA and the presence of a DNA ‘protospacer-adjacent’ motif (PAM), a short DNA sequence adjacent to the complementary region that varies according to the bacterial species of the Cas9 protein being used, to match a DNA target sequence (see, e.g., Jacinto F V et al. J. Cell. Mol. Med. 2020; 24(7):3766-78, which is incorporated herein by reference in its entirety). In vitro editing using a Cas9-sgRNA complex to insert genes into a target nucleic acid sequence has been reported (see, e.g., Liu Y et al. mBio. 2015; 6(6):e01714-15, which is incorporated herein by reference in its entirety) and includes the use of a polymerase, such as T4 DNA polymerase to repair the Cas9-generated sticky ends in the target DNA.

Prokaryotic Argonaute proteins and their use in DNA-guided nicking and editing are discussed further in Lee K Z et al. Nucleic Acids Res. 2021; 49(17): 9926-37, Guo X et al. Front. Microbiol. 2021; 12: 654345, and Cao Y et al. Cell Discov. 2019; 5:38, each of which is incorporated herein by reference in its entirety. In some embodiments, the Argonaute protein is a Natronobacterium gregorgi Argonaute (NgAgo), or a variant thereof. Long prokaryotic Argonaute proteins (pAgos) are programmable endonucleases that have recently been proposed as flexible tools for genome editing (see, e.g., Hegge J W et al. Nat. Rev. Microbiol. 2018; 16(1):5-11, which is incorporated herein by reference). These enzymes bind single-stranded DNA and/or RNA molecules as guides, which then prime the enzyme for nicking of complementary target DNA, RNA, or both. Double stranded DNA cleavage requires two complementary guides, which may induce DNA repair and editing. Unlike Cas9-based gene editing strategies, however, pAgos have the distinct advantage of not requiring a protospacer adjacent motif (PAM) for function. Thus, pAgos are not limited to targets flanked by PAM sites and can potentially cut any DNA target regardless of composition.

The top panel of FIG. 6 illustrates the target DNA (e.g., a single-stranded polynucleotide), and the components of the PIC, including a probe insert, a Cas or Argonaute protein (e.g., a Cas protein (e.g., Cas9), a Argonaute (e.g., Natronobacterium gregorgi Argonaute, or NgAgo), or a variant thereof), and a guide nucleic acid (e.g., a guide RNA or guide 5′ phosphorylated single-stranded nucleic acid), wherein the guide nucleic acid includes a target-specific nucleotide region complementary or substantially complementary to a region of the target nucleic acid. The middle panel of FIG. 6 illustrates targeting of the probe insert by the Cas9 or NgAgo protein complex to the guide nucleic acid-specific target region. Target nucleic acid cleavage and probe insert integration then occur (not shown). The bottom panel of FIG. 6 illustrates the resulting template nucleic acid containing the integrated probe inserts, which may then be amplified and sequenced, for example, using a sequencing process as illustrated in FIGS. 5A-5B with sequencing primers complementary to the probe insert sequences.

### Sequencing of Primer-Integrated Polynucleotides

DNA libraries are prepared as described in Example 2, for example, through solid-phase amplification to generate polynucleotide strands attached to a support. Sequencing is initiated by hybridizing a first sequencing primer to a unique priming region on a first flanking adapter (see FIG. 5A), and in the presence of a DNA polymerase and reversibly-terminated nucleotides, sequencing a first target region. After the first region is sequenced, sequencing is terminated. Termination includes methods or reagents that preclude further sequencing of the first target region while sequencing the second region. For example, termination occurs by contacting the first region with a dideoxy nucleotide triphosphate (e.g., ddATP, ddTTP, ddCTP, ddGTP) or a combination thereof. The addition of a dideoxy nucleotide triphosphate which lacks a 3′-OH group required for the formation of a phosphodiester bond with an adjacent nucleotide, can inhibit further sequencing. In some embodiments, terminating sequencing of the first target region can include exhausting available template in the first sequencing read. Alternatively, the sequencing primers can be introduced in a single step and allowed to anneal to their respective region of interest. If all of the primers are loaded in a single step, the 3′ ends of the primers not being sequenced in the first series of sequencing cycles are non-extendable. Following sequencing and termination of the first sequenced strand, the next subsequent primer may be activated to initiate sequencing. Activating the primer may include removal of a blocking group on the primer (e.g., a blocking group with orthogonal removal conditions relative to the reversible terminators used during sequencing).

Alternatively, termination can include the introduction of reversibly-terminated nucleotides that are cleaved under different conditions than the modified nucleotides used when sequencing the second polynucleotide strand. For example, terminating sequencing occurs by contacting the first region with reversibly-terminated nucleotides containing 3′-O-allyl group, which is cleaved by transition metal catalysis, a 3′-O-methoxymethyl group, which is cleaved by acid, a 3′-O-nitrobenzyl group, which is cleaved by light, or 3′-O—NH2 group which is cleaved via buffered nitrous acid; and sequencing the second polynucleotide strand uses RTs cleaved via a reducing agent (e.g., azidomethyl, or disulfide containing RTs).

Once sequencing of the first region is terminated, the process of sequencing primer hybridization, sequencing, and sequencing termination is repeated for a second target region (and/or a third target region, a fourth target region, etc.). FIG. 5A shows hybridization of a second sequencing primer (e.g., SP2) to the first interposing probe and sequencing in the presence of a polymerase and detectable nucleotides a second region of the polynucleotide, followed by termination of extension through incorporation of a ddNTP. As shown in FIG. 5B, a third sequencing primer (e.g., SP3) is then hybridized to the second interposing probe, and a third region of the polynucleotide is sequenced in the presence of a polymerase and detectable nucleotides. Sequencing of the third region is then terminated through incorporation of a ddNTP. A fourth sequencing primer is then hybridized to the third interposing probe, and a fourth region (e.g., the 5′ end) of the polynucleotide is sequenced in the presence of a polymerase and detectable nucleotides. Sequencing of the fourth region is then terminated through incorporation of a ddNTP. While each sequencing primer and corresponding sequenced region of the template polynucleotide are illustrated as being spaced in regular intervals, it will be understood that each sequenced region may be of varying lengths, and sequencing primers may be targeted to non-adjacent portions of the template polynucleotide. In some embodiments, a UMI is sequenced in each target region, uniquely identifying each sequencing read. In embodiments, each sequencing read is uniquely identified based on the known sequence of the targeted sequencing primer used in sequencing. Sequencing can be carried out using any suitable sequencing-by-synthesis technique, wherein nucleotides are added successively to a free 3′ hydroxyl group, resulting in synthesis of a polynucleotide chain in the 5′ to 3′ direction. In embodiments, the identity of the nucleotide added is determined after each nucleotide addition. In some embodiments, terminating sequencing can include exhausting available template for a given sequencing read (e.g., the final sequencing read).

In embodiments, sequencing is performed according to a “sequencing-by-binding” method (see, e.g., U.S. Pat. Pubs. US2017/0022553 and US2019/0048404, each of which is incorporated herein by reference in its entirety), which refers to a sequencing technique wherein specific binding of a polymerase and cognate nucleotide to a primed template nucleic acid molecule (e.g., blocked primed template nucleic acid molecule) is used for identifying the next correct nucleotide to be incorporated into the primer strand of the primed template nucleic acid molecule. The specific binding interaction need not result in chemical incorporation of the nucleotide into the primer. In some embodiments, the specific binding interaction can precede chemical incorporation of the nucleotide into the primer strand or can precede chemical incorporation of an analogous, next correct nucleotide into the primer. Thus, detection of the next correct nucleotide can take place without incorporation of the next correct nucleotide.

Once data is available from the sequencing reaction(s), initial processing (often termed “pre-processing”) of the sequences is typically employed prior to annotation. Pre-processing includes filtering out low-quality sequences, sequence trimming to remove continuous low-quality nucleotides, merging paired-end sequences, or identifying and filtering out PCR repeats using known techniques in the art. The sequenced reads may then be assembled and aligned using bioinformatic algorithms known in the art. As illustrated in FIG. 5C, reads may be aligned (e.g., bioinformatically aligned) based on the presence of known features, for example, overlapping sequencing primer binding sequences or other landmarks present in each IPP.

What is claimed is:

1. A method of sequencing a single-strand polynucleotide, said method comprising:
(a) hybridizing a first sequencing primer to a first primer binding sequence of the single-strand polynucleotide and incorporating with a polymerase one or more nucleotides into the sequencing primer to create a first extension strand, and detecting the one or more incorporated nucleotides so as to identify each incorporated nucleotide in said first extension strand; and
(b) contacting the first extension strand with a first blocking element thereby terminating extension of the first extension strand thereby forming a first blocked extension strand;
(c) repeating steps (a) and (b), wherein each repetition of steps (a) and (b) comprises hybridizing a different sequencing primer to a different primer binding sequence of the single-strand polynucleotide; and
(d) hybridizing a terminal sequencing primer to a terminal primer binding sequence of the single-strand polynucleotide and incorporating with a polymerase one or more nucleotides into the terminal sequencing primer to create a terminal extension strand, and detecting the one or more incorporated nucleotides so as to identify each incorporated nucleotide in said extension strand.

2. The method of claim 1, wherein the single-stranded polynucleotide is 1 kb or greater.

3. The method of claim 1, wherein the single-stranded polynucleotide is about 1 kb to about 50 kb.

4. The method of claim 1, wherein the blocking element comprises a modified nucleotide triphosphate which lacks a 3′-OH.

5. The method of claim 1, wherein contacting the extension strand with a blocking element comprises hybridizing a blocking oligonucleotide downstream of the extension strand, wherein said blocking oligonucleotide comprises locked nucleic acids (LNAs), Bis-locked nucleic acids (bisLNAs), twisted intercalating nucleic acids (TINAs), bridged nucleic acids (BNAs), 2′-O-methyl RNA:DNA chimeric nucleic acids, minor groove binder (MGB) nucleic acids, morpholino nucleic acids, C5-modified pyrimidine nucleic acids, peptide nucleic acids (PNAs), phosphorothioate nucleic acids, or combinations thereof.

6. The method of claim 1, wherein steps (a) and (b) are repeated three or more times.

7. The method of claim 1, wherein each extension strand is independently about 5 to about 200 nucleotides in length.

8. The method of claim 1, wherein said single-stranded polynucleotide comprises three or more different sequencing primer binding sequences.

9. The method of claim 1, wherein said single-stranded polynucleotide is formed by
hybridizing two or more interposing oligonucleotide probes to a template nucleic acid molecule, wherein each of the interposing oligonucleotide probes comprises from 5′ to 3′: i. a first hybridization sequence complementary to a first sequence of said template nucleic acid; ii. a loop region comprising a sequencing primer binding sequence; and iii. a second hybridization sequence complementary to a second sequence of said template nucleic acid;
extending the 3′ end of each second hybridization sequence of said interposing oligonucleotide probes with one or more polymerases thereby forming an extension product of each of said oligonucleotide probes;
ligating the 3′ end of each of said extension products to the 5′ end of the adjacent extension products, thereby making an integrated strand comprising a complement of the template nucleic acid comprising a plurality of the oligonucleotide probes.

10. The method of claim 9, further comprising hybridizing a 5′ terminal oligonucleotide probe downstream of the one or more interposing oligonucleotide probes, wherein the 5′ terminal oligonucleotide probe comprises from 5′ to 3′: i. a hybridization sequence complementary to a third sequence of said template nucleic acid; and ii. a primer binding sequence; and hybridizing a 3′ terminal oligonucleotide probe upstream of the one or more interposing oligonucleotide probes, wherein the 3′ terminal oligonucleotide probe comprises from 3′ to 5′: i. a hybridization sequence complementary to a fourth sequence of said template nucleic acid; and ii. a primer binding sequence;
extending the 3′ end of the hybridization sequence of said 3′ terminal oligonucleotide probe with one or more polymerases thereby forming an extension product;
and ligating the 5′ end of the 5′ terminal oligonucleotide probe to the 3′ end of the adjacent extension product.

11. The method of claim 8, prior to step a) the method comprises amplifying the single-stranded polynucleotide.

12. The method of claim 11, wherein amplifying comprises bridge polymerase chain reaction (bPCR) amplification, solid-phase rolling circle amplification (RCA), solid-phase exponential rolling circle amplification (eRCA), solid-phase recombinase polymerase amplification (RPA), or solid-phase helicase dependent amplification (HIDA).

13. The method of claim 11, wherein amplifying comprises bridge polymerase chain reaction (bPCR) amplification.

14. The method of claim 11, wherein amplifying comprises solid-phase rolling circle amplification (RCA) or solid-phase exponential rolling circle amplification (eRCA).

15. The method of claim 9, wherein said first hybridization sequence, said second hybridization sequence, or both comprise one or more phosphorothioate-containing nucleotides or one or more LNAs.

16. The method of claim 1, wherein said single-stranded polynucleotide is attached to a solid support.

17. The method of claim 1, further comprising producing a plurality of sequencing reads; grouping sequencing reads based on co-occurrence of primer binding sequences; and within each group, computationally aligning the reads that belong to the same strand of an original sample polynucleotide based on the sequences of the primer binding sequences.

18. A method of sequencing a polynucleotide, said method comprising:
hybridizing a first sequencing primer to a first sequence of the polynucleotide and incorporating with a polymerase one or more nucleotides into the first sequencing primer to create a first extension strand, and detecting the one or more incorporated nucleotides so as to identify each incorporated nucleotide in said first extension strand; contacting the first extension strand with a blocking element thereby terminating extension of the first extension strand thereby forming a blocked first extension strand; followed by
hybridizing a second sequencing primer to a second sequence of the polynucleotide and incorporating with a polymerase one or more nucleotides into the second sequencing primer to create a second extension strand, and detecting the one or more incorporated nucleotides so as to identify each incorporated nucleotide in said second extension strand; contacting the second extension strand with a blocking element thereby terminating extension of the second extension strand thereby forming a blocked second extension strand; followed by
hybridizing a third sequencing primer to a third sequence of the polynucleotide and incorporating with a polymerase one or more nucleotides into the third sequencing primer to create a third extension strand, and detecting the one or more incorporated nucleotides so as to identify each incorporated nucleotide in said third extension strand.

19. A kit comprising:
(a) a plurality of interposing oligonucleotide probes capable of hybridizing to a template nucleic acid, said interposing oligonucleotide probes comprising from 5′ to 3′: i. a first hybridization sequence complementary to a first sequence of the template nucleic acid; ii. a loop region comprising a primer binding sequence; and iii. a second hybridization sequence complementary to a second sequence of the template nucleic acid;
(b) a plurality of 5′ terminal oligonucleotide probes capable of hybridizing to a template nucleic acid, said 5′ terminal oligonucleotide probes comprising from 5′ to 3′: i. a hybridization sequence complementary to a 5′ terminal sequence of the template nucleic acid, wherein the 5′ terminal sequence is upstream of the template nucleic acid sequence complementary to the interposing oligonucleotide probes; and ii. a primer binding sequence; and
(c) a plurality of 3′ terminal oligonucleotide probes capable of hybridizing to a template nucleic acid, said 3′ terminal oligonucleotide probes comprising from 3′ to 5′: i. a hybridization sequence complementary to a 3′ terminal sequence of the template nucleic acid, wherein the 3′ terminal sequence is downstream of the template nucleic acid sequence complementary to the interposing oligonucleotide probes; and ii. a primer binding sequence.

20. The kit of claim 19, wherein the plurality of 5′ terminal oligonucleotide probes comprise from 5′ to 3′: i. a first hybridization sequence complementary to a first 5′ terminal sequence of the template nucleic acid; ii. a loop region comprising a primer binding sequence; and iii. a second hybridization sequence complementary to a second 5′ terminal sequence of the template nucleic acid, wherein the first and second 5′ terminal sequences are 5′ of the template nucleic acid sequence complementary to the interposing oligonucleotide probes.
